SEQUENCE LISTING (181 sequences)

SEQ ID NO: 1 - protein, 49 aa, Artificial Sequence
  Synthetic Polypeptide
MIPGGLSEAKPATPEIQEIVDKVKPQLEEKTNETYGKLEAVQYKTQVLA

SEQ ID NO: 2 - protein, 24 aa, Artificial Sequence
  Synthetic Polypeptide
GTNYYIKVRAGDNKYMHLKVFKSL

SEQ ID NO: 3 - protein, 21 aa, Artificial Sequence
  Synthetic Polypeptide
EDLVLTGYQVDKNKDDELTGF

SEQ ID NO: 4 - protein, 9 aa, Artificial Sequence
  Synthetic Polypeptide
WTQPKNEHH

SEQ ID NO: 5 - protein, 9 aa, Artificial Sequence
  Synthetic Polypeptide
HLKHTDAQP

SEQ ID NO: 6 - protein, 9 aa, Artificial Sequence
  Synthetic Polypeptide
HDQDVLHAW

SEQ ID NO: 7 - protein, 9 aa, Artificial Sequence
  Synthetic Polypeptide
KFHRQEWAD

SEQ ID NO: 8 - protein, 9 aa, Artificial Sequence
  Synthetic Polypeptide
PEDFWDPEH

SEQ ID NO: 9 - protein, 9 aa, Artificial Sequence
  Synthetic Polypeptide
VVRTTGHVV

SEQ ID NO: 10 - protein, 9 aa, Artificial Sequence
  Synthetic Polypeptide
YWWFCTGQS

SEQ ID NO: 11 - protein, 9 aa, Artificial Sequence
  Synthetic Polypeptide
IHHRQARSL

SEQ ID NO: 12 - protein, 9 aa, Artificial Sequence
  Synthetic Polypeptide
SHRRRAYIW

SEQ ID NO: 13 - protein, 9 aa, Artificial Sequence
  Synthetic Polypeptide
WDSHHWRAP

SEQ ID NO: 14 - protein, 8 aa, Artificial Sequence
  Synthetic Polypeptide
DKRVKYGQ

SEQ ID NO: 15 - protein, 9 aa, Artificial Sequence
  Synthetic Polypeptide
SDWVYALQL

SEQ ID NO: 16 - protein, 6 aa, Artificial Sequence
  Synthetic Polypeptide
FWWFWY

SEQ ID NO: 17 - protein, 9 aa, Artificial Sequence
  Synthetic Polypeptide
VRDWPWNTF

SEQ ID NO: 18 - protein, 9 aa, Artificial Sequence
  Synthetic Polypeptide
QKKRDEDYI

SEQ ID NO: 19 - protein, 9 aa, Artificial Sequence
  Synthetic Polypeptide
GVHEEPRKL

SEQ ID NO: 20 - protein, 9 aa, Artificial Sequence
  Synthetic Polypeptide
EWWQKHWPS

SEQ ID NO: 21 - protein, 9 aa, Artificial Sequence
  Synthetic Polypeptide
NFFQRRWPG

SEQ ID NO: 22 - protein, 9 aa, Artificial Sequence
  Synthetic Polypeptide
DWWQAKWPH

SEQ ID NO: 23 - protein, 9 aa, Artificial Sequence
  Synthetic Polypeptide
GIWQSRWPG

SEQ ID NO: 24 - protein, 9 aa, Artificial Sequence
  Synthetic Polypeptide
GYWAAKWPG

SEQ ID NO: 25 - protein, 9 aa, Artificial Sequence
  Synthetic Polypeptide
GFYADHWPG

SEQ ID NO: 26 - protein, 9 aa, Artificial Sequence
  Synthetic Polypeptide
NWYQQRWPG

SEQ ID NO: 27 - protein, 9 aa, Artificial Sequence
  Synthetic Polypeptide
GFYARHWPG

SEQ ID NO: 28 - protein, 9 aa, Artificial Sequence
  Synthetic Polypeptide
DFWKAHWPG

SEQ ID NO: 29 - protein, 9 aa, Artificial Sequence
  Synthetic Polypeptide
DFYSVRWPG

SEQ ID NO: 30 - protein, 9 aa, Artificial Sequence
  Synthetic Polypeptide
YWAANHASK

SEQ ID NO: 31 - protein, 9 aa, Artificial Sequence
  Synthetic Polypeptide
IKRLEHWEY

SEQ ID NO: 32 - protein, 9 aa, Artificial Sequence
  Synthetic Polypeptide
EWDSPWSEN

SEQ ID NO: 33 - protein, 9 aa, Artificial Sequence
  Synthetic Polypeptide
KHKNLRWPF

SEQ ID NO: 34 - protein, 9 aa, Artificial Sequence
  Synthetic Polypeptide
RHFPKQTNW

SEQ ID NO: 35 - protein, 9 aa, Artificial Sequence
  Synthetic Polypeptide
VWGPEYQHQ

SEQ ID NO: 36 - protein, 9 aa, Artificial Sequence
  Synthetic Polypeptide
TWKNNGQDV

SEQ ID NO: 37 - protein, 9 aa, Artificial Sequence
  Synthetic Polypeptide
ATWLNYYLP

SEQ ID NO: 38 - protein, 9 aa, Artificial Sequence
  Synthetic Polypeptide
DQESLFLNN

SEQ ID NO: 39 - protein, 9 aa, Artificial Sequence
  Synthetic Polypeptide
GFYAQHWPD

SEQ ID NO: 40 - protein, 9 aa, Artificial Sequence
  Synthetic Polypeptide
GHYARYWPG

SEQ ID NO: 41 - protein, 9 aa, Artificial Sequence
  Synthetic Polypeptide
GFWASKWPG

SEQ ID NO: 42 - protein, 9 aa, Artificial Sequence
  Synthetic Polypeptide
GFWQRKWPN

SEQ ID NO: 43 - protein, 9 aa, Artificial Sequence
  Synthetic Polypeptide
VWPADNDLK

SEQ ID NO: 44 - protein, 9 aa, Artificial Sequence
  Synthetic Polypeptide
HWAWTSPGY

SEQ ID NO: 45 - protein, 9 aa, Artificial Sequence
  Synthetic Polypeptide
NFFQRRWPG

SEQ ID NO: 46 - protein, 9 aa, Artificial Sequence
  Synthetic Polypeptide
HHSHRLKGQ

SEQ ID NO: 47 - protein, 9 aa, Artificial Sequence
  Synthetic Polypeptide
YQNTIFLSI

SEQ ID NO: 48 - protein, 9 aa, Artificial Sequence
  Synthetic Polypeptide
FQDQFTWSQ

SEQ ID NO: 49 - protein, 9 aa, Artificial Sequence
  Synthetic Polypeptide
GEPHWPWQA

SEQ ID NO: 50 - protein, 9 aa, Artificial Sequence
  Synthetic Polypeptide
ADPRHPWVE

SEQ ID NO: 51 - protein, 9 aa, Artificial Sequence
  Synthetic Polypeptide
FHKRFQSQG

SEQ ID NO: 52 - protein, 9 aa, Artificial Sequence
  Synthetic Polypeptide
EWWQNRWPN

SEQ ID NO: 53 - protein, 9 aa, Artificial Sequence
  Synthetic Polypeptide
EWYQTRWPG

SEQ ID NO: 54 - protein, 9 aa, Artificial Sequence
  Synthetic Polypeptide
EFWQRHWPG

SEQ ID NO: 55 - protein, 9 aa, Artificial Sequence
  Synthetic Polypeptide
KFYERHWPG

SEQ ID NO: 56 - protein, 9 aa, Artificial Sequence
  Synthetic Polypeptide
GWWQRRWPG

SEQ ID NO: 57 - protein, 9 aa, Artificial Sequence
  Synthetic Polypeptide
RFKYFAHYQ

SEQ ID NO: 58 - protein, 9 aa, Artificial Sequence
  Synthetic Polypeptide
FHDFWHRRW

SEQ ID NO: 59 - protein, 9 aa, Artificial Sequence
  Synthetic Polypeptide
DWYHYWWEV

SEQ ID NO: 60 - protein, 9 aa, Artificial Sequence
  Synthetic Polypeptide
STRSIHVTT

SEQ ID NO: 61 - protein, 9 aa, Artificial Sequence
  Synthetic Polypeptide
KQHHHYLDK

SEQ ID NO: 62 - protein, 9 aa, Artificial Sequence
  Synthetic Polypeptide
HSAQDREIP

SEQ ID NO: 63 - protein, 9 aa, Artificial Sequence
  Synthetic Polypeptide
WVQSGYNSQ

SEQ ID NO: 64 - protein, 9 aa, Artificial Sequence
  Synthetic Polypeptide
AVFWGKWSD

SEQ ID NO: 65 - protein, 9 aa, Artificial Sequence
  Synthetic Polypeptide
QSFDKPWTT

SEQ ID NO: 66 - protein, 9 aa, Artificial Sequence
  Synthetic Polypeptide
HYPLKYSFE

SEQ ID NO: 67 - protein, 9 aa, Artificial Sequence
  Synthetic Polypeptide
WHHPWHRNR

SEQ ID NO: 68 - protein, 9 aa, Artificial Sequence
  Synthetic Polypeptide
DPWWAWVVW

SEQ ID NO: 69 - protein, 9 aa, Artificial Sequence
  Synthetic Polypeptide
FDNQDLIQY

SEQ ID NO: 70 - protein, 9 aa, Artificial Sequence
  Synthetic Polypeptide
EKKNWYKWD

SEQ ID NO: 71 - protein, 9 aa, Artificial Sequence
  Synthetic Polypeptide
DRHKSRWGI

SEQ ID NO: 72 - protein, 9 aa, Artificial Sequence
  Synthetic Polypeptide
LNPFTPSVT

SEQ ID NO: 73 - protein, 9 aa, Artificial Sequence
  Synthetic Polypeptide
YKGALLNHD

SEQ ID NO: 74 - protein, 9 aa, Artificial Sequence
  Synthetic Polypeptide
WKFRNTERG

SEQ ID NO: 75 - protein, 9 aa, Artificial Sequence
  Synthetic Polypeptide
YKVHQSSGG

SEQ ID NO: 76 - protein, 9 aa, Artificial Sequence
  Synthetic Polypeptide
FHPIAGRPW

SEQ ID NO: 77 - protein, 9 aa, Artificial Sequence
  Synthetic Polypeptide
FPNTSYDLQ

SEQ ID NO: 78 - protein, 9 aa, Artificial Sequence
  Synthetic Polypeptide
FAHYNLKSG

SEQ ID NO: 79 - protein, 9 aa, Artificial Sequence
  Synthetic Polypeptide
WHNYGESSG

SEQ ID NO: 80 - protein, 9 aa, Artificial Sequence
  Synthetic Polypeptide
KFYYADHQW

SEQ ID NO: 81 - protein, 9 aa, Artificial Sequence
  Synthetic Polypeptide
YTHADPHSQ

SEQ ID NO: 82 - protein, 9 aa, Artificial Sequence
  Synthetic Polypeptide
FGVPQLGAG

SEQ ID NO: 83 - protein, 9 aa, Artificial Sequence
  Synthetic Polypeptide
YSGFPFAGF

SEQ ID NO: 84 - protein, 9 aa, Artificial Sequence
  Synthetic Polypeptide
WFSWPYTPL

SEQ ID NO: 85 - protein, 9 aa, Artificial Sequence
  Synthetic Polypeptide
YYHPSIQST

SEQ ID NO: 86 - protein, 9 aa, Artificial Sequence
  Synthetic Polypeptide
FLGWKDTVV

SEQ ID NO: 87 - protein, 9 aa, Artificial Sequence
  Synthetic Polypeptide
DWWKWWWAK

SEQ ID NO: 88 - protein, 9 aa, Artificial Sequence
  Synthetic Polypeptide
NAGWPLVPE

SEQ ID NO: 89 - protein, 9 aa, Artificial Sequence
  Synthetic Polypeptide
YALDPFGGK

SEQ ID NO: 90 - protein, 9 aa, Artificial Sequence
  Synthetic Polypeptide
GYKFWGVSD

SEQ ID NO: 91 - protein, 9 aa, Artificial Sequence
  Synthetic Polypeptide
QGKQYILLR

SEQ ID NO: 92 - protein, 9 aa, Artificial Sequence
  Synthetic Polypeptide
YKRHSAHDY

SEQ ID NO: 93 - protein, 9 aa, Artificial Sequence
  Synthetic Polypeptide
WAQKSKVHQ

SEQ ID NO: 94 - protein, 9 aa, Artificial Sequence
  Synthetic Polypeptide
FTAVSKKDA

SEQ ID NO: 95 - protein, 9 aa, Artificial Sequence
  Synthetic Polypeptide
WGDKENIWF

SEQ ID NO: 96 - protein, 9 aa, Artificial Sequence
  Synthetic Polypeptide
WSGHPWVQK

SEQ ID NO: 97 - protein, 9 aa, Artificial Sequence
  Synthetic Polypeptide
YADYPLSPK

SEQ ID NO: 98 - protein, 9 aa, Artificial Sequence
  Synthetic Polypeptide
WKFRNTDRG

SEQ ID NO: 99 - protein, 9 aa, Artificial Sequence
  Synthetic Polypeptide
QTVATHYHY

SEQ ID NO: 100 - protein, 9 aa, Artificial Sequence
  Synthetic Polypeptide
WHAKHLLSH

SEQ ID NO: 101 - protein, 9 aa, Artificial Sequence
  Synthetic Polypeptide
SGIKKADSV

SEQ ID NO: 102 - protein, 9 aa, Artificial Sequence
  Synthetic Polypeptide
KANLINVKS

SEQ ID NO: 103 - protein, 9 aa, Artificial Sequence
  Synthetic Polypeptide
WKSHVEVRS

SEQ ID NO: 104 - protein, 9 aa, Artificial Sequence
  Synthetic Polypeptide
WVTQKYIIQ

SEQ ID NO: 105 - protein, 9 aa, Artificial Sequence
  Synthetic Polypeptide
WEHAKDWPT

SEQ ID NO: 106 - protein, 9 aa, Artificial Sequence
  Synthetic Polypeptide
FHSKVLDKA

SEQ ID NO: 107 - protein, 9 aa, Artificial Sequence
  Synthetic Polypeptide
YGAQKQAVW

SEQ ID NO: 108 - protein, 9 aa, Artificial Sequence
  Synthetic Polypeptide
FSASHFTSQ

SEQ ID NO: 109 - protein, 9 aa, Artificial Sequence
  Synthetic Polypeptide
  misc_feature (1)..(2): Xaa can be any naturally occurring amino acid
  misc_feature (4)..(4): Xaa can be any naturally occurring amino acid
  misc_feature (6)..(6): Xaa can be any naturally occurring amino acid
  misc_feature (8)..(8): Xaa can be any naturally occurring amino acid
XXAXKXDXQ

SEQ ID NO: 110 - protein, 112 aa, Artificial Sequence
  Synthetic Polypeptide
MIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 111 - protein, 112 aa, Artificial Sequence
  Synthetic Polypeptide
MIPRGLSEAKPATPEIQEIVDKVKPQLEEKTNETYGKLEAVQYKTQVLAGYWAAKWPGSTNYYIKVRAGDNKYMHLKVFNGPFPNTSYDLQADRVLTGYQVDKNKDDELTGF

SEQ ID NO: 112 - protein, 112 aa, Artificial Sequence
  Synthetic Polypeptide
MIPRGLSEAKPATPEIQEIVDKVKPQLEEKTNETYGKLEAVQYKTQVLANWYQQRWPGSTNYYIKVRAGDNKYMHLKVFNGPWHNYGESSGADRVLTGYQVDKNKDDELTGF

SEQ ID NO: 113 - protein, 112 aa, Artificial Sequence
  Synthetic Polypeptide
MIPRGLSEAKPATPEIQEIVDKVKPQLEEKTNETYGKLEAVQYKTQVLAVWGPEYQHQSTNYYIKVRAGDNKYMHLKVFNGPNAGWPLVPEADRVLTGYQVDKNKDDELTGF

SEQ ID NO: 114 - protein, 112 aa, Artificial Sequence
  Synthetic Polypeptide
MIPRGLSEAKPATPEIQEIVDKVKPQLEEKTNETYGKLEAVQYKTQVLAGHYARYWPGSTNYYIKVRAGDNKYMHLKVFNGPWAQKSKVHQADRVLTGYQVDKNKDDELTGF

SEQ ID NO: 115 - protein, 112 aa, Artificial Sequence
  Synthetic Polypeptide
MIPRGLSEAKPATPEIQEIVDKVKPQLEEKTNETYGKLEAVQYKTQVLAGFWASKWPGSTNYYIKVRAGDNKYMHLKVFNGPFTAVSKKDAADRVLTGYQVDKNKDDELTGF

SEQ ID NO: 116 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 117 - protein, 254 aa, Artificial Sequence
  Synthetic Polypeptide
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 118 - protein, 255 aa, Artificial Sequence
  Synthetic Polypeptide
MIPRGLSEAKPATPEIQEIVDKVKPQLEEKTNETYGKLEAVQYKTQVLAKEHGPDSWWSTNYYIKVRAGDNKYMHLKVFNGPQEKNQWVEEADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 119 - protein, 254 aa, Artificial Sequence
  Synthetic Polypeptide
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 120 - protein, 255 aa, Artificial Sequence
  Synthetic Polypeptide
MIPRGLSEAKPATPEIQEIVDKVKPQLEEKTNETYGKLEAVQYKTQVLAHAYGPRDWDSTNYYIKVRAGDNKYMHLKVFNGPPADHVLEEAADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 121 - protein, 254 aa, Artificial Sequence
  Synthetic Polypeptide
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 122 - protein, 255 aa, Artificial Sequence
  Synthetic Polypeptide
MIPRGLSEAKPATPEIQEIVDKVKPQLEEKTNETYGKLEAVQYKTQVLAKEYGPEEWWSTNYYIKVRAGDNKYMHLKVFNGPGDYEQVLIHADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 123 - protein, 398 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 124 - protein, 398 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 125 - protein, 398 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 126 - protein, 399 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFC

SEQ ID NO: 127 - protein, 398 aa, Artificial Sequence
  Synthetic Polypeptide
MIPRGLSEAKPATPEIQEIVDKVKPQLEEKTNETYGKLEAVQYKTQVLAFALPEFEYMSTNYYIKVRAGDNKYMHLKVFNGPPMIRRKNEVADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAFALPEFEYMSTNYYIKVRAGDNKYMHLKVFNGPPMIRRKNEVADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 128 - protein, 398 aa, Artificial Sequence
  Synthetic Polypeptide
MIPRGLSEAKPATPEIQEIVDKVKPQLEEKTNETYGKLEAVQYKTQVLAGGGGGGGGGSTNYYIKVRAGDNKYMHLKVFNGPGGGGGGGGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAGGGGGGGGGSTNYYIKVRAGDNKYMHLKVFNGPGGGGGGGGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 129 - protein, 255 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 130 - protein, 256 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFC

SEQ ID NO: 131 - protein, 255 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 132 - protein, 254 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFGGGGSGGGGSGGGGSGGGGSGGGGSGGGGSMIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 133 - protein, 164 aa, Artificial Sequence
  Synthetic Polypeptide
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEAISPPDAASAAPLRTITADTFRKLFRVYSNFLRGKLKLYTGEACRTGD

SEQ ID NO: 134 - protein, 337 aa, Artificial Sequence
  Synthetic Polypeptide
MPLLLLLPLLWAGALAIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAAGGGGSGGGGSGGGGSGGGGSAPTSSSAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEAISPPDAASAAPLRTITADTFRKLFRVYSNFLRGKLKLYTGEACRTGD

SEQ ID NO: 135 - protein, 337 aa, Artificial Sequence
  Synthetic Polypeptide
MPLLLLLPLLWAGALAIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAAGGGGSGGGGSGGGGSGGGGSAPTSSSAPPRLICDSRVLERYLLEAKEAEAITTGCAEHCSLNEAITVPDTKVNFYAWKRMEVGQQAVEVWQGLALLSEAVLRGQALLVASSQPWEPLQLHVDKAVSGLRSLTTLLRALGAQEAISPPDAASAAPLRTITADTFRKLFRVYSNFLRGKLKLYTGEACRTGD

SEQ ID NO: 136 - protein, 196 aa, Artificial Sequence
  Synthetic Polypeptide
MPLLLLLPLLWAGALAHGEGTFTSDVSSYLLEEQAAKEFIAWLVKGGGGSGGGGSGGGGSGGGGSIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAA

SEQ ID NO: 137 - protein, 235 aa, Artificial Sequence
  Synthetic Polypeptide
MPLLLLLPLLWAGALAHGEGTFTSDLSKQMEEEAVRLFIEWLKNGGPSSGAPPPSHGEGTFTSDVSSYLLEEQAAKEFIAWLVKGGGGSGGGGSGGGGSGGGGSIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGAA

SEQ ID NO: 138 - protein, 149 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGFAAAGGRAEQKLISEEDLGCAENLYFQGGAAGHHHHHH

SEQ ID NO: 139 - protein, 398 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 140 - protein, 398 aa, Artificial Sequence
  Synthetic Polypeptide
MIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGFAEAAAKEAAAKEAAAKEAAAKEAAAKEAAAKMIPRGLSEAKPATPEIQEIVDKVKPQLEEKTNETYGKLEAVQYKTQVLAREGRQDWVLSTNYYIKVRAGDNKYMHLKVFNGPWVPFPHQQLADRVLTGYQVDKNKDDELTGF

SEQ ID NO: 141 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPGGGGGGGGGADRVLTGYQVDKNKDDELTGF

SEQ ID NO: 142 - protein, 103 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPADRVLTGYQVDKNKDDELTGF

SEQ ID NO: 143 - protein, 112 aa, Artificial Sequence
  Synthetic Polypeptide
MIPRGLSEAKPATPEIQEIVDKVKPQLEEKTNETYGKLEAVQYKTQVLAAFFQRRWPGSTNYYIKVRAGDNKYMHLKVFNGPWKFRNTDRGADRVLTGYQVDKNKDDELTGF

SEQ ID NO: 144 - protein, 112 aa, Artificial Sequence
  Synthetic Polypeptide
MIPRGLSEAKPATPEIQEIVDKVKPQLEEKTNETYGKLEAVQYKTQVLANAFQRRWPGSTNYYIKVRAGDNKYMHLKVFNGPWKFRNTDRGADRVLTGYQVDKNKDDELTGF

SEQ ID NO: 145 - protein, 112 aa, Artificial Sequence
  Synthetic Polypeptide
MIPRGLSEAKPATPEIQEIVDKVKPQLEEKTNETYGKLEAVQYKTQVLANFAQRRWPGSTNYYIKVRAGDNKYMHLKVFNGPWKFRNTDRGADRVLTGYQVDKNKDDELTGF

SEQ ID NO: 146 - protein, 112 aa, Artificial Sequence
  Synthetic Polypeptide
MIPRGLSEAKPATPEIQEIVDKVKPQLEEKTNETYGKLEAVQYKTQVLANFFARRWPGSTNYYIKVRAGDNKYMHLKVFNGPWKFRNTDRGADRVLTGYQVDKNKDDELTGF

SEQ ID NO: 147 - protein, 112 aa, Artificial Sequence
  Synthetic Polypeptide
MIPRGLSEAKPATPEIQEIVDKVKPQLEEKTNETYGKLEAVQYKTQVLANFFQARWPGSTNYYIKVRAGDNKYMHLKVFNGPWKFRNTDRGADRVLTGYQVDKNKDDELTGF

SEQ ID NO: 148 - protein, 112 aa, Artificial Sequence
  Synthetic Polypeptide
MIPRGLSEAKPATPEIQEIVDKVKPQLEEKTNETYGKLEAVQYKTQVLANFFQRAWPGSTNYYIKVRAGDNKYMHLKVFNGPWKFRNTDRGADRVLTGYQVDKNKDDELTGF

SEQ ID NO: 149 - protein, 112 aa, Artificial Sequence
  Synthetic Polypeptide
MIPRGLSEAKPATPEIQEIVDKVKPQLEEKTNETYGKLEAVQYKTQVLANFFQRRAPGSTNYYIKVRAGDNKYMHLKVFNGPWKFRNTDRGADRVLTGYQVDKNKDDELTGF

SEQ ID NO: 150 - protein, 112 aa, Artificial Sequence
  Synthetic Polypeptide
MIPRGLSEAKPATPEIQEIVDKVKPQLEEKTNETYGKLEAVQYKTQVLANFFQRRWAGSTNYYIKVRAGDNKYMHLKVFNGPWKFRNTDRGADRVLTGYQVDKNKDDELTGF

SEQ ID NO: 151 - protein, 112 aa, Artificial Sequence
  Synthetic Polypeptide
MIPRGLSEAKPATPEIQEIVDKVKPQLEEKTNETYGKLEAVQYKTQVLANFFQRRWPASTNYYIKVRAGDNKYMHLKVFNGPWKFRNTDRGADRVLTGYQVDKNKDDELTGF

SEQ ID NO: 152 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPAKFRNTDRGADRVLTGYQVDKNKDDELTGF

SEQ ID NO: 153 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWAFRNTDRGADRVLTGYQVDKNKDDELTGF

SEQ ID NO: 154 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKARNTDRGADRVLTGYQVDKNKDDELTGF

SEQ ID NO: 155 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFANTDRGADRVLTGYQVDKNKDDELTGF

SEQ ID NO: 156 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRATDRGADRVLTGYQVDKNKDDELTGF

SEQ ID NO: 157 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNADRGADRVLTGYQVDKNKDDELTGF

SEQ ID NO: 158 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTARGADRVLTGYQVDKNKDDELTGF

SEQ ID NO: 159 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDAGADRVLTGYQVDKNKDDELTGF

SEQ ID NO: 160 - protein, 112 aa, Artificial Sequence
  Synthetic Polypeptide
MIPRGLSEAKPATPEIQEIVDKVKPQLEEKTNETYGKLEAVQYKTQVLANFFQRRWPGSTNYYIKVRAGDNKYMHLKVFNGPWKFRNTDRAADRVLTGYQVDKNKDDELTGF

SEQ ID NO: 161 - protein, 36 aa, Artificial Sequence
  Synthetic Polypeptide
AEAAAKEAAAKEAAAKEAAAKEAAAKEAAAKEAAAK

SEQ ID NO: 162 - protein, 10 aa, Artificial Sequence
  Synthetic Polypeptide
EQKLISEEDL

SEQ ID NO: 163 - protein, 7 aa, Artificial Sequence
  Synthetic Polypeptide
  SITE (7)..(7): X may be G or S
ENLYFQX

SEQ ID NO: 164 - protein, 7 aa, Artificial Sequence
  Synthetic Polypeptide
ENLYFQG

SEQ ID NO: 165 - protein, 35 aa, Artificial Sequence
  Synthetic Polypeptide
GGGGSGGGGSGGGGSGGGGSGGGGSGGGGSGGGGS

SEQ ID NO: 166 - protein, 136 aa, Artificial Sequence
  Synthetic Polypeptide
  SITE (4)..(4): Xaa may be Gly, Ala, Val, Arg, Lys, Asp, or Glu
  misc_feature (50)..(70): Xaa can be any naturally occurring amino acid
  misc_feature (53)..(70): May be absent
  SITE (71)..(71): Xaa may be Gly, Ala, Val, Ser or Thr
  SITE (92)..(92): Xaa may be Arg, Lys, Asn, Gln, Ser, Thr
  SITE (93)..(93): Xaa may be Gly, Ala, Val, Ser or Thr
  SITE (94)..(94): Xaa may be Ala, Val, Ile, Leu, Gly or Pro
  misc_feature (95)..(115): Xaa can be any naturally occurring amino acid
  misc_feature (98)..(115): May be absent
  SITE (116)..(116): Xaa may be Gly, Ala, Val, Asp or Glu
  SITE (118)..(118): Xaa may be Ala, Val, Ile, Leu, Arg or Lys
MIPXGLSEAKPATPEIQEIVDKVKPQLEEKTNETYGKLEAVQYKTQVLAXXXXXXXXXXXXXXXXXXXXXXTNYYIKVRAGDNKYMHLKVFXXXXXXXXXXXXXXXXXXXXXXXXXDXVLTGYQVDKNKDDELTGF

SEQ ID NO: 167 - protein, 136 aa, Artificial Sequence
  Synthetic Polypeptide
  misc_feature (50)..(70): Xaa can be any naturally occurring amino acid
  misc_feature (53)..(70): May be absent
  misc_feature (95)..(115): Xaa can be any naturally occurring amino acid
  misc_feature (98)..(115): May be absent
MIPRGLSEAKPATPEIQEIVDKVKPQLEEKTNETYGKLEAVQYKTQVLAXXXXXXXXXXXXXXXXXXXXXSTNYYIKVRAGDNKYMHLKVFNGPXXXXXXXXXXXXXXXXXXXXXADRVLTGYQVDKNKDDELTGF

SEQ ID NO: 168 - protein, 6 aa, Artificial Sequence
  Synthetic Polypeptide
HHHHHH

SEQ ID NO: 169 - protein, 18 aa, Artificial Sequence
  Synthetic Polypeptide
  misc_feature (11)..(13): Xaa can be any naturally occurring amino acid
  misc_feature (16)..(17): Xaa can be any naturally occurring amino acid
GIWASKWPGFXXXSKXXQ

SEQ ID NO: 170 - protein, 18 aa, Artificial Sequence
  Synthetic Polypeptide
  misc_feature (11)..(13): Xaa can be any naturally occurring amino acid
  misc_feature (16)..(17): Xaa can be any naturally occurring amino acid
GYYQRRWPGWXXXAGXXW

SEQ ID NO: 171 - protein, 18 aa, Artificial Sequence
  Synthetic Polypeptide
  misc_feature (11)..(13): Xaa can be any naturally occurring amino acid
  misc_feature (16)..(17): Xaa can be any naturally occurring amino acid
GFWAAAWPGFXXXSYXXA

SEQ ID NO: 172 - protein, 18 aa, Artificial Sequence
  Synthetic Polypeptide
  misc_feature (11)..(13): Xaa can be any naturally occurring amino acid
  misc_feature (16)..(17): Xaa can be any naturally occurring amino acid
GHWASKWPGFXXXSKXXQ

SEQ ID NO: 173 - protein, 18 aa, Artificial Sequence
  Synthetic Polypeptide
  misc_feature (12)..(17): Xaa can be any naturally occurring amino acid
NWYQQRWPGWHXXXXXXG

SEQ ID NO: 174 - protein, 18 aa, Artificial Sequence
  Synthetic Polypeptide
  misc_feature (12)..(17): Xaa can be any naturally occurring amino acid
NFFQRRWPGWKXXXXXXG

SEQ ID NO: 175 - protein, 18 aa, Artificial Sequence
  Synthetic Polypeptide
  misc_feature (5)..(5): Xaa can be any naturally occurring amino acid
  misc_feature (11)..(12): Xaa can be any naturally occurring amino acid
  misc_feature (15)..(18): Xaa can be any naturally occurring amino acid
EWWQXRWPGYXXHKXXXX

SEQ ID NO: 176 - protein, 18 aa, Artificial Sequence
  Synthetic Polypeptide
  misc_feature (5)..(5): Xaa can be any naturally occurring amino acid
  misc_feature (11)..(12): Xaa can be any naturally occurring amino acid
  misc_feature (15)..(18): Xaa can be any naturally occurring amino acid
DFYQXKWPNFXXKQXXXX

SEQ ID NO: 177 - protein, 18 aa, Artificial Sequence
  Synthetic Polypeptide
  misc_feature (5)..(5): Xaa can be any naturally occurring amino acid
  misc_feature (11)..(12): Xaa can be any naturally occurring amino acid
  misc_feature (15)..(18): Xaa can be any naturally occurring amino acid
EWWQXHWPHWXXAVXXXX

SEQ ID NO: 178 - protein, 18 aa, Artificial Sequence
  Synthetic Polypeptide
  misc_feature (5)..(5): Xaa can be any naturally occurring amino acid
  misc_feature (11)..(12): Xaa can be any naturally occurring amino acid
  misc_feature (15)..(18): Xaa can be any naturally occurring amino acid
EWWQXRWPGYXXQKXXXX

SEQ ID NO: 179 - protein, 10 aa, Artificial Sequence
  Synthetic Polypeptide
  SITE (2)..(2): Xaa may be Trp or Phe
  SITE (3)..(3): Xaa may be Trp or Phe
NXXQQRRWPG

SEQ ID NO: 180 - protein, 9 aa, Artificial Sequence
  Synthetic Polypeptide
  SITE (1)..(1): Xaa may be D, G, N, or V
  SITE (2)..(2): Xaa may be W, Y, H, or F
  SITE (3)..(3): Xaa may be W, Y, G, W, or F
  SITE (4)..(4): Xaa may be Q, A, or P
  SITE (5)..(5): Xaa may be A, Q, E, R, or S
  SITE (6)..(6): Xaa may be K, R, or Y
  SITE (7)..(7): Xaa may be W or Q
  SITE (8)..(8): Xaa may be P or H
  SITE (9)..(9): Xaa may be H, G, or Q
XXXXXXXXX

SEQ ID NO: 181 - protein, 9 aa, Artificial Sequence
  Synthetic Polypeptide
  SITE (1)..(1): Xaa may be Y, F, W, or N
  SITE (2)..(2): Xaa may be K, P, H, A, or T
  SITE (3)..(3): Xaa may be V, N, G, Q, A, or F
  SITE (4)..(4): Xaa may be H, T, Y, W, K, V, or R
  SITE (5)..(5): Xaa may be Q, S, G, P, or N
  SITE (6)..(6): Xaa may be S, Y, E, L, K, or T
  SITE (7)..(7): Xaa may be S, D, V, or K
  SITE (8)..(8): Xaa may be G, L, S, P, H, D, or R
  SITE (9)..(9): Xaa may be G, Q, E, or A
XXXXXXXXX

(19) United States
(12) Patent Application Publication
(10) Pub. No.: US 2023/0272011 A1
(43) Pub. Date: Aug. 31, 2023

(54) SERUM ALBUMIN-BINDING POLYPEPTIDES

(71) Applicant: Avacta Life Sciences Limited, Wetherby (GB)

(72) Inventors: Emma Jenkins, Stansted (GB); Estelle Adam, Hitchin (GB); Emma Stanley, Hitchin (GB); Amrik Basran, Cambridge (GB); Matthew P. Vincent, Amesbury, MA (US); Bruno Gomes, Vila do Conde (PT)

(73) Assignee: Avacta Life Sciences Limited, Wetherby (GB)

(21) Appl. No.: 18/018,208

(22) PCT Filed: Jul. 30, 2021
(86) PCT No.: PCT/EP2021/071421

§ 371 Date: Jan. 26, 2023

Related U.S. Application Data
(60) Provisional application No. 63/059,026, filed on Jul. 30, 2020.

(51) Int. Cl. C07K 14/00; C07K 14/575; C07K 14/505; C07K 14/605; C12N 15/70

(52) U.S. Cl. CPC C07K 14/001; C07K 14/57545; C07K 14/505; C07K 14/605; C12N 15/70; C07K 2319/00; C12N 2800/101

(57) ABSTRACT

Provided herein, in some embodiments, are recombinantly engineered variant of stefin polypeptides (AFFIMER® polypeptides) that binds to human serum albumin and extends the half-life of the polypeptides. Also provided herein, in some embodiments, are composition containing the polypeptides, methods of using the polypeptides, and methods of producing the polypeptides.

### RELATED APPLICATION

This application claims the benefit under 35 U.S.C. § 119(e) of U.S. 63/059,026, filed Jul. 30, 2021, which is incorporated by reference herein in its entirety.

### BACKGROUND

Human serum albumin is the primary protein present in human blood plasma. It presents approximately 50% of the total protein content in healthy humans. Human albumin is a small globular protein (molecular weight: 66.5 kDa), consisting of a single chain of 585 amino acids organized in three repeated homolog domains (sites I, II, and III). Each domain comprises two separate sub-domains (A and B). Human serum albumin is a vehicle for a host of small molecules and proteins, regulates oncotic pressure, and performs the majority of antioxidation in the body. Often, it is used to enhance drug delivery and in maintaining cell culture.

### SUMMARY

Provided herein, in some aspects, is a half-life extension platform based on recombinantly engineered variant of stefin polypeptides (AFFIMER® polypeptides) that bind to serum albumin (e.g., human serum albumin (HSA)). A range of human serum albumin-binding AFFIMER® polypeptides (referred to as anti-HSA AFFIMER® polypeptides), with a range of binding affinities, has been developed. These anti-HSA AFFIMER® polypeptides cross-react with other species such as mouse and cynomolgous monkey. These polypeptides have been shown in in vivo pharmacokinetic (PK) studies to extend, in a controlled manner, the serum half-life of any other AFFIMER® polypeptides to which it is conjugated (e.g., as a single genetic fusion) that can be made, for example, in bacterial cells (e.g., Escherichia coli). The serum albumin-binding AFFIMER® polypeptides provided herein can also be used to extend the half-life of other polypeptides, such as therapeutic proteins.

Thus, some aspects of the present disclosure provide AFFIMER® polypeptides that bind to serum albumin, such as human serum albumin (HSA). In some embodiments, the polypeptides bind to HSA with a K_{d} of 1×10⁻⁶ M or less at pH 7.4, and at pH 6 bind to HSA with a K_{d} that is at least half a log less than the K_{d} for binding to HSA at pH 7.4.

In some embodiments, the polypeptides bind to HSA with a K_{d} of 1×10⁻⁷ M or less at pH 7.4, a K_{d} of 1×10⁻⁸ M or less at pH 7.4, or K_{d} of 1×10⁻⁹ M or less at pH 7.4. In some embodiments, the polypeptides at pH 6 bind to HSA with a K_{d} that is at least one log less than the K_{d} for binding to HSA at pH 7.4, at least 1.5 logs less than the K_{d} for binding to HSA at pH 7.4, at least 2 logs less than the K_{d} for binding to HSA at pH 7.4, or at least 2.5 log less than the K_{d} for binding to HSA at pH 7.4

In some embodiments, the polypeptides have a serum half-life in human patients of greater than 10 hours, greater than 24 hours, greater than 48 hours, greater than 72 hours, greater than 96 hours, greater than 120 hours, greater than 144 hours, greater than 168 hours, greater than 192 hours, greater than 216 hours, greater than 240 hours, greater than 264 hours, greater than 288 hours, greater than 312 hours, greater than 336 hours or, greater than 360 hours.

In some embodiments, the polypeptides have a serum half-life in human patients of greater than 50%, greater than 60%, greater than 70%, or greater than 80% of the serum half-life of HSA.

In some embodiments, the polypeptides comprise an amino acid sequence represented in general formula (I): FR1-(Xaa)ₙ-FR2-(Xaa)ₘ-FR3 (I), wherein FR1 is an amino acid sequence having at least 70% identity to MIPGGLSEAK PATPEIQEIV DKVKPQLEEK TNETYGKLEA VQYKTQVLA (SEQ ID NO: 1); FR2 is an amino acid sequence having at least 70% identity to GTNYYIKVRA GDNKYMHLKV FKSL (SEQ ID NO: 2); FR3 is an amino acid sequence having at least 70% identity to EDLVLTGYQV DKNKDDELTG F (SEQ ID NO: 3); and Xaa, individually for each occurrence, is an amino acid, n is an integer from 3 to 20, and m is an integer from 3 to 20. In some embodiments, FR1 has at least 75%, at least 80%, at least 85%, at least 90%, or at least 95% identity to SEQ ID NO: 1. In some embodiments, FR1 comprises the amino acid sequence of SEQ ID NO: 1. In some embodiments, FR2 has at least 75%, at least 80%, at least 85%, at least 90%, or at least 95% identity to SEQ ID NO: 2. In some embodiments, FR2 comprises the amino acid sequence of SEQ ID NO: 2. In some embodiments, FR3 has at least 75%, at least 80%, at least 85%, at least 90%, or at least 95% identity to SEQ ID NO: 3. In some embodiments, FR3 comprises the amino acid sequence of SEQ ID NO: 3.

In some embodiments, the amino acid sequence of an AFFIMER® polypeptide provided herein is represented in general formula (II): MIP-Xaa1-GLSEAKPATPEIQEIVDKVKPQLEEKTNETYGKLEAVQYKTQVLA-(Xaa)ₙ-Xaa2-TNYYIKVRAGDNKYMHLKVF-Xaa3-Xaa4-Xaa5-(Xaa)ₘ-Xaa6-D-Xaa7-VLTGYQVDKNKDDELTGF (SEQ ID NO: 166) (II), wherein Xaa, individually for each occurrence, is an amino acid; n is an integer from 3 to 20, and m is an integer from 3 to 20; Xaa1 is Gly, Ala, Val, Arg, Lys, Asp, or Glu; Xaa2 is Gly, Ala, Val, Ser or Thr; Xaa3 is Arg, Lys, Asn, Gln, Ser, Thr; Xaa4 is Gly, Ala, Val, Ser or Thr; Xaa5 is Ala, Val, Ile, Leu, Gly or Pro; Xaa6 is Gly, Ala, Val, Asp or Glu; and Xaa7 is Ala, Val, Ile, Leu, Arg or Lys.

In some embodiments, the amino acid sequence of an AFFIMER® polypeptide provided herein is represented in general formula (III):

(SEQ ID NO: 167)
MIPRGLSEAKPATPEIQEIVDKVKPQLEEKTNETYGKLEAVQYKT
QVLA-(Xaa)n-STNYYIKVRAGDNKYMHLKVFNGP-(Xaa)m-ADR
VLTGYQVDKNKDDELTGF,
(III)

wherein Xaa, individually for each occurrence, is an amino acid; n is an integer from 3 to 20, and m is an integer from 3 to 20.

In some embodiments, (Xaa)ₙ is represented by formula (IV):
aa1-aa2-aa3-aa4-aa5-aa6-aa7-aa8-aa9 (SEQ ID NO: 180) (IV), wherein aa1 is an amino acid selected from D, G, N, and V; aa2 is an amino acid selected from W, Y, H, and F; aa3 is an amino acid selected from W, Y, G, W, and F; aa4 is an amino acid selected from Q, A, and P; aa5 is an amino acid selected from A, Q, E, R, and S; aa6 is an amino acid selected from K, R, and Y; aa7 is an amino acid selected from W and Q; aa8 is an amino acid selected from P and H; aa9 is an amino acid selected from H, G, and Q.

In some embodiments, (Xaa)ₙ is an amino acid sequence having at least 80% or at least 90% identity to the amino acid sequence of any one of SEQ ID NOS: 4-55. In some embodiments, (Xaa)ₙ is the amino acid sequence of any one of SEQ ID NOS: 4-55. In some embodiments, (Xaa)ₙ is an amino acid sequence having at least 80% or at least 90% identity to the amino acid sequence of any one of SEQ ID NOS: 22, 24, 26, 35, 40, 41, and 45. In some embodiments, (Xaa)ₙ is an amino acid sequence selected from an amino acid sequence of any one of SEQ ID NOS: 22, 24, 26, 35, 40, 41, and 45.

In some embodiments, (Xaa)ₘ is represented by formula (IV):
aa1-aa2-aa3-aa4-aa5-aa6-aa7-aa8-aa9 (SEQ ID NO: 181) (IV), wherein aa1 is an amino acid selected from Y, F, W, and N; aa2 is an amino acid selected from K, P, H, A, and T; aa3 is an amino acid selected from V, N, G, Q, A, and F; aa4 is an amino acid selected from H, T, Y, W, K, V, and R; aa5 is an amino acid selected from Q, S, G, P, and N; aa6 is an amino acid selected from S, Y, E, L, K, and T; aa7 is an amino acid selected from S, D, V, and K; aa8 is an amino acid selected from G, L, S, P, H, D, and R; aa9 is an amino acid selected from G, Q, E, and A.

In some embodiments, (Xaa)ₘ is an amino acid sequence having at least 80% or at least 90% identity to the amino acid sequence of any one of SEQ ID NOS: 57-108. In some embodiments, (Xaa)ₘ is the amino acid sequence of any one of SEQ ID NOS: 57-108. In some embodiments, (Xaa)ₘ is an amino acid sequence having at least 80% or at least 90% identity to the amino acid sequence of any one of SEQ ID NOS: 75, 77, 79, 88, 93, 94, and 98. In some embodiments, (Xaa)ₘ is an amino acid sequence selected from an amino acid sequence of any one of SEQ ID NOS: 75, 77, 79, 88, 93, 94, and 98.

In some embodiments, the amino acid sequence has at least 70% identity to an amino acid sequence of any one of SEQ ID NOS: 110-116 and 138. In some embodiments, the amino acid sequence comprises an amino acid sequence of any one of SEQ ID NOS: 110-116 and 138.

In some embodiments, (Xaa)ₙ is represented by formula (IV):
aa1-aa2-aa3-aa4-aa5-aa6-aa7-aa8-aa9 (IV), wherein aa1 is an amino acid with a neutral polar hydrophilic side chain; aa2 is an amino acid with a neutral nonpolar hydrophobic side chain; aa3 is an amino acid with a neutral nonpolar hydrophobic side chain; aa4 is an amino acid with a neutral polar hydrophilic side chain; aa5 is an amino acid with a positively charged polar hydrophilic side chain; aa6 is an amino acid with a positively charged polar hydrophilic side chain; aa7 is an amino acid with a neutral nonpolar hydrophobic side chain; aa8 is an amino acid with a neutral nonpolar hydrophobic side chain; and aa9 is an amino acid with a neutral nonpolar hydrophilic side chain.

In some embodiments, (Xaa)ₘ is represented by formula (IV):
aa1-aa2-aa3-aa4-aa5-aa6-aa7-aa8-aa9 (IV), wherein aa1 is an amino acid with a neutral nonpolar hydrophobic side chain; aa2 is an amino acid with a positively charged polar hydrophilic side chain; aa3 is an amino acid with a neutral nonpolar hydrophobic side chain; aa4 is an amino acid with a positively charged polar hydrophilic side chain; aa5 is an amino acid with a neutral polar hydrophilic side chain; aa6 is an amino acid with a neutral polar hydrophilic side chain; aa7 is an amino acid with a negatively charged polar hydrophilic side chain; aa8 is an amino acid with a positively charged polar hydrophilic side chain; and aa9 is an amino acid with a neutral nonpolar hydrophilic side chain.

In some embodiments, the amino acid with the neutral nonpolar hydrophilic side chain is selected from cysteine (C or Cys) and glycine (G or Gly); the amino acid with the neutral nonpolar hydrophobic side chain is selected from alanine (A or Ala), isoleucine (I or Ile), leucine (L or Leu), methionine (M or Met), phenylalanine (F or Phe), proline (P or Pro), tryptophan (W or Trp), and valine (V or Val); the amino acid with the neutral polar hydrophilic side chain is selected from asparagine (N or Asn), glutamine (Q or Gln), serine (S or Ser), threonine (T or Thr), and tyrosine (Y or Tyr); the amino acid with the positively charged polar hydrophilic side chain is selected from arginine (R or Arg), histidine (H or His), and lysine (K or Lys); and the amino acid with the negatively charged polar hydrophilic side chain is selected from aspartate (D or Asp) and glutamate (E or Glu).

In some embodiments, the amino acid sequence of an AFFIMER® polypeptide provided herein is represented in general formula (III):

(SEQ ID NO: 167)
MIPRGLSEAKPATPEIQEIVDKVKPQLEEKTNETYGKLEAVQYKT
QVLA-(Xaa)n-STNYYIKVRAGDNKYMHLKVFNGP-(Xaa)m-ADR
VLTGYQVDKNKDDELTGF,
(III)

wherein (Xaa)ₙ is an amino acid sequence selected from an amino acid sequence of any one of SEQ ID NOS: 4-55 or any one of SEQ ID NOS: 22, 24, 26, 35, 40, 41, and 45 and/or (Xaa)ₘ is an amino acid sequence selected from an amino acid sequence of any one of SEQ ID NOS: 57-108 or any one of SEQ ID NOS: 75, 77, 79, 88, 93, 94, and 98.

In some embodiments, the amino acid sequence of the polypeptides comprises a cysteine optionally available for chemical conjugation, and optionally wherein the cysteine is located at the C-terminal end or the N-terminal end of the polypeptide.

In some embodiments, the polypeptides further comprise a heterologous polypeptide covalently linked through an amide bond to form a contiguous fusion protein.

In some embodiments, the heterologous polypeptide comprises a therapeutic polypeptide.

In some embodiments, the therapeutic polypeptide is selected from the group consisting of hormones, cytokines, chemokines, growth factors, hemostasis active polypeptides, enzymes, and toxins. In some embodiments, the therapeutic polypeptide is an antagonist of hormones, cytokines, chemokines, growth factors, hemostasis active polypeptides, enzymes, or toxins.

In some embodiments, the therapeutic polypeptide is selected from the group consisting of receptor traps and receptor ligands. In some embodiments, the therapeutic polypeptide is an antagonist of receptor traps or receptor ligands.

In some embodiments, the therapeutic polypeptide sequence is selected from the group consisting of angiogenic agents and anti-angiogenic agents. In some embodiments, the therapeutic polypeptide is an antagonist of angiogenic agents or anti-angiogenic agents.

In some embodiments, the therapeutic polypeptide sequence is a neurotransmitter, for example, Neuropeptide Y.

In some embodiments, the therapeutic polypeptide sequence is an erythropoiesis-stimulating agent, for example, erythropoietin or an erythropoietin mimetic.

In some embodiments, the therapeutic polypeptide is an incretin. For example, the incretin may be glucagon, gastric inhibitory peptide (GIP), glucagon-like peptide-1 (GLP-1), glucagon-like peptide-2 (GLP-2), peptide YY (PYY), or oxyntomodulin (OXM).

To further illustrate, in some embodiments the therapeutic proteins of the present invention include, in addition to at least one HSA binding AFFIMER® sequence, an erythropoietin (EPO) polypeptide sequence, such as shown in SEQ ID NO:133

SEQ ID NO: 133
APPRLICDSR VLERYLLEAK EAENITTGCA EHCSLNENIT
VPDTKVNFYA WKRMEVGQQA VEVWQGLALL SEAVLRGQAL
LVNSSQPWEP LQLHVDKAVS GLRSLTTLLR ALGAQEAISP
PDAASAAPLR TITADTFRKL FRVYSNFLRG KLKLYTGEAC
RTGD

The polypeptide sequence for an exemplary XT/EPO fusion is shown as SEQ ID NO: 134 where the first underlined sequence is a secretion signal sequence and the second underlined sequence is a (G₄S)ₙ linked EPO polypeptide sequence:

SEQ ID NO: 134
MPLLLLLPLL WAGALAIPRGLSEA KPATPEIQEI VDKVKPQLEE
KTNETYGKLE AVQYKTQVLA NFFQRRWPGS TNYYIKVRAG
DNKYMHLKVF NGPWKFRNTD RGADRVLTGY QVDKNKDDEL
TGFAAAGGRA EQKLISEEDL GAAGGGGSGG GGSGGGGSGG
GGSAPTSSSA PPRLICDSRV LERYLLEAKE AENITTGCAE
HCSLNENITV PDTKVNFYAW KRMEVGQQAV EVWQGLALLS
EAVLRGQALL VNSSQPWEPL QLHVDKAVSG LRSLITLLRA
LGAQEAISPP DAASAAPLRT ITADTFRKLF RVYSNFLRGK
LKLYTGEACR TGD

In some embodiments, a variant sequence for EPO is used, in which one or more amino acid residues which can serves as sites for glycosylation are been replaced with an amino acid residue which does not serve as a site for glycosylation. For instance, one or more of amino acid residues Asn24, Asn38, Asn83 and Ser126 of SEQ ID NO: 133 can be altered, such as with an amino acid residue other than Asn or Ser, e.g., replaced with Ala. The polypeptide sequence for an exemplary XT/variant EPO fusion is shown as SEQ ID NO: 135, where the first underlined sequence is a secretion signal sequence and the second underlined sequence is a (G₄S)ₙ linked variant EPO polypeptide sequence:

SEQ ID NO: 135
MPLLLLLPLL WAGALAIPRGLSEA KPATPEIQEI VDKVKPQLEE
KTNETYGKLE AVQYKTQVLA NFFQRRWPGS TNYYIKVRAG
DNKYMHLKVF NGPWKFRNTD RGADRVLTGY QVDKNKDDEL
TGFAAAGGRA EQKLISEEDL GAAGGGGSGG GGSGGGGSGG
GGSAPTSSSA PPRLICDSRV LERYLLEAKE AEAITTGCAE
HCSLNEAITV PDTKVNFYAW KRMEVGQQAV EVWQGLALLS
EAVLRGQALL VASSQPWEPL QLHVDKAVSG LRSLTTLLRA
LGAQEAISPP DAASAAPLRT ITADTFRKLF RVYSNFLRGK
LKLYTGEACR TGD

As an example of an incretin-XT fusion, the GLP-1 analogs used for dulaglutide or exendin-4 can be used to create a fusion protein with an HSA binding AFFIMER® sequence, such as shown in SEQ ID NO: 136 or 137, where the first underlined sequence is a secretion signal sequence and the second underlined sequence is a (G₄S)ₙ linked separating the GLP-1 variant polypeptide sequence and the HSA binding AFFIMER® polypeptide sequence:

SEQ ID NO: 136
MPLLLLLPLL WAGALAHGEG TFTSDVSSYL LEEQAAKEFI
AWLVKGGGGS GGGGSGGGGS GGGGSIPRGL SEAKPATPEI
QEIVDKVKPQ LEEKTNETYG KLEAVQYKTQ VLANFFQRRW
PGSTNYYIKV RAGDNKYMHL KVFNGPWKFR NTDRGADRVL
TGYQVDKNKD DELTGFAAAG GRAEQKLISE EDLGAA
SEQ ID NO: 137
MPLLLLLPLL WAGALAHGEG TFTSDLSKQM EEEAVRLFIE
WLKNGGPSSG APPPSHGEGT FTSDVSSYLL EEQAAKEFIA
WLVKGGGGSG GGGSGGGGSG GGGSIPRGLS EAKPATPEIQ
EIVDKVKPQL EEKTNETYGK LEAVQYKTQV LANFFQRRWP
GSTNYYIKVR AGDNKYMHLK VFNGPWKFRN TDRGADRVLT
GYQVDKNKDD ELIGFAAAGG RAEQKLISEE DLGAA

In some embodiments, the polypeptides extend the serum half-life of the heterologous polypeptide in vivo. For example, the heterologous polypeptide may have an extended half-life that is at least 2-fold, at least 5-fold, at least 10-fold, at least 20-fold, or at least 30-fold greater than the half-life of the heterologous polypeptide not linked to the AFFIMER® polypeptide.

Other aspects of the present disclosure provide pharmaceutical preparations, e.g., for therapeutic use in a human patient, comprising any of the AFFIMER® polypeptides described herein, and a pharmaceutically acceptable excipient (e.g., carrier, buffer, and/or salt, etc.).

Further aspects of the present disclosure provide polynucleotides comprising a sequence encoding the AFFIMER® polypeptides described herein.

In some embodiments, the sequence encoding a polypeptide is operably linked to a transcriptional regulatory sequence. The transcriptional regulatory sequence may be, for example, a promoter or an enhancer. Other transcriptional regulatory sequences are contemplated herein.

In some embodiments, a polynucleotide further comprises an origin of replication, a minichromosome maintenance element (MME), and/or a nuclear localization element. In some embodiments, a polynucleotide further comprise a polyadenylation signal sequence operably linked and transcribed with the sequence encoding the polypeptide. In some embodiments, a polynucleotide further comprises at least one intronic sequence. In some embodiments, a polynucleotide further comprises at least one ribosome binding site transcribed with the sequence encoding the polypeptide.

In some embodiments, a polynucleotide is a deoxyribonucleic acid (DNA). In some embodiments, a polynucleotide is a ribonucleic acid (RNA).

Further aspects of the present disclosure provide viral vectors, plasmids, and/or minicircles comprising the AFFIMER® polypeptides described herein.

Additional aspects of the present disclosure provide methods that comprise administering to a subject having an autoimmune disease a therapeutically effective amount of the AFFIMER® polypeptides described herein.

In some embodiments, the disclosure provides a fusion protein comprising any one of the polypeptides described herein. In some embodiments, the fusion protein further comprises a linker. In some embodiments, the linker is a rigid linker. In some embodiments, the rigid linker comprises the sequence of SEQ ID NO: 161. In some embodiments, the linker is a flexible linker. In some embodiments, the flexible linker comprises the sequence of SEQ ID NO: 165.

In some embodiments, the fusion protein comprises two of any of the polypeptides described herein.

In some embodiments, the fusion protein further comprises a therapeutic molecule. In some embodiments, the therapeutic molecule is a therapeutic polypeptide. In some embodiments, the therapeutic polypeptide is selected from hormones, cytokines, chemokines, growth factors, hemostasis active polypeptides, enzymes, and toxins, or is selected from antagonists of hormones, cytokines, chemokines, growth factors, hemostasis active polypeptides, enzymes, and toxins.

In some embodiments, the polypeptide comprises the amino acid sequence of SEQ ID NO: 110. In some embodiments, the polypeptide comprises the amino acid sequence of SEQ ID NO: 113. In some embodiments, the polypeptide comprises the amino acid sequence of SEQ ID NO: 116.

### BRIEF DESCRIPTION OF THE DRAWINGS

FIG. 1 Alignment of AFFIMER® nine amino acid binding loops (loop 2 and 4) sequences selected using phage display. The sequences, from left to right and top to bottom, correspond to SEQ ID NOs: 22, 75, 45, 98, 26, 79, 48, 101, 23, 76, 40, 93, 41, 94, 24, 77, 33, 86, 36, 89, 35, 88, 38, 91, 50, 103, 56, and 109.

FIG. 2 AFFIMER® binding loop sequence families of similar motifs from serum albumin phage selections. The sequences, from left to right and top to bottom, correspond to SEQ ID NOs: 169-178.

FIGS. 3A and 3B SEC-HPLC (FIG. 3A) and SDS-PAGE (FIG. 3B) analysis of purified monomeric serum albumin binding AFFIMER® polypeptides.

FIGS. 4A-4D Octet kinetic binding analysis of purified serum albumin binding AFFIMER® polypeptides to different species of serum albumin at pH 6.0 and pH 7.4. HSA-20 (FIG. 4A), HSA-31 (FIG. 4B), HSA-36 (FIG. 4C), and HSA-41 (FIG. 4D) are shown.

FIG. 5 BIACORE™ kinetic analysis of purified serum albumin binding AFFIMER® proteins to different species of serum albumin at pH 6.0 and pH 7.4.

FIG. 6 AFFIMER® polypeptide binding ELISA to serum albumin from different species at pH 7.4.

FIG. 7 AFFIMER® polypeptide binding ELISA to serum albumin from different species at pH 6.0.

FIG. 8 Pharmacokinetic profile of five lead serum albumin binding AFFIMER® polypeptides in mouse.

FIGS. 9A and 9B Octet analysis of C-terminally His tag cleaved AFFIMER® lead clones at pH 7.4 (FIG. 9A) and pH 6.0 (FIG. 9B).

FIG. 10 Pharmacokinetics profile of C-terminal His tag cleaved serum albumin binding AFFIMER® polypeptide in mouse.

FIG. 11 BIACORE™ adjusted sensorgram demonstrating that FcRn binding of HSA is unaffected by the presence of a serum albumin binding AFFIMER® polypeptide.

FIG. 12A Schematic representation of a PD-L1/serum albumin binding in-line fusion (ILF) AFFIMER® protein.

FIG. 12B SEC-HPLC chromatograms of PD-L1/serum albumin binding ILF AFFIMER® proteins following purification.

FIG. 13 Schematic representation of PD-L1/serum albumin binding trimer ILF AFFIMER® proteins.

FIG. 14 Production and SDS-PAGE analysis of purified PD-L1/serum albumin binding trimer ILF AFFIMER® proteins.

FIG. 15 BIACORE™ kinetic analysis showing ILF AFFIMER® trimers retain binding to both PD-L1 target antigen and serum albumin.

FIG. 16 Graph showing half-life extended AFFIMER® ILF trimers binding to human PD-L1 by ELISA and exhibiting similar binding to the parental molecule AVA04-251.

FIG. 17 Graph showing the potency of half-life extended ILF AFFIMER® polypeptides is similar to the parental molecule in the PD-1/PD-L1 blockade Bioassay (PROMEGA®).

FIG. 18 Graph showing the half-life extended ILF AFFIMER® polypeptides binding to human serum albumin binding is equivalent by ELISA at pH 7.4.

FIG. 19 Mixed lymphocyte reaction (MLR) showing ILF trimer half-life extended AFFIMER® polypeptide (AVA04-251 XT14) is functional and retains potency when formatted compared to the parental molecule.

FIG. 20 Pharmacokinetic profile of ILF half-life extended trimers in mouse.

FIGS. 21A-21C In vivo efficacy of an ILF AVA04-251 XT14 in an A375 xenograft model. Individual traces over time are shown in FIG. 21A. FIG. 21B shows the results in FIG. 21A consolidated by group. FIG. 21C shows the tumor volume in each group.

FIG. 22 Expression and purification of AVA04-251 XT14-cys from E. coli.

FIG. 23 Pharmacokinetic profile of HSA-41 in double humanized neonatal Fc receptor (FcRn)/albumin mouse model.

FIG. 24 Pharmacokinetic profile of HSA-41, HSA-18 and HSA-31 in cynomolgus monkey.

FIG. 25A Anti-mouse PD-L1 AFFIMER® half-life extended trimer production and characterization.

FIG. 25B AVA04-182 XT20 K_{D} determination against mouse PD-L1 Fc using BIACORE™.

FIGS. 26A and 26B ELISA showing AVA04-182 XT20 binding to MSA at pH 7.4 (FIG. 26A) and 6.0 (FIG. 26B).

FIG. 26C mPD-L1 competition ELISA of both AVA04-182 and AVA04-182 XT20

FIG. 27 Pharmacokinetic profile of the AVA04-182 XT20 trimer, AVA04-182 Fc formatted AFFIMER® polypeptide in mice.

FIGS. 28A-28C Schematic (FIG. 28A) and characterization of AVA04-251 BH cys ILF dimer protein. FIG. 28B shows a purity analysis and FIG. 28C shows the SDS-PAGE analysis.

FIGS. 29A and 29B Evaluation of binding capacity of fluorescently labelled AFFIMER® polypeptides AVA04-251 BH cys800 (FIG. 29A) and AVA04-251 XT14 cys800 (FIG. 29B) compared to parental molecules using a binding ELISA to huPD-L1.

FIG. 30 Representative images of biodistribution of fluorescently labelled AFFIMER® anti-huPD-L1 polypeptides in two A375 melanoma xenograft models four hours post treatment.

FIG. 31A Image of crystals formed from HSA and anti-HSA AFFIMER® polypeptide HSA-41 complex.

FIG. 31B Calculated three-dimensional structures of the anti-HSA AFFIMER® polypeptide HSA-41 in complex with HSA derived from the crystallization of the protein complex.

FIGS. 31C and 31D Amino acid interactions between loop 2 (FIG. 31C) and loop 4 (FIG. 31D) residues of the AFFIMER® polypeptide at the interface of contact with HSA.

FIG. 32A Schematic of ILF homodimer HSA-41 formats.

FIG. 32B Table of K_{D} values for binding to HSA at pH 7.4 compared to monomer.

FIG. 32C BIACORE™ sensorgrams showing the avidity effects on HSA of the HSA-41 monomer when genetically linked to form a dimer.

FIGS. 33A-33C HSA-41 monomer incubations with serum albumin, SEC-HPLC characterization (FIG. 33A, 1:1 ratio; FIG. 33B, 1:2 ratio; FIG. 33C, 1:1 overlaid).

FIG. 34 SEC-HPLC characterization of HSA-41 in-line fusion (ILF) dimer incubations with serum albumin.

FIG. 35 Pharmacokinetic analysis of HSA-41 monomer and ILF dimer in C57BL/6 mice.

FIG. 36 Serum albumin Biacore kinetic analysis of HSA-41 loop 4 knockout mutants.

FIG. 37 Lead serum albumin binding AFFIMER® polypeptide epitope binning against HSA-41 using a Homogeneous Time Resolved Fluorescence (HTRF) assay.

FIGS. 38A and 38B SEC-HPLC and SDS-PAGE characterization of HSA-41 free C-terminal cysteine format (CQ).

FIG. 39 Biacore Kinetic Analysis for HSA-41 free C-terminal cysteine format (CQ) binding to HSA at pH7.4.

FIG. 40 Quality control analysis (purity) of AVA04-251 XT ILF with HSA-18 half-life extending AFFIMER® polypeptide (two different formats: XT60 and XT61).

FIGS. 41A and 41B Biacore kinetic analysis for the XT60 and XT61 ILF binding to HSA at pH7.4 (FIG. 41A) and pH6.0 (FIG. 41B).

FIG. 42 Binding ELISA for XT60 and XT61 ILF binding to HAS and MSA at pH7.4.

FIG. 43 XT60 and XT61 ILF Biacore kinetic analysis for binding to MSA at pH6.0.

FIG. 44 Biacore kinetic analysis for XT60 and XT61 ILF polypeptides binding to human PD-L1 Fc.

### DETAILED DESCRIPTION

The present disclosure is based on the generation of AFFIMER® polypeptides that bind to human serum albumin (HSA) to extend, in a controlled manner, the serum half-life of any other therapeutic molecules (e.g., therapeutic AFFIMER® polypeptide, protein, nucleic acid, or drug) to which it is conjugated. The experimental data herein demonstrate that the serum half-life of AFFIMER® polypeptide can be significantly increased by binding to albumin in vivo.

Based on naturally occurring proteins (cystatins) that have been engineered to stably display two loops that create a binding surface, the serum albumin-binding AFFIMER® polypeptides of the present disclosure provide a number of advantages over antibodies, antibody fragments, and other non-antibody molecule-binding proteins. One is the small size of the AFFIMER® polypeptide itself. In its monomeric form it is about 14 kDa, or 1/10th the size of an antibody. This small size gives greater potential for increased tissue penetration, particularly in poorly vascularized and/or fibrotic target tissues (like tumors). AFFIMER® polypeptides have a simple protein structure (versus multi-domain antibodies), and as the AFFIMER® polypeptides do not require disulfide bonds or other post-translational modifications for function, these polypeptides can be manufactured in prokaryotic and eukaryotic systems.

Using libraries of AFFIMER® polypeptides (such as the phage display techniques described in the appended examples) as well as site directed mutagenesis, AFFIMER® polypeptides can be generated with tunable binding kinetics with ideal ranges for therapeutic uses. For instance, the AFFIMER® polypeptides can have high affinity for HSA, such as single digit nanomolar or lower K_{d} for monomeric AFFIMER® polypeptides, and picomolar K_{d} and avidity in multi-valent formats. The AFFIMER® polypeptides can be generated with tight binding kinetics for HSA, such as slow K_{off} rates in the 10⁻⁴ to 10⁻⁵ (s−1) range, which benefits target tissue localization.

The serum albumin-binding AFFIMER® polypeptides of the present disclosure include AFFIMER® polypeptides with exquisite selectivity.

Moreover, the serum albumin-binding AFFIMER® polypeptides can be readily formatted, allowing formats such as Fc fusions, whole antibody fusions, and in-line multimers to be generated and manufactured with ease.

The lack of need for disulfide bonds and post-translational modifications also permit many embodiments of proteins including the serum albumin-binding AFFIMER® polypeptides to be delivered therapeutically by expression of gene delivery constructs that are introduced into the tissues of a patient, including formats where the protein is delivered systemically (such as expression from muscle tissue) or delivered locally (such as through intratumoral gene delivery).

An AFFIMER® polypeptide (also referred to simply as an AFFIMER®) is a small, highly stable polypeptide (e.g., protein) that is a recombinantly engineered variant of stefin polypeptides. Thus, the term “AFFIMER® polypeptide” may be used interchangeably herein with the term “recombinantly engineered variant of stefin polypeptide”. A stefin polypeptide is a subgroup of proteins in the cystatin superfamily—a family that encompasses proteins containing multiple cystatin-like sequences. The stefin subgroup of the cystatin family is relatively small (˜100 amino acids) single domain proteins. They receive no known post-translational modification, and lack disulfide bonds, suggesting that they will be able to fold identically in a wide range of extracellular and intracellular environments. Stefin A is a monomeric, single chain, single domain protein of 98 amino acids. The structure of stefin A has been solved, facilitating the rational mutation of stefin A into the AFFIMER® polypeptide. The only known biological activity of cystatins is the inhibition of cathepsin activity, has enabled exhaustively testing for residual biological activity of the engineered proteins.

AFFIMER® polypeptides display two peptide loops and an N-terminal sequence that can all be randomized to bind to desired target proteins with high affinity and specificity, in a similar manner to monoclonal antibodies. Stabilization of the two peptides by the stefin A protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the stefin A polypeptide sequence can be carried out, with such variations improving the properties of these affinity reagents, such as increase stability, make them robust across a range of temperatures and pH, for example. In some embodiments, an AFFIMER® polypeptide includes a sequence derived from stefin A, sharing substantial identify with a stefin A wild type sequence, such as human stefin A. In some embodiments, an AFFIMER® polypeptide has an amino acid sequence that shares at least 25%, 35%, 45%, 55% or 60% identity to the sequences corresponding to human stefin A. For example, an AFFIMER® polypeptide may have an amino acid sequence that shares at least 70%, at least 80%, at least 85%, at least 90%, at least 92%, at least 94%, at least 95% identity, e.g., where the sequence variations do not adversely affect the ability of the scaffold to bind to the desired target, and e.g., which do not restore or generate biological functions such as those that are possessed by wild type stefin A, but which are abolished in mutational changes described herein.

### Human Serum Albumin Binding AFFIMER® Polypeptides

One aspect of the disclosure provides AFFIMER® polypeptides that bind human serum albumin (HSA) (referred to as anti-HSA AFFIMER® polypeptides). Human serum albumin (HSA) is a protein encoded by the ALB gene. HSA is a 585 amino acid polypeptide (approx. 67 kDa) having a serum half-life of about 20 days and is primarily responsible for the maintenance of colloidal osmotic blood pressure, blood pH, and transport and distribution of numerous endogenous and exogenous ligands. HSA has three structurally homologous domains (domains I, II and III), is almost entirely in the alpha-helical conformation, and is highly stabilized by 17 disulfide bridges. A representative HSA sequence is provided by UniProtKB Primary accession number P02768 and may include other human isoforms thereof.

Anti-HSA AFFIMER® polypeptides comprise an AFFIMER® polypeptide in which at least one of the solvent accessible loops is from the wild-type stefin A protein having amino acid sequences to enable an AFFIMER® polypeptide to bind HSA, selectively, and in some embodiments, with K_{d} of 10⁻⁶M or less.

In some embodiments, the polypeptides bind to HSA with a K_{d} of 1×10⁻⁹ M to 1×10⁻⁶ M at pH 7.4 to 7.6. In some embodiments, the polypeptides bind to HSA with a K_{d} of 1×10⁻⁶ M or less at pH 7.4 to 7.6. In some embodiments, the polypeptides bind to HSA with a K_{d} of 1×10⁻⁷ M or less at pH 7.4 to 7.6. In some embodiments, the polypeptides bind to HSA with a K_{d} of 1×10⁻⁸ M or less at pH 7.4 to 7.6. In some embodiments, the polypeptides bind to HSA with a K_{d} of 1×10⁻⁹ M or less at pH 7.4 to 7.6. In some embodiments, the polypeptides bind to HSA with a K_{d} of 1×10⁻⁹ M to 1×10⁻⁶M at pH 7.4. In some embodiments, the polypeptides bind to HSA with a K_{d} of 1×10⁻⁶ M or less at pH 7.4. In some embodiments, the polypeptides bind to HSA with a K_{d} of 1×10⁻⁷ M or less at pH 7.4. In some embodiments, the polypeptides bind to HSA with a K_{d} of 1×10⁻⁸M or less at pH 7.4. In some embodiments, the polypeptides bind to HSA with a K_{d} of 1×10⁻⁹ M or less at pH 7.4.

In some embodiments, the polypeptides at pH 5.8 to 6.2 bind to HSA with a K_{d} of half a log to 2.5 logs less than the K_{d} for binding to HSA at pH 7.4 to 7.6. In some embodiments, the polypeptides at pH 5.8 to 6.2 bind to HSA with a K_{d} that is at least half a log less than the K_{d} for binding to HSA at pH 7.4 to 7.6. In some embodiments, the polypeptides at pH 5.8 to 6.2 bind to HSA with a K_{d} that is at least one log less than the K_{d} for binding to HSA at pH 7.4 to 7.6. In some embodiments, the polypeptides at pH 5.8 to 6.2 bind to HSA with a K_{d} that is at least 1.5 logs less than the K_{d} for binding to HSA at pH 7.4 to 7.6. In some embodiments, the polypeptides at pH 5.8 to 6.2 bind to HSA with a K_{d} that is at least 2 logs less than the K_{d} for binding to HSA at pH 7.4 to 7.6. In some embodiments, the polypeptides at pH 5.8 to 6.2 bind to HSA with a K_{d} that is at least 2.5 log less than the K_{d} for binding to HSA at pH 7.4 to 7.6. In some embodiments, the polypeptides at pH 6 bind to HSA with a K_{d} of half a log to 2.5 logs less than the K_{d} for binding to HSA at pH 7.4. In some embodiments, the polypeptides at pH 6 bind to HSA with a K_{d} that is at least half a log less than the K_{d} for binding to HSA at pH 7.4. In some embodiments, the polypeptides at pH 6 bind to HSA with a K_{d} that is at least one log less than the K_{d} for binding to HSA at pH 7.4. In some embodiments, the polypeptides at pH 6 bind to HSA with a K_{d} that is at least 1.5 logs less than the K_{d} for binding to HSA at pH 7.4. In some embodiments, the polypeptides at pH 6 bind to HSA with a K_{d} that is at least 2 logs less than the K_{d} for binding to HSA at pH 7.4. In some embodiments, the polypeptides at pH 6 bind to HSA with a K_{d} that is at least 2.5 log less than the K_{d} for binding to HSA at pH 7.4

In some embodiments, the polypeptides have a serum half-life in human patients of greater than 10 hours. In some embodiments, the polypeptides have a serum half-life in human patients of greater than 24 hours. In some embodiments, the polypeptides have a serum half-life in human patients of greater than 48 hours. In some embodiments, the polypeptides have a serum half-life in human patients of greater than 72 hours. In some embodiments, the polypeptides have a serum half-life in human patients of greater than 96 hours. In some embodiments, the polypeptides have a serum half-life in human patients of greater than 120 hours. In some embodiments, the polypeptides have a serum half-life in human patients of greater than 144 hours. In some embodiments, the polypeptides have a serum half-life in human patients of greater than 168 hours. In some embodiments, the polypeptides have a serum half-life in human patients of greater than 192 hours. In some embodiments, the polypeptides have a serum half-life in human patients of greater than 216 hours. In some embodiments, the polypeptides have a serum half-life in human patients of greater than 240 hours. In some embodiments, the polypeptides have a serum half-life in human patients of greater than 264 hours. In some embodiments, the polypeptides have a serum half-life in human patients of greater than 288 hours. In some embodiments, the polypeptides have a serum half-life in human patients of greater than 312 hours. In some embodiments, the polypeptides have a serum half-life in human patients of greater than 336 hours. In some embodiments, the polypeptides have a serum half-life in human patients of greater than 360 hours. In some embodiments, the polypeptides have a serum half-life in human patients of 24 to 360 hours, 48 to 360 hours, 72 to 360 hours, 96 to 360 hours, or 120 to 360 hours.

In some embodiments, the polypeptides have a serum half-life in human patients of greater than 50%, greater than 60%, greater than 70%, or greater than 80% of the serum half-life of HSA. In some embodiments, the polypeptides have a serum half-life in human patients of 50% to 80%, 50% to 90%, or 50% to 100% of the serum half-life of HSA.

In some embodiments, the anti-HSA AFFIMER® polypeptide is derived from the wild-type human stefin A protein having a backbone sequence and in which one or both of loop 2 (designated (Xaa)ₙ) and loop 4 (designated (Xaa)ₘ) are replaced with alternative loop sequences (Xaa)ₙ and (Xaa)ₘ, to have the general formula (I):

FR1-(Xaa)n-FR2-(Xaa)m-FR3 (I),

wherein FR1 is an amino acid sequence having at least 70% identity to MIPGGLSEAK PATPEIQEIV DKVKPQLEEK TNETYGKLEA VQYKTQVLA (SEQ ID NO: 1); FR2 is an amino acid sequence having at least 70% identity to GTNYYIKVRA GDNKYMHLKV FKSL (SEQ ID NO: 2); FR3 is an amino acid sequence having at least 70% identity to EDLVLTGYQV DKNKDDELTG F (SEQ ID NO: 3); Xaa, individually for each occurrence, is an amino acid; and n is an integer from 3 to 20, and m is an integer from 3 to 20.

In some embodiments, FR1 is a polypeptide sequence having 80%-98%, 82%-98%, 84%-98%, 86%-98%, 88%-98%, 90%-98%, 92%-98%, 94%-98%, or 96%-98% homology with SEQ ID NO: 1. In some embodiments, FR1 is a polypeptide sequence having 80%, 82%, 84%, 86%, 88%, 90%, 92%, 94%, 96%, or 95% homology with SEQ ID NO: 1. In some embodiments, FR1 is the polypeptide sequence of SEQ ID NO: 1. In some embodiments, FR2 is a polypeptide sequence having at least 80%-96%, 84%-96%, 88%-96%, or 92%-96% homology with SEQ ID NO: 2. In some embodiments, FR2 is a polypeptide sequence having at least 80%, 84%, 88%, 92%, or 96% homology with SEQ ID NO: 2. In some embodiments, FR2 is a polypeptide sequence having at least 80%, 85%, 90%, 95% or even 98% identity with SEQ ID NO: 2. In some embodiments, FR2 is the polypeptide sequence of SEQ ID NO: 2. In some embodiments, FR3 is a polypeptide sequence having at least 80%-95%, 85%-95%, or 90%-95% homology with SEQ ID No: 3. In some embodiments, FR3 is a polypeptide sequence having at least 80%, 85%, 90%, or 95% homology with SEQ ID NO: 3. In some embodiments, FR3 is the polypeptide sequence of SEQ ID NO: 3.

In some embodiments, an anti-HSA AFFIMER® polypeptide comprises the amino acid sequence represented in general formula (II):

(SEQ ID NO: 166)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF,
(II)

wherein Xaa, individually for each occurrence, is an amino acid; n is an integer from 3 to 20, and m is an integer from 3 to 20; Xaa1 is Gly, Ala, Val, Arg, Lys, Asp, or Glu; Xaa2 is Gly, Ala, Val, Ser or Thr; Xaa3 is Arg, Lys, Asn, Gln, Ser, Thr; Xaa4 is Gly, Ala, Val, Ser or Thr; Xaa5 is Ala, Val, Ile, Leu, Gly or Pro; Xaa6 is Gly, Ala, Val, Asp or Glu; and Xaa7 is Ala, Val, Ile, Leu, Arg or Lys.

In some embodiments, Xaa1 is Gly, Ala, Arg or Lys. In some embodiments, Xaa1 is Gly or Arg. In some embodiments, Xaa2 is Gly, Ala, Val, Ser or Thr. In some embodiments, Xaa2 is Gly or Ser. In some embodiments, Xaa3 is Arg, Lys, Asn, Gln, Ser, Thr. In some embodiments, Xaa3 is Arg, Lys, Asn or Gln. In some embodiments, Xaa3 is Lys or Asn. In some embodiments, Xaa4 is Gly, Ala, Val, Ser or Thr. In some embodiments, Xaa4 is Gly or Ser. In some embodiments, Xaa5 is Ala, Val, Ile, Leu, Gly or Pro. In some embodiments, Xaa5 is Ile, Leu or Pro. In some embodiments, Xaa5 is Leu or Pro. In some embodiments, Xaa6 is Gly, Ala, Val, Asp or Glu. In some embodiments, Xaa6 is Ala, Val, Asp or Glu. In some embodiments, Xaa6 is Ala or Glu. In some embodiments, Xaa7 is Ala, Val, Be, Leu, Arg or Lys. In some embodiments, Xaa7 is Ile, Leu or Arg. In some embodiments, Xaa7 is Leu or Arg.

In some embodiments, an anti-HSA AFFIMER® polypeptide comprises the amino acid sequence represented in general formula (III):

(SEQ ID NO: 167)
MIPRGLSEAKPATPEIQEIVDKVKPQLEEKTNETYGKLEAVQYKT
QVLA-(Xaa)n-STNYYIKVRAGDNKYMHLKVFNGP-(Xaa)m-ADR
VLTGYQVDKNKDDELTGF,
(III)

wherein Xaa, individually for each occurrence, is an amino acid; n is an integer from 3 to 20, and m is an integer from 3 to 20. In some embodiments, n is 3, 4, 5, 6, 7, 8, 9, 10, 11, 12, 13, 14, 15, 16, 17, 18, 19, or 20. In some embodiments, n is 8 to 10, 7 to 11, 6 to 12, 5 to 13, 4 to 14, or 3 to 15. In some embodiments, m is 3, 4, 5, 6, 7, 8, 9, 10, 11, 12, 13, 14, 15, 16, 17, 18, 19, or 20. In some embodiments, m is 8 to 10, 7 to 11, 6 to 12, 5 to 13, 4 to 14, or 3 to 15.

In some embodiments, (Xaa)ₙ is represented by formula (IV):

aa1-aa2-aa3-aa4-aa5-aa6-aa7-aa8-aa9 (IV),

wherein aa1 is an amino acid with a neutral polar hydrophilic side chain; aa2 is an amino acid with a neutral nonpolar hydrophobic side chain; aa3 is an amino acid with a neutral nonpolar hydrophobic side chain; aa4 is an amino acid with a neutral polar hydrophilic side chain; aa5 is an amino acid with a positively charged polar hydrophilic side chain; aa6 is an amino acid with a positively charged polar hydrophilic side chain; aa7 is an amino acid with a neutral nonpolar hydrophobic side chain; aa8 is an amino acid with a neutral nonpolar hydrophobic side chain; and aa9 is an amino acid with a neutral nonpolar hydrophilic side chain.

In some embodiments, (Xaa)ₘ is represented by formula (V):

aa1-aa2-aa3-aa4-aa5-aa6-aa7-aa8-aa9 (V),

wherein aa1 is an amino acid with a neutral nonpolar hydrophobic side chain; aa2 is an amino acid with a positively charged polar hydrophilic side chain; aa3 is an amino acid with a neutral nonpolar hydrophobic side chain; aa4 is an amino acid with a positively charged polar hydrophilic side chain; aa5 is an amino acid with a neutral polar hydrophilic side chain; aa6 is an amino acid with a neutral polar hydrophilic side chain; aa7 is an amino acid with a negatively charged polar hydrophilic side chain; aa8 is an amino acid with a positively charged polar hydrophilic side chain; and aa9 is an amino acid with a neutral nonpolar hydrophilic side chain.

Examples of amino acids with a neutral nonpolar hydrophilic side chain include cysteine (Cys) and glycine (Gly). In some embodiments, the amino acid with a neutral nonpolar hydrophilic side chain is Cys. In some embodiments, the amino acid with a neutral nonpolar hydrophilic side chain is Gly.

Examples of amino acids with a neutral nonpolar hydrophobic side chain include alanine (Ala), isoleucine (Ile), leucine (Leu), methionine (Met), phenylalanine (Phe), proline (Pro), tryptophan (Trp), and valine (Val). In some embodiments, the amino acid with a neutral nonpolar hydrophobic side chain is Ala. In some embodiments, the amino acid with a neutral nonpolar hydrophobic side chain is Ile. In some embodiments, the amino acid with a neutral nonpolar hydrophobic side chain is Leu. In some embodiments, the amino acid with a neutral nonpolar hydrophobic side chain is Met. In some embodiments, the amino acid with a neutral nonpolar hydrophobic side chain is Phe. In some embodiments, the amino acid with a neutral nonpolar hydrophobic side chain is Pro. In some embodiments, the amino acid with a neutral nonpolar hydrophobic side chain is Trp. In some embodiments, the amino acid with a neutral nonpolar hydrophobic side chain is Val.

Examples of amino acids with a neutral polar hydrophilic side chain include asparagine (Asn), glutamine (Gln), serine (Ser), threonine (Thr), and tyrosine (Tyr). In some embodiments, the amino acid with a neutral polar hydrophilic side chain is Asn. In some embodiments, the amino acid with a neutral polar hydrophilic side chain is Gln. In some embodiments, the amino acid with a neutral polar hydrophilic side chain is Ser. In some embodiments, the amino acid with a neutral polar hydrophilic side chain is Thr. In some embodiments, the amino acid with a neutral polar hydrophilic side chain is Tyr.

Examples of amino acids with a positively charged polar hydrophilic side chain include arginine (Arg), histidine (His), and lysine (Lys). In some embodiments, the amino acid with a positively charged polar hydrophilic side is Arg. In some embodiments, the amino acid with a positively charged polar hydrophilic side is His. In some embodiments, the amino acid with a positively charged polar hydrophilic side is Lys.

Examples of amino acids with a negatively charged polar hydrophilic side chain include aspartate (Asp) and glutamate (Glu). In some embodiments, the amino acid with a negatively charged polar hydrophilic side chain is Asp. In some embodiments, the amino acid with a negatively charged polar hydrophilic side chain is Glu.

In some embodiments, (Xaa)ₙ is represented by formula (IV):

(SEQ ID NO: 180)
aa1-aa2-aa3-aa4-aa5-aa6-aa7-aa8-aa9,
(IV)

wherein aa1 is an amino acid selected from Asp, Gly, Asn, and Va1; aa2 is an amino acid selected from Trp, Tyr, His, and Phe; aa3 is an amino acid selected from Trp, Tyr, Gly, Trp, and Phe; aa4 is an amino acid selected from Gln, Ala, and Pro; aa5 is an amino acid selected from Ala, Gln, Glu, Arg, and Ser; aa6 is an amino acid selected from Lys, Arg, and Tyr; aa7 is an amino acid selected from Trp and Gln; aa8 is an amino acid selected from Pro and His; and/or aa9 is an amino acid selected from His, Gly, and Gln. In some embodiments, aa1 is Asp. In some embodiments, aa1 is Gly. In some embodiments, aa1 is Asn. In some embodiments, aa2 is Trp. In some embodiments, aa2 is Tyr. In some embodiments, aa2 is His. In some embodiments, aa2 is Phe. In some embodiments, aa3 is Trp. In some embodiments, aa3 is Tyr. In some embodiments, aa3 is Gly. In some embodiments, aa3 is Trp. In some embodiments, aa3 is Phe. In some embodiments, aa4 is Gln. In some embodiments, aa4 is Ala. In some embodiments, aa4 is Pro. In some embodiments, aa5 is Ala. In some embodiments, aa5 is Gln. In some embodiments, aa5 is Glu. In some embodiments, aa5 is Arg. In some embodiments, aa5 is Ser. In some embodiments, aa6 is Lys. In some embodiments, aa6 is Arg. In some embodiments, aa6 is Tyr. In some embodiments, aa7 is Trp. In some embodiments, aa7 is Gln. In some embodiments, aa8 is Pro. In some embodiments, aa8 is His. In some embodiments, aa9 is His. In some embodiments, aa9 is Gly. In some embodiments, aa9 is Gln.

In some embodiments, (Xaa)ₘ is represented by formula (IV):

(SEQ ID NO: 181)
aa1-aa2-aa3-aa4-aa5-aa6-aa7-aa8-aa9,
(IV)

wherein aa1 is an amino acid selected from Tyr, Phe, Trp, and Asn; aa2 is an amino acid selected from Lys, Pro, His, Ala, and Thr; aa3 is an amino acid selected from Val, Asn, Gly, Gln, Ala, and Phe; aa4 is an amino acid selected from His, Thr, Lys, Trp, Lys, Val, and Arg; aa5 is an amino acid selected from Gln, Ser, Gly, Pro, and Asn; aa6 is an amino acid selected from Ser, Tyr, Glu, Leu, Lys, and Thr; aa7 is an amino acid selected from Ser, Asp, Val, and Lys; aa8 is an amino acid selected from Gly, Leu, Ser, Pro, His, Asp, and Arg; and/or aa9 is an amino acid selected from Gly, Gln, Glu, and Ala. In some embodiments, aa1 is Tyr. In some embodiments, aa1 is Phe. In some embodiments, aa1 is Trp. In some embodiments, aa1 is Asn. In some embodiments, aa2 is Lys. In some embodiments, aa2 is Pro. In some embodiments, aa2 is His. In some embodiments, aa2 is Ala. In some embodiments, aa2 is Thr. In some embodiments, aa3 is Val. In some embodiments, aa3 is Asn. In some embodiments, aa3 is Gly. In some embodiments, aa3 is Gln. In some embodiments, aa3 is Ala. In some embodiments, aa3 is Phe. In some embodiments, aa4 is His. In some embodiments, aa4 is Thr. In some embodiments, aa4 is Lys. In some embodiments, aa4 is Trp. In some embodiments, aa4 is Lys. In some embodiments, aa4 is Val. In some embodiments, aa4 is Arg. In some embodiments, aa5 is Gln. In some embodiments, aa5 is Ser. In some embodiments, aa5 is Gly. In some embodiments, aa5 is Pro. In some embodiments, aa5 is Asn. In some embodiments, aa6 is Ser. In some embodiments, aa6 is Tyr. In some embodiments, aa6 is Glu. In some embodiments, aa6 is Leu. In some embodiments, aa6 is Lys. In some embodiments, aa6 is Thr. In some embodiments, aa7 is Ser. In some embodiments, aa7 is Asp. In some embodiments, aa7 is Val. In some embodiments, aa7 is Lys. In some embodiments, aa8 is Gly. In some embodiments, aa8 is Leu. In some embodiments, aa8 is Ser. In some embodiments, aa8 is Pro. In some embodiments, aa8 is His. In some embodiments, aa8 is Asp. In some embodiments, aa8 is Arg. In some embodiments, aa9 is Gly. In some embodiments, aa9 is Gln. In some embodiments, aa9 is Glu. In some embodiments, aa9 is Ala. In some embodiments, (Xaa)ₙ is represented by formula (V):

(SEQ ID NO: 179)
Asn-aa1-aa2-Gln-Gln-Arg-Arg-Trp-Pro-Gly,
(V)

wherein aa1 is an amino acid selected from Trp and Phe; and aa2 is an amino acid selected from Tyr and Phe. In some embodiments, aa1 is Trp. In some embodiments, aa1 is Phe. In some embodiments, aa2 is Tyr. In some embodiments, aa2 it Phe.

In some embodiments, (Xaa)ₙ is represented by formula (VI):

aa1-aa2-Trp-aa3-aa4-Lys-Trp-Pro-aa5 (VI),

wherein aa1 is an amino acid selected from Asp and Gly; aa2 is an amino acid selected from Trp, Tyr, and Phe; aa3 is an amino acid selected from Gln and Ala; aa4 is an amino acid selected from Ala and Ser; and aa5 is an amino acid selected from His and Gly. In some embodiments, aa1 is Asp. In some embodiments, aa1 is Gly. In some embodiments, aa2 is Trp. In some embodiments, aa2 is Tyr. In some embodiments, aa2 is Phe. In some embodiments, aa3 is Gln. In some embodiments, aa3 is Ala. In some embodiments, aa4 is Ala. In some embodiments, aa4 is Ser. In some embodiments, aa5 is His. In some embodiments, aa5 is Gly.

In some embodiments, (Xaa)ₙ is represented by formula (VII):

aa1-aa2-aa3-aa4-aa5-aa6-Trp-Pro-Gly (VII),

wherein aa1 is an amino acid selected from Gly and Asn; aa2 is an amino acid selected from Tyr, Phe, Trp, and His; aa3 is an amino acid selected from Trp, Tyr, and Phe; aa4 is an amino acid selected from Ala and Gln; aa5 is an amino acid selected from Ala, Ser, Gln, and Arg; and aa6 is an amino acid selected from Lys, Arg, and Tyr. In some embodiments, aa1 is Gly. In some embodiments, aa1 is Asn. In some embodiments, aa2 is Tyr. In some embodiments, aa2 is Phe. In some embodiments, aa2 is Trp. In some embodiments, aa2 is His. In some embodiments, aa3 is Trp. In some embodiments, aa3 is Tyr. In some embodiments, aa3 is Phe. In some embodiments, aa4 is Ala. In some embodiments, aa4 is Gln. In some embodiments, aa5 is Ala. In some embodiments, aa5 is Ser. In some embodiments, aa5 is Gln. In some embodiments, aa5 is Arg. In some embodiments, aa6 is Lys. In some embodiments, aa6 is Arg. In some embodiments, aa6 is Tyr.

In some embodiments, (Xaa)ₙ is represented by formula (IX):

Gly-aa1-aa2-Ala-aa3-aa4-Trp-Pro-Gly (IX),

wherein aa1 is an amino acid selected from Tyr, Phe, and His; aa2 is an amino acid selected from Trp and Tyr; aa3 is an amino acid selected from Ala, Ser, and Arg; and aa4 is an amino acid selected from Lys and Tyr. In some embodiments, aa1 is Tyr. In some embodiments, aa1 is Phe His. In some embodiments, aa1 is His. In some embodiments, aa2 is Trp. In some embodiments, aa2 is Tyr. In some embodiments, aa3 is Ala. In some embodiments, aa3 is Ser. In some embodiments, aa3 is Arg. In some embodiments, aa4 is Lys. In some embodiments, aa4 is Tyr.

In some embodiments, (Xaa)ₙ is represented by formula (X):

aa1-aa2-aa3-Gln-aa4-aa5-Trp-Pro-aa6 (X),

wherein aa1 is an amino acid selected from Asp and Asn; aa2 is an amino acid selected from Trp and Phe; aa3 is an amino acid selected from Trp, Tyr, and Phe; aa4 is an amino acid selected from Ala, Gln, and Arg; aa5 is an amino acid selected from Lys and Arg; and aa6 is an amino acid selected from His and Gly. In some embodiments, aa1 is Asp. In some embodiments, aa1 is Asn. In some embodiments, aa2 is Trp. In some embodiments, aa2 is Phe. In some embodiments, aa3 is Trp. In some embodiments, aa3 is Tyr. In some embodiments, aa3 is Phe. In some embodiments, aa4 is Ala. In some embodiments, aa4 is Gln. In some embodiments, aa4 is Arg. In some embodiments, aa5 is Lys. In some embodiments, aa5 is Arg. In some embodiments, aa6 is His. In some embodiments, aa6 is Gly.

In some embodiments, an anti-HSA AFFIMER® polypeptide comprises a loop 2 amino acid sequence selected from any one of SEQ ID NOS: 4-56 (Table 1). In some embodiments, an anti-HSA AFFIMER® polypeptide comprises a loop 4 amino acid sequence selected from any one of SEQ ID NOS: 57-109 (Table 1).

TABLE 1
Examples of HSA AFFIMER ® Loop Sequences

| | | SEQ | | SEQ |
|---|---|---|---|---|
| Name | Loop 2 | ID NO: | Loop 4 | ID NO: |
| HSA-00 | WTQPKNEHH | 4 | RFKYFAHYQ | 57 |
| HSA-01 | HLKHTDAQP | 5 | FHDFWHRRW | 58 |
| HSA-02 | HDQDVLHAW | 6 | DWYHYWWEV | 59 |
| HSA-03 | KFHRQEWAD | 7 | STRSIHVTT | 60 |
| HSA-04 | PEDFWDPEH | 8 | KQHHHYLDK | 61 |
| HSA-05 | VVRTTGHVV | 9 | HSAQDREIP | 62 |
| HSA-06 | YWWFCTGQS | 10 | WVQSGYNSQ | 63 |
| HSA-07 | IHHRQARSL | 11 | AVFWGKWSD | 64 |
| HSA-08 | SHRRRAYIW | 12 | QSFDKPWTT | 65 |
| HSA-09 | WDSHHWRAP | 13 | HYPLKYSFE | 66 |
| HSA-10 | DKRVKYGQ | 14 | WHHPWHRNR | 67 |
| HSA-11 | SDWVYALQL | 15 | DPWWAWVVW | 68 |
| HSA-12 | FWWFWY | 16 | FDNQDLIQY | 69 |
| HSA-13 | VRDWPWNTF | 17 | EKKNWYKWD | 70 |
| HSA-14 | QKKRDEDYI | 18 | DRHKSRWGI | 71 |
| HSA-15 | GVHEEPRKL | 19 | LNPFTPSVT | 72 |
| HSA-16 | EWWQKHWPS | 20 | YKGALLNHD | 73 |
| HSA-17 | NFFQRRWPG | 21 | WKFRNTERG | 74 |
| HSA-18 | DWWQAKWPH | 22 | YKVHQSSGG | 75 |
| HSA-19 | GIWQSRWPG | 23 | FHPIAGRPW | 76 |
| HSA-20 | GYWAAKWPG | 24 | FPNTSYDLQ | 77 |
| HSA-21 | GFYADHWPG | 25 | FAHYNLKSG | 78 |
| HSA-22 | NWYQQRWPG | 26 | WHNYGESSG | 79 |
| HSA-23 | GFYARHWPG | 27 | KFYYADHQW | 80 |
| HSA-24 | DFWKAHWPG | 28 | YTHADPHSQ | 81 |
| HSA-25 | DFYSVRWPG | 29 | FGVPQLGAG | 82 |
| HSA-26 | YWAANHASK | 30 | YSGFPFAGF | 83 |
| HSA-27 | IKRLEHWEY | 31 | WFSWPYTPL | 84 |
| HSA-28 | EWDSPWSEN | 32 | YYHPSIQST | 85 |
| HSA-29 | KHKNLRWPF | 33 | FLGWKDTVV | 86 |
| HSA-30 | RHFPKQTNW | 34 | DWWKWWWAK | 87 |
| HSA-31 | VWGPEYQHQ | 35 | NAGWPLVPE | 88 |
| HSA-32 | TWKNNGQDV | 36 | YALDPFGGK | 89 |
| HSA-33 | ATWLNYYLP | 37 | GYKFWGVSD | 90 |
| HSA-34 | DQESLFLNN | 38 | QGKQYILLR | 91 |
| HSA-35 | GFYAQHWPD | 39 | YKRHSAHDY | 92 |
| HSA-36 | GHYARYWPG | 40 | WAQKSKVHQ | 93 |
| HSA-37 | GFWASKWPG | 41 | FTAVSKKDA | 94 |
| HSA-38 | GFWQRKWPN | 42 | WGDKENIWF | 95 |
| HSA-39 | VWPADNDLK | 43 | WSGHPWVQK | 96 |
| HSA-40 | HWAWTSPGY | 44 | YADYPLSPK | 97 |
| HSA-41 | NFFQRRWPG | 45 | WKFRNTDRG | 98 |
| HSA-42 | HHSHRLKGQ | 46 | QTVATHYHY | 99 |
| HSA-43 | YQNTIFLSI | 47 | WHAKHLLSH | 100 |
| HSA-44 | FQDQFTWSQ | 48 | SGIKKADSV | 101 |
| HSA-45 | GEPHWPWQA | 49 | KANLINVKS | 102 |
| HSA-46 | ADPRHPWVE | 50 | WKSHVEVRS | 103 |
| HSA-47 | FHKRFQSQG | 51 | WVTQKYIIQ | 104 |
| HSA-48 | EWWQNRWPN | 52 | WEHAKDWPT | 105 |
| HSA-49 | EWYQTRWPG | 53 | FHSKVLDKA | 106 |
| HSA-50 | EFWQRHWPG | 54 | YGAQKQAVW | 107 |
| HSA-51 | KFYERHWPG | 55 | FSASHFTSQ | 108 |
| Consensus | GWWQRRWPG | 56 | X₁X₂AX₃KX₄DX₅Q | 109 |

In some embodiments, (Xaa)ₙ comprises an amino acid sequence having at least 80% or at least 90% identity to the amino acid sequence of any one of SEQ ID NOS: 4-55. In some embodiments, (Xaa)ₙ comprises an amino acid sequence having 80% to 90% identity to the amino acid sequence of any one of SEQ ID NOS: 4-55. In some embodiments, (Xaa)ₙ comprises the amino acid sequence of any one of SEQ ID NOS: 4-55.

In some embodiments, (Xaa)ₙ comprises an amino acid sequence having at least 80% or at least 90% identity to the amino acid sequence of any one of SEQ ID NOS: 22, 24, 26, 35, 40, 41, and 45. In some embodiments, (Xaa)ₙ comprises an amino acid sequence having at least 80% identity to the amino acid sequence of SEQ ID NO: 22. In some embodiments, (Xaa)ₙ comprises an amino acid sequence having at least 80% identity to the amino acid sequence of SEQ ID NO: 24. In some embodiments, (Xaa)ₙ comprises an amino acid sequence having at least 80% identity to the amino acid sequence of SEQ ID NO: 26. In some embodiments, (Xaa)ₙ comprises an amino acid sequence having at least 80% identity to the amino acid sequence of SEQ ID NO: 35. In some embodiments, (Xaa)ₙ comprises an amino acid sequence having at least 80% identity to the amino acid sequence of SEQ ID NO: 40. In some embodiments, (Xaa)ₙ comprises an amino acid sequence having at least 80% identity to the amino acid sequence of SEQ ID NO: 41. In some embodiments, (Xaa)ₙ comprises an amino acid sequence having at least 80% identity to the amino acid sequence of SEQ ID NO: 45. In some embodiments, (Xaa)ₙ comprises an amino acid sequence having at least 90% identity to the amino acid sequence of SEQ ID NO: 22. In some embodiments, (Xaa)ₙ comprises an amino acid sequence having at least 90% identity to the amino acid sequence of SEQ ID NO: 24. In some embodiments, (Xaa)ₙ comprises an amino acid sequence having at least 90% identity to the amino acid sequence of SEQ ID NO: 26. In some embodiments, (Xaa)ₙ comprises an amino acid sequence having at least 90% identity to the amino acid sequence of SEQ ID NO: 35. In some embodiments, (Xaa)ₙ comprises an amino acid sequence having at least 90% identity to the amino acid sequence of SEQ ID NO: 40. In some embodiments, (Xaa)ₙ comprises an amino acid sequence having at least 90% identity to the amino acid sequence of SEQ ID NO: 41. In some embodiments, (Xaa)ₙ comprises an amino acid sequence having at least 90% identity to the amino acid sequence of SEQ ID NO: 45.

In some embodiments, (Xaa)ₙ comprises an amino acid sequence having 80% to 90% identity to the amino acid sequence of any one of SEQ ID NOS: 22, 24, 26, 35, 40, 41, and 45. In some embodiments, (Xaa)ₙ comprises an amino acid sequence having 80% to 90% identity to the amino acid sequence of SEQ ID NO: 22. In some embodiments, (Xaa)ₙ comprises an amino acid sequence having 80% to 90% identity to the amino acid sequence of SEQ ID NO: 24. In some embodiments, (Xaa)ₙ comprises an amino acid sequence having 80% to 90% identity to the amino acid sequence of SEQ ID NO: 26. In some embodiments, (Xaa)ₙ comprises an amino acid sequence having 80% to 90% identity to the amino acid sequence of SEQ ID NO: 35. In some embodiments, (Xaa)ₙ comprises an amino acid sequence having 80% to 90% identity to the amino acid sequence of SEQ ID NO: 40. In some embodiments, (Xaa)ₙ comprises an amino acid sequence having 80% to 90% identity to the amino acid sequence of SEQ ID NO: 41. In some embodiments, (Xaa)ₙ comprises an amino acid sequence having 80% to 90% identity to the amino acid sequence of SEQ ID NO: 45.

In some embodiments, (Xaa)ₙ comprises the amino acid sequence of any one of SEQ ID NOS: 22, 24, 26, 35, 40, 41, and 45. In some embodiments, (Xaa)ₙ comprises the amino acid sequence of SEQ ID NO: 22. In some embodiments, (Xaa)ₙ comprises the amino acid sequence of SEQ ID NO: 24. In some embodiments, (Xaa)ₙ comprises the amino acid sequence of SEQ ID NO: 26. In some embodiments, (Xaa)ₙ comprises the amino acid sequence of SEQ ID NO: 35. In some embodiments, (Xaa)ₙ comprises the amino acid sequence of SEQ ID NO: 40. In some embodiments, (Xaa)ₙ comprises the amino acid sequence of SEQ ID NO: 41. In some embodiments, (Xaa)ₙ comprises the amino acid sequence of SEQ ID NO: 45.

In some embodiments, (Xaa)ₘ comprises an amino acid sequence having at least 80% or at least 90% identity to the amino acid sequence of any one of SEQ ID NOS: 57-108. In some embodiments, (Xaa)ₘ comprises an amino acid sequence having 80% to 90% identity to the amino acid sequence of any one of SEQ ID NOS: 57-108. In some embodiments, (Xaa)ₘ comprises the amino acid sequence of any one of SEQ ID NOS: 57-108.

In some embodiments, (Xaa)ₘ comprises an amino acid sequence having at least 80% or at least 90% identity to the amino acid sequence of any one of SEQ ID NOS: 75, 77, 79, 88, 93, 94, and 98. In some embodiments, (Xaa)ₘ comprises an amino acid sequence having at least 80% identity to the amino acid sequence of SEQ ID NO: 75. In some embodiments, (Xaa)ₘ comprises an amino acid sequence having at least 80% identity to the amino acid sequence of SEQ ID NO: 77. In some embodiments, (Xaa)ₘ comprises an amino acid sequence having at least 80% identity to the amino acid sequence of SEQ ID NO: 79. In some embodiments, (Xaa)ₘ comprises an amino acid sequence having at least 80% identity to the amino acid sequence of SEQ ID NO: 88. In some embodiments, (Xaa)ₘ comprises an amino acid sequence having at least 80% identity to the amino acid sequence of SEQ ID NO: 93. In some embodiments, (Xaa)ₘ comprises an amino acid sequence having at least 80% identity to the amino acid sequence of SEQ ID NO: 94. In some embodiments, (Xaa)ₘ comprises an amino acid sequence having at least 80% identity to the amino acid sequence of SEQ ID NO: 98. In some embodiments, (Xaa)ₘ comprises an amino acid sequence having at least 90% identity to the amino acid sequence of SEQ ID NO: 75. In some embodiments, (Xaa)ₘ comprises an amino acid sequence having at least 90% identity to the amino acid sequence of SEQ ID NO: 77. In some embodiments, (Xaa)ₘ comprises an amino acid sequence having at least 90% identity to the amino acid sequence of SEQ ID NO: 79. In some embodiments, (Xaa)ₘ comprises an amino acid sequence having at least 90% identity to the amino acid sequence of SEQ ID NO: 88. In some embodiments, (Xaa)ₘ comprises an amino acid sequence having at least 90% identity to the amino acid sequence of SEQ ID NO: 93. In some embodiments, (Xaa)ₘ comprises an amino acid sequence having at least 90% identity to the amino acid sequence of SEQ ID NO: 94. In some embodiments, (Xaa)ₘ comprises an amino acid sequence having at least 90% identity to the amino acid sequence of SEQ ID NO: 98.

In some embodiments, (Xaa)ₘ comprises an amino acid sequence having 80% to 90% identity to the amino acid sequence of any one of SEQ ID NOS: 75, 77, 79, 88, 93, 94, and 98. In some embodiments, (Xaa)ₘ comprises the amino acid sequence of any one of SEQ ID NOS: 75, 77, 79, 88, 93, 94, and 98. In some embodiments, (Xaa)ₘ comprises an amino acid sequence having 80% to 90% identity to the amino acid sequence of SEQ ID NO: 75. In some embodiments, (Xaa)ₘ comprises an amino acid sequence having 80% to 90% identity to the amino acid sequence of SEQ ID NO: 77. In some embodiments, (Xaa)ₘ comprises an amino acid sequence having 80% to 90% identity to the amino acid sequence of SEQ ID NO: 79. In some embodiments, (Xaa)ₘ comprises an amino acid sequence having 80% to 90% identity to the amino acid sequence of SEQ ID NO: 88. In some embodiments, (Xaa)ₘ comprises an amino acid sequence having 80% to 90% identity to the amino acid sequence of SEQ ID NO: 93. In some embodiments, (Xaa)ₘ comprises an amino acid sequence having 80% to 90% identity to the amino acid sequence of SEQ ID NO: 94. In some embodiments, (Xaa)ₘ comprises an amino acid sequence having 80% to 90% identity to the amino acid sequence of SEQ ID NO: 98.

In some embodiments, an anti-HSA AFFIMER® polypeptide comprises an amino acid sequence selected from any one of SEQ ID NOS: 110-116, and 138 (Table 2).

TABLE 2
Examples of HSA AFFIMER ® Polypeptide Sequences

| Name | Sequence | SEQ ID NO: |
|---|---|---|
| HSA-18 | MIPRGLSEAKPATPEIQEIVDKVKPQLEEKTNETYGKLEAVQYKTQVLA | 110 |
| | [symbol] STNYYIKVRAGDNKYMHLKVFNGP [symbol] AD | |
| | RVLTGYQVDKNKDDELTGF | |
| HSA-20 | MIPRGLSEAKPATPEIQEIVDKVKPQLEEKTNETYGKLEAVQYKTQVLA | 111 |
| | [symbol] STNYYIKVRAGDNKYMHLKVFNGP [symbol] ADR | |
| | VLTGYQVDKNKDDELTGF | |
| HSA-22 | MIPRGLSEAKPATPEIQEIVDKVKPQLEEKTNETYGKLEAVQYKTQVLA | 112 |
| | [symbol] STNYYIKVRADNKYMHLKVFNGP [symbol] ADR | |
| | VLTGYQVDKNKDDELTGF | |
| HSA-31 | MIPRGLSEAKPATPEIQEIVDKVKPQLEEKTNETYGKLEAVQYKTQVLA | 113 |
| | [symbol] STNYYIKVRAGDNKYMHLKVFNGP [symbol] ADR | |
| | VLTGYQVDKNKDDELTGF | |
| HSA-36 | MIPRGLSEAKPATPEIQEIVDKVKPQLEEKTNETYGKLEAVQYKTQVLA | 114 |
| | [symbol] STNYYIKVRAGDNKYMHLKVFNGP [symbol] ADR | |
| | VLTGYQVDKNKDDELTGF | |
| HSA-37 | MIPRGLSEAKPATPEIQEIVDKVKPQLEEKTNETYGKLEAVQYKTQVLA | 115 |
| | [symbol] STNYYIKVRAGDNKYMHLKVFNGP [symbol] ADR | |
| | VLTGYQVDKNKDDELTGF | |
| HSA-41 | MIPRGLSEAKPATPEIQEIVDKVKPQLEEKTNETYGKLEAVQYKTQVLA | 116 |
| | [symbol] STNYYIKVRAGDNKYMHLKVFNGP [symbol] ADR | |
| | VLTGYQVDKNKDDELTGF | |
| HSA-41 | MIPRGLSEAKPATPEIQEIVDKVKPQLEEKTNETYGKLEAVQYKTQVLA | 138 |
| CQ | [symbol] STNYYIKVRAGDNKYMHLKVFNGP [symbol] ADR | |
| | VLTGYQVDKNKDDELTGFAAAGGRAEQKLISEEDLGCAENLYFQGGA | |
| | AGHHHHHH | |
| HSA-41 x9 | MIPRGLSEAKPATPEIQEIVDKVKPQLEEKTNETYGKLEAVQYKTQVL | 141 |
| glycine | ANFFQRRWPGSTNYYIKVRAGDNKYMHLKVFNGPGGGGGGGGGADRVL | |
| loops | TGYQVDKNKDDELTGF | |
| HSA-41 | MIPRGLSEAKPATPEIQEIVDKVKPQLEEKTNETYGKLEAVQYKTQVL | 142 |
| loop 4 | ANFFQRRWPGSTNYYIKVRAGDNKYMHLKVFNGPADRVLTGYQVDKNK | |
| deletion | DDELTGF | |
| HSA-41 | MIPRGLSEAKPATPEIQEIVDKVKPQLEEKTNETYGKLEAVQYKTQVL | 143 |
| N50A | AAFFQRRWPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 144 |
| F51A | ANAFQRRWPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 145 |
| F52A | ANFAQRRWPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 146 |
| Q53A | ANFFARRWPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 147 |
| R54A | ANFFQARWPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 148 |
| R55A | ANFFQRAWPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 149 |
| W56A | ANFFQRRAPGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 150 |
| P57A | ANFFQRRWAGSTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 151 |
| G58A | ANFFQRRWPASTNYYIKVRAGDNKYMHLKVFNGPWKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 152 |
| W83A | ANFFQRRWPGSTNYYIKVRAGDNKYMHLKVFNGPAKFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 153 |
| K84A | ANFFQRRWPGSTNYYIKVRAGDNKYMHLKVFNGPWAFRNTDRGADRVL | |
| | TGYQVDKNKDDELTGF | |
| HSA-41 | MIPRGLSEAKPATPEIQEIVDKVKPQLEEKTNETYGKLEAVQYKTQVL | 154 |
| F85A | ANFFQRRWPGSTNYYIKVRAGDNKYMHLKVFNGPWKARNTDRGADRVL | |
| | TGYQVDKNKDDELTGF | |
| HSA-41 | MIPRGLSEAKPATPEIQEIVDKVKPQLEEKTNETYGKLEAVQYKTQVL | 155 |
| R86A | ANFFQRRWPGSTNYYIKVRAGDNKYMHLKVFNGPWKFANTDRGADRVL | |
| | TGYQVDKNKDDELTGF | |
| HSA-41 | MIPRGLSEAKPATPEIQEIVDKVKPQLEEKTNETYGKLEAVQYKTQVL | 156 |
| N87A | ANFFQRRWPGSTNYYIKVRAGDNKYMHLKVFNGPWKFRATDRGADRVL | |
| | TGYQVDKNKDDELTGF | |
| HSA-41 | MIPRGLSEAKPATPEIQEIVDKVKPQLEEKTNETYGKLEAVQYKTQVL | 157 |
| T88A | ANFFQRRWPGSTNYYIKVRAGDNKYMHLKVFNGPWKFRNADRGADRVL | |
| | TGYQVDKNKDDELTGF | |
| HSA-41 | MIPRGLSEAKPATPEIQEIVDKVKPQLEEKTNETYGKLEAVQYKTQVL | 158 |
| D89A | ANFFQRRWPGSTNYYIKVRAGDNKYMHLKVFNGPWKFRNTARGADRVL | |
| | TGYQVDKNKDDELTGF | |
| HSA-41 | MIPRGLSEAKPATPEIQEIVDKVKPQLEEKTNETYGKLEAVQYKTQVL | 159 |
| R90A | ANFFQRRWPGSTNYYIKVRAGDNKYMHLKVFNGPWKFRNTDAGADRVL | |
| | TGYQVDKNKDDELTGF | |
| HSA-41 | MIPRGLSEAKPATPEIQEIVDKVKPQLEEKTNETYGKLEAVQYKTQVL | 160 |
| G91A | ANFFQRRWPGSTNYYIKVRAGDNKYMHLKVFNGPWKFRNTDRAADRVL | |
| | TGYQVDKNKDDELTGF | |

In some embodiments, an anti-HSA AFFIMER® polypeptide comprises an amino acid sequence having at least 80% or at least 90% identity to the amino acid sequence of any one of SEQ ID NOS: 110-116 and 138. In some embodiments, an anti-HSA AFFIMER® polypeptide comprises an amino acid sequence having at least 80% identity to the amino acid sequence of SEQ ID NO: 110. In some embodiments, an anti-HSA AFFIMER® polypeptide comprises an amino acid sequence having at least 80% identity to the amino acid sequence of SEQ ID NO: 111. In some embodiments, an anti-HSA AFFIMER® polypeptide comprises an amino acid sequence having at least 80% identity to the amino acid sequence of SEQ ID NO: 112. In some embodiments, an anti-HSA AFFIMER® polypeptide comprises an amino acid sequence having at least 80% identity to the amino acid sequence of SEQ ID NO: 113. In some embodiments, an anti-HSA AFFIMER® polypeptide comprises an amino acid sequence having at least 80% identity to the amino acid sequence of SEQ ID NO: 114. In some embodiments, an anti-HSA AFFIMER® polypeptide comprises an amino acid sequence having at least 80% identity to the amino acid sequence of SEQ ID NO: 115. In some embodiments, an anti-HSA AFFIMER® polypeptide comprises an amino acid sequence having at least 80% identity to the amino acid sequence of SEQ ID NO: 116. In some embodiments, an anti-HSA AFFIMER® polypeptide comprises an amino acid sequence having at least 80% identity to the amino acid sequence of SEQ ID NO: 138. In some embodiments, an anti-HSA AFFIMER® polypeptide comprises an amino acid sequence having at least 90% identity to the amino acid sequence of SEQ ID NO: 110. In some embodiments, an anti-HSA AFFIMER® polypeptide comprises an amino acid sequence having at least 90% identity to the amino acid sequence of SEQ ID NO: 111. In some embodiments, an anti-HSA AFFIMER® polypeptide comprises an amino acid sequence having at least 90% identity to the amino acid sequence of SEQ ID NO: 112. In some embodiments, an anti-HSA AFFIMER® polypeptide comprises an amino acid sequence having at least 90% identity to the amino acid sequence of SEQ ID NO: 113. In some embodiments, an anti-HSA AFFIMER® polypeptide comprises an amino acid sequence having at least 90% identity to the amino acid sequence of SEQ ID NO: 114. In some embodiments, an anti-HSA AFFIMER® polypeptide comprises an amino acid sequence having at least 90% identity to the amino acid sequence of SEQ ID NO: 115. In some embodiments, an anti-HSA AFFIMER® polypeptide comprises an amino acid sequence having at least 90% identity to the amino acid sequence of SEQ ID NO: 116. In some embodiments, an anti-HSA AFFIMER® polypeptide comprises an amino acid sequence having at least 90% identity to the amino acid sequence of SEQ ID NO: 138.

In some embodiments, an anti-HSA AFFIMER® polypeptide comprises an amino acid sequence having 80% to 90% identity to the amino acid sequence of any one of SEQ ID NOS: 110-116 and 138. In some embodiments, an anti-HSA AFFIMER® polypeptide comprises the amino acid sequence of any one of SEQ ID NOS: 110-116 and 138. In some embodiments, an anti-HSA AFFIMER® polypeptide comprises an amino acid sequence having 80% to 90% identity to the amino acid sequence of SEQ ID NO: 110. In some embodiments, an anti-HSA AFFIMER® polypeptide comprises an amino acid sequence having 80% to 90% identity to the amino acid sequence of SEQ ID NO: 111. In some embodiments, an anti-HSA AFFIMER® polypeptide comprises an amino acid sequence having 80% to 90% identity to the amino acid sequence of SEQ ID NO: 112. In some embodiments, an anti-HSA AFFIMER® polypeptide comprises an amino acid sequence having 80% to 90% identity to the amino acid sequence of SEQ ID NO: 113. In some embodiments, an anti-HSA AFFIMER® polypeptide comprises an amino acid sequence having 80% to 90% identity to the amino acid sequence of SEQ ID NO: 114. In some embodiments, an anti-HSA AFFIMER® polypeptide comprises an amino acid sequence having 80% to 90% identity to the amino acid sequence of SEQ ID NO: 115. In some embodiments, an anti-HSA AFFIMER® polypeptide comprises an amino acid sequence having 80% to 90% identity to the amino acid sequence of SEQ ID NO: 116. In some embodiments, an anti-HSA AFFIMER® polypeptide comprises an amino acid sequence having 80% to 90% identity to the amino acid sequence of SEQ ID NO: 138.

Anti-HSA AFFIMER® polypeptides provided herein, in some embodiments, are linked to another molecule and extend the half-life of that molecule (e.g., a therapeutic polypeptide). Provided herein is a range of anti-HSA AFFIMER® polypeptides, with a range of binding affinities, for example, that cross-react with other species such as mouse and cynomolgus (cyno) monkey. These anti-HSA AFFIMER® polypeptides, in some embodiments, make up what is referred to as the AFFIMER XT™ platform. These anti-HSA AFFIMER® polypeptides have been shown in in vivo pharmacokinetic (PK) studies to extend, in a controlled manner, the serum half-life of any other AFFIMER® therapeutic to which it is conjugated in a single genetic fusion, for example, that can be made in E. Coli. AFFIMER XT™ can also be used to extend the half-life of other peptide or protein therapeutics.

The term half-life refers to the amount of time it takes for a substance, such as a therapeutic AFFIMER® polypeptide, to lose half of its pharmacologic or physiologic activity or concentration. Biological half-life can be affected by elimination, excretion, degradation (e.g., enzymatic degradation) of the substance, or absorption and concentration in certain organs or tissues of the body. Biological half-life can be assessed, for example, by determining the time it takes for the blood plasma concentration of the substance to reach half its steady state level (“plasma half-life”).

In some embodiments, an anti-HSA AFFIMER® polypeptide extends the serum half-life of a molecule (e.g., a therapeutic polypeptide) in vivo. For example, an anti-HSA AFFIMER® polypeptide may extend the half-life of a molecule by at least 1.2-fold, relative to the half-life of the molecule not linked to an anti-HSA AFFIMER® polypeptide. In some embodiments, an anti-HSA AFFIMER® polypeptide extends the half-life of a molecule by at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, or at least 30-fold, relative to the half-life of the molecule not linked to an anti-HSA AFFIMER® polypeptide. In some embodiments, an anti-HSA AFFIMER® polypeptide extends the half-life of a molecule by 1.2-fold to 5-fold, 1.2-fold to 10-fold, 1.5-fold to 5-fold, 1.5-fold to 10-fold, 2-fold to 5-fold, 2-fold to 10-fold, 3-fold to 5-fold, 3-fold to 10-fold, 15-fold to 5-fold, 4-fold to 10-fold, or 5-fold to 10-fold, relative to the half-life of the molecule not linked to an anti-HSA AFFIMER® polypeptide. In some embodiments, an anti-HSA AFFIMER® polypeptide extends the half-life of a molecule by at least 6 hours, at least 12 hours, at least 24 hours, at least 48 hours, at least 72 hours, at least 96 hours, for example, at least 1 week after in vivo administration, relative to the half-life of the molecule not linked to an anti-HSA AFFIMER® polypeptide.

In some embodiments, an anti-HSA AFFIMER® polypeptide has an extended serum half-life and comprises an amino acid sequence selected from any one of SEQ ID NOS: 117-127, 139, and 140 (Table 3).

TABLE 3
Examples of half-life extension in-line fusion AFFIMER ®
polypeptide sequences

| Name | Sequence | SEQ ID NO: |
|---|---|---|
| AVA04-236 | MIPRGLSEAKPATPEIQEIVDKVKPQLEEKTNETYGKL | 117 |
| XT7 | EAVQYKTQVLA [symbol] STNYYIKVRAGDNK | |
| | YMHLKVFNGP [symbol] ADRVLTGYQVDKNK | |
| | DDELTGFGGGGSGGGGSGGGGSGGGGSGGGGSGGG | |
| | GSMIPRGLSEAKPATPEIQEIVDKVKPQLEEKTNETYG | |
| | KLEAVQYKTQVLA [symbol] STNYYIKVRAGDN | |
| | KYMHLKVFNGP [symbol] ADRVLTGYQVDKNK | |
| | DDELTGF | |
| AVA04-236 | MIPRGLSEAKPATPEIQEIVDKVKPQLEEKTNETYGKL | 118 |
| XT8 | EAVQYKTQVLA [symbol] STNYYIKVRAGDNK | |
| | YMHLKVFNGP [symbol] ADRVLTGYQVDKNK | |
| | DDELTGFAEAAAKEAAAKEAAAKEAAAKEAAAKEA | |
| | AAKMIPRGLSEAKPATPEIQEIVDKVKPQLEEKTNET | |
| | YGKLEAVQYKTQVLA [symbol] STNYYIKVRAG | |
| | DNKYMHLKVFNGP [symbol] ADRVLTGYQVDK | |
| | NKDDELTGF | |
| AVA04-261 | MIPRGLSEAKPATPEIQEIVDKVKPQLEEKTNETYGKL | 119 |
| XT9 | EAVQYKTQVLA [symbol] STNYYIKVRAGDNK | |
| | YMHLKVFNGP [symbol] ADRVLTGYQVDKNKD | |
| | DELTGFGGGGSGGGGSGGGGSGGGGSGGGGSGGGG | |
| | SMIPRGLSEAKPATPEIQEIVDKVKPQLEEKTNETYGK | |
| | LEAVQYKTQVLA [symbol] STNYYIKVRAGDNK | |
| | YMHLKVFNGP [symbol] ADRVLTGYQVDKNKD | |
| | DELTGF | |
| AVA04-261 | MIPRGLSEAKPATPEIQEIVDKVKPQLEEKTNETYGKL | 120 |
| XT10 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAGD | |
| | NKYMHLKVFNGP [symbol] ADRVLTGYQVDKN | |
| | KDDELTGF | |
| AVA04-269 | MIPRGLSEAKPATPEIQEIVDKVKPQLEEKTNETYGKL | 121 |
| XT11 | EAVQYKTQVLA [symbol] STNYYIKVRAGDNK | |
| | YMHLKVFNGP [symbol] ADRVLTGYQVDKNKD | |
| | DELTGFGGGGSGGGGSGGGGSGGGGSGGGGSGGGG | |
| | SMIPRGLSEAKPATPEIQEIVDKVKPQLEEKTNETYGK | |
| | LEAVQYKTQVLA [symbol] STNYYIKVRAGDNK | |
| | YMHLKVFNGP [symbol] ADRVLTGYQVDKNKD | |
| | DELTGF | |
| AVA04-269 | MIPRGLSEAKPATPEIQEIVDKVKPQLEEKTNETYGKL | 122 |
| XT12 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAGD | |
| | NKYMHLKVFNGP [symbol] ADRVLTGYQVDKN | |
| | KDDELTGF | |
| AVA04-251 | MIPRGLSEAKPATPEIQEIVDKVKPQLEEKTNETYGKL | 123 |
| XT14 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAG | |
| | DNKYMHLKVFNGP [symbol] ADRVLTGYQVDK | |
| | NKDDELTGFAEAAAKEAAAKEAAAKEAAAKEAAAK | |
| | EAAAKMIPRGLSEAKPATPEIQEIVDKVKPQLEEKTN | |
| | ETYGKLEAVQYKTQVLA [symbol] STNYYIKVR | |
| | AGDNKYMHLKVFNGP [symbol] ADRVLTGYQV | |
| | DKNKDDELTGF | |
| AVA04-251 | MIPRGLSEAKPATPEIQEIVDKVKPQLEEKTNETYGKL | 124 |
| XT15 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAGD | |
| | NKYMHLKVFNGP [symbol] ADRVLTGYQVDKN | |
| | KDDELTGFAEAAAKEAAAKEAAAKEAAAKEAAAKE | |
| | AAAKMIPRGLSEAKPATPEIQEIVDKVKPQLEEKTNE | |
| | TYGKLEAVQYKTQVLA [symbol] STNYYIKVR | |
| | AGDNKYMHLKVFNGP [symbol] ADRVLTGYQV | |
| | DKNKDDELTGF | |
| AVA04-251 | MIPRGLSEAKPATPEIQEIVDKVKPQLEEKTNETYGKL | 125 |
| XT16 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAG | |
| | DNKYMHLKVFNGP [symbol] ADRVLTGYQVDK | |
| | NKDDELTGFAEAAAKEAAAKEAAAKEAAAKEAAAK | |
| | EAAAKMIPRGLSEAKPATPEIQEIVDKVKPQLEEKTN | |
| | ETYGKLEAVQYKTQVLA [symbol] STNYYIKVR | |
| | AGDNKYMHLKVFNGP [symbol] ADRVLTGYQV | |
| | DKNKDDELTGF | |
| AVA04-251 | MIPRGLSEAKPATPEIQEIVDKVKPQLEEKTNETYGKL | 126 |
| XT14 cys | EAVQYKTQVLA [symbol] TNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAG | |
| | DNKYMHLKVFNGP [symbol] ADRVLTGYQVDK | |
| | NKDDELTGFAEAAAKEAAAKEAAAKEAAAKEAAAK | |
| | EAAAKMIPRGLSEAKPATPEIQEIVDKVKPQLEEKTN | |
| | ETYGKLEAVQYKTQVLA [symbol] STNYYIKVR | |
| | AGDNKYMHLKVFNGP [symbol] ADRVLTGYQV | |
| | DKNKDDELTGFC | |
| AVA04-182 | MIPRGLSEAKPATPEIQEIVDKVKPQLEEKTNETYGKL | 127 |
| XT20 | EAVQYKTQVLA [symbol] STNYYIKVRAGDNKY | |
| | MHLKVFNGP [symbol] ADRVLTGYQVDKNKDD | |
| | ELTGFAEAAAKEAAAKEAAAKEAAAKEAAAKEAAA | |
| | KMIPRGLSEAKPATPEIQEIVDKVKPQLEEKTNETYG | |
| | KLEAVQYKTQVLA [symbol] STNYYIKVRAGDN | |
| | KYMHLKVFNGP [symbol] ADRVLTGYQVDKNK | |
| | DDELTGFAEAAAKEAAAKEAAAKEAAAKEAAAKEA | |
| | AAKMIPRGLSEAKPATPEIQEIVDKVKPQLEEKTNET | |
| | YGKLEAVQYKTQVLA [symbol] STNYYIKVRAG | |
| | DNKYMHLKVFNGP [symbol] ADRVLTGYQVDK | |
| | NKDDELTGF | |
| SQT gly XT28 | MIPRGLSEAKPATPEIQEIVDKVKPQLEEKTNETYGKL | 128 |
| | EAVQYKTQVLA [symbol] STNYYIKVRAGDNK | |
| | YMHLKVFNGP [symbol] ADRVLTGYQVDKNK | |
| | DDELTGFAEAAAKEAAAKEAAAKEAAAKEAAAKEA | |
| | AAKMIPRGLSEAKPATPEIQEIVDKVKPQLEEKTNET | |
| | YGKLEAVQYKTQVLA [symbol] STNYYIKVRA | |
| | GDNKYMHLKVFNGP [symbol] ADRVLTGYQV | |
| | DKNKDDELTGFAEAAAKEAAAKEAAAKEAAAKEAA | |
| | AKEAAAKMIPRGLSEAKPATPEIQEIVDKVKPQLEEK | |
| | TNETYGKLEAVQYKTQVLA [symbol] STNYYIK | |
| | VRAGDNKYMHLKVFNGP [symbol] ADRVLTGY | |
| | QVDKNKDDELTGF | |
| AVA04-251 BH | MIPRGLSEAKPATPEIQEIVDKVKPQLEEKTNETYGKL | 129 |
| | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAG | |
| | DNKYMHLKVFNGP [symbol] ADRVLTGYQVDK | |
| | NKDDELTGF | |
| AVA04-251 BH | MIPRGLSEAKPATPEIQEIVDKVKPQLEEKTNETYGKL | 130 |
| cys | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAG | |
| | DNKYMHLKVFNGP [symbol] ADRVLTGYQVDK | |
| | NKDDELTGFC | |
| HSA-41 BK | MIPRGLSEAKPATPEIQEIVDKVKPQLEEKTNETYGKL | 131 |
| | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAGD | |
| | NKYMHLKVFNGP [symbol] ADRVLTGYQVDKN | |
| | KDDELTGF | |
| HSA-41 DI | MIPRGLSEAKPATPEIQEIVDKVKPQLEEKTNETYGKL | 132 |
| | EAVQYKTQVLA [symbol] STNYYIKVRAGDNK | |
| | YMHLKVFNGP [symbol] ADRVLTGYQVDKNKD | |
| | DELTGFGGGGSGGGGSGGGGSGGGGSGGGGSGGGG | |
| | SMIPRGLSEAKPATPEIQEIVDKVKPQLEEKTNETYGK | |
| | LEAVQYKTQVLA [symbol] STNYYIKVRAGDNK | |
| | YMHLKVFNGP [symbol] ADRVLTGYQVDKNKD | |
| | DELTGF | |
| AVA04-251 | MIPRGLSEAKPATPEIQEIVDKVKPQLEEKTNETYGKL | 139 |
| XT60 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLA [symbol] STNYYIKVRAG | |
| | DNKYMHLKVFNGP [symbol] ADRVLTGYQVDK | |
| | NKDDELTGFAEAAAKEAAAKEAAAKEAAAKEAAAK | |
| | EAAAKMIPRGLSEAKPATPEIQEIVDKVKPQLEEKTN | |
| | ETYGKLEAVQYKTQVLADWWQAKWPHSTNYYIKV | |
| | RAGDNKYMHLKVFNGPYKVHQSSGGADRVLTGYQ | |
| | VDKNKDDELTGF | |
| AVA04-251 | MIPRGLSEAKPATPEIQEIVDKVKPQLEEKTNETYGKL | 140 |
| XT61 | EAVQYKTQVLA [symbol] STNYYIKVRAGDNK | |
| | YMHLKVFNGP [symbol] ADRVLTGYQVDKNKD | |
| | DELTGFAEAAAKEAAAKEAAAKEAAAKEAAAKEAA | |
| | AKMIPRGLSEAKPATPEIQEIVDKVKPQLEEKTNETY | |
| | GKLEAVQYKTQVLADWWQAKWPHSTNYYIKVRAG | |
| | DNKYMHLKVFNGPYKVHQSSGGADRVLTGYQVDK | |
| | NKDDELTGFAEAAAKEAAAKEAAAKEAAAKEAAAK | |
| | EAAAKMIPRGLSEAKPATPEIQEIVDKVKPQLEEKTN | |
| | ETYGKLEAVQYKTQVLA [symbol] STNYYIKVR | |
| | AGDNKYMHLKVFNGP [symbol] ADRVLTGYQV | |
| | DKNKDDELTGF | |

In some embodiments, an anti-HSA AFFIMER® polypeptide comprises an amino acid sequence having at least 80% or at least 90% identity to the amino acid sequence of any one of SEQ ID NOS: 117-127, 139, and 140. In some embodiments, an anti-HSA AFFIMER® polypeptide comprises an amino acid sequence having at least 80% identity to the amino acid sequence of SEQ ID NO: 117. In some embodiments, an anti-HSA AFFIMER® polypeptide comprises an amino acid sequence having at least 80% identity to the amino acid sequence of SEQ ID NO: 118. In some embodiments, an anti-HSA AFFIMER® polypeptide comprises an amino acid sequence having at least 80% identity to the amino acid sequence of SEQ ID NO: 119. In some embodiments, an anti-HSA AFFIMER® polypeptide comprises an amino acid sequence having at least 80% identity to the amino acid sequence of SEQ ID NO: 120. In some embodiments, an anti-HSA AFFIMER® polypeptide comprises an amino acid sequence having at least 80% identity to the amino acid sequence of SEQ ID NO: 121. In some embodiments, an anti-HSA AFFIMER® polypeptide comprises an amino acid sequence having at least 80% identity to the amino acid sequence of SEQ ID NO: 122. In some embodiments, an anti-HSA AFFIMER® polypeptide comprises an amino acid sequence having at least 80% identity to the amino acid sequence of SEQ ID NO: 123. In some embodiments, an anti-HSA AFFIMER® polypeptide comprises an amino acid sequence having at least 80% identity to the amino acid sequence of SEQ ID NO: 124. In some embodiments, an anti-HSA AFFIMER® polypeptide comprises an amino acid sequence having at least 80% identity to the amino acid sequence of SEQ ID NO: 125. In some embodiments, an anti-HSA AFFIMER® polypeptide comprises an amino acid sequence having at least 80% identity to the amino acid sequence of SEQ ID NO: 126. In some embodiments, an anti-HSA AFFIMER® polypeptide comprises an amino acid sequence having at least 80% identity to the amino acid sequence of SEQ ID NO: 127. In some embodiments, an anti-HSA AFFIMER® polypeptide comprises an amino acid sequence having at least 80% identity to the amino acid sequence of SEQ ID NO: 139. In some embodiments, an anti-HSA AFFIMER® polypeptide comprises an amino acid sequence having at least 80% identity to the amino acid sequence of SEQ ID NO: 140.

In some embodiments, an anti-HSA AFFIMER® polypeptide comprises an amino acid sequence having at least 90% identity to the amino acid sequence of SEQ ID NO: 117. In some embodiments, an anti-HSA AFFIMER® polypeptide comprises an amino acid sequence having at least 90% identity to the amino acid sequence of SEQ ID NO: 118. In some embodiments, an anti-HSA AFFIMER® polypeptide comprises an amino acid sequence having at least 90% identity to the amino acid sequence of SEQ ID NO: 119. In some embodiments, an anti-HSA AFFIMER® polypeptide comprises an amino acid sequence having at least 90% identity to the amino acid sequence of SEQ ID NO: 120. In some embodiments, an anti-HSA AFFIMER® polypeptide comprises an amino acid sequence having at least 90% identity to the amino acid sequence of SEQ ID NO: 121. In some embodiments, an anti-HSA AFFIMER® polypeptide comprises an amino acid sequence having at least 90% identity to the amino acid sequence of SEQ ID NO: 122. In some embodiments, an anti-HSA AFFIMER® polypeptide comprises an amino acid sequence having at least 90% identity to the amino acid sequence of SEQ ID NO: 123. In some embodiments, an anti-HSA AFFIMER® polypeptide comprises an amino acid sequence having at least 90% identity to the amino acid sequence of SEQ ID NO: 124. In some embodiments, an anti-HSA AFFIMER® polypeptide comprises an amino acid sequence having at least 90% identity to the amino acid sequence of SEQ ID NO: 125. In some embodiments, an anti-HSA AFFIMER® polypeptide comprises an amino acid sequence having at least 90% identity to the amino acid sequence of SEQ ID NO: 126. In some embodiments, an anti-HSA AFFIMER® polypeptide comprises an amino acid sequence having at least 90% identity to the amino acid sequence of SEQ ID NO: 127. In some embodiments, an anti-HSA AFFIMER® polypeptide comprises an amino acid sequence having at least 90% identity to the amino acid sequence of SEQ ID NO: 139. In some embodiments, an anti-HSA AFFIMER® polypeptide comprises an amino acid sequence having at least 90% identity to the amino acid sequence of SEQ ID NO: 140.

In some embodiments, an anti-HSA AFFIMER® polypeptide comprises an amino acid sequence having 80% to 90% identity to the amino acid sequence of any one of SEQ ID NOS: 117-127, 139, and 140. In some embodiments, an anti-HSA AFFIMER® polypeptide comprises the amino acid sequence of any one of SEQ ID NOS: 117-127, 139, and 140. In some embodiments, an anti-HSA AFFIMER® polypeptide comprises an amino acid sequence having 80% to 90% identity to the amino acid sequence of SEQ ID NO: 117. In some embodiments, an anti-HSA AFFIMER® polypeptide comprises an amino acid sequence having 80% to 90% identity to the amino acid sequence of SEQ ID NO: 118. In some embodiments, an anti-HSA AFFIMER® polypeptide comprises an amino acid sequence having 80% to 90% identity to the amino acid sequence of SEQ ID NO: 119. In some embodiments, an anti-HSA AFFIMER® polypeptide comprises an amino acid sequence having 80% to 90% identity to the amino acid sequence of SEQ ID NO: 120. In some embodiments, an anti-HSA AFFIMER® polypeptide comprises an amino acid sequence having 80% to 90% identity to the amino acid sequence of SEQ ID NO: 121. In some embodiments, an anti-HSA AFFIMER® polypeptide comprises an amino acid sequence having 80% to 90% identity to the amino acid sequence of SEQ ID NO: 122. In some embodiments, an anti-HSA AFFIMER® polypeptide comprises an amino acid sequence having 80% to 90% identity to the amino acid sequence of SEQ ID NO: 123. In some embodiments, an anti-HSA AFFIMER® polypeptide comprises an amino acid sequence having 80% to 90% identity to the amino acid sequence of SEQ ID NO: 124. In some embodiments, an anti-HSA AFFIMER® polypeptide comprises an amino acid sequence having 80% to 90% identity to the amino acid sequence of SEQ ID NO: 125. In some embodiments, an anti-HSA AFFIMER® polypeptide comprises an amino acid sequence having 80% to 90% identity to the amino acid sequence of SEQ ID NO: 126. In some embodiments, an anti-HSA AFFIMER® polypeptide comprises an amino acid sequence having 80% to 90% identity to the amino acid sequence of SEQ ID NO: 127. In some embodiments, an anti-HSA AFFIMER® polypeptide comprises an amino acid sequence having 80% to 90% identity to the amino acid sequence of SEQ ID NO: 139. In some embodiments, an anti-HSA AFFIMER® polypeptide comprises an amino acid sequence having 80% to 90% identity to the amino acid sequence of SEQ ID NO: 140.

### Polypeptides

A polypeptide is a polymer of amino acids (naturally-occurring or non-naturally occurring, e.g., amino acid analogs) of any length. The terms “polypeptide” and “peptide” are used interchangeably herein unless noted otherwise. A protein is one example of a polypeptide. It should be understood that a polypeptide may be linear or branched, it may comprise naturally-occurring and/or non-naturally-occurring (e.g., modified) amino acids, and/or it may include non-amino acids (e.g., interspersed throughout the polymer). A polypeptide, as provided herein, may be modified (e.g., naturally or non-naturally), for example, via disulfide bond formation, glycosylation, lipidation, acetylation, phosphorylation, or conjugation with a labeling component. Polypeptides, in some instances, may contain at least one analog of an amino acid (including, for example, unnatural amino acids) and/or other modifications.

An amino acid (also referred to as an amino acid residue) participates in peptide bonds of a polypeptide. In general, the abbreviations used herein for designating the amino acids are based on recommendations of the IUPAC-IUB Commission on Biochemical Nomenclature (see Biochemistry (1972) 11:1726-1732). For instance, Met, Ile, Leu, Ala and Gly represent “residues” of methionine, isoleucine, leucine, alanine and glycine, respectively. A residue is a radical derived from the corresponding α-amino acid by eliminating the OH portion of the carboxyl group and the H portion of the α-amino group. An amino acid side chain is that part of an amino acid exclusive of the —CH(NH2)COOH portion, as defined by K. D. Kopple, “Peptides and Amino Acids”, W. A. Benjamin Inc., New York and Amsterdam, 1966, pages 2 and 33.

Amino acids used herein, in some embodiments, are naturally-occurring amino acids found in proteins, for example, or the naturally-occurring anabolic or catabolic products of such amino acids that contain amino and carboxyl groups. Examples of amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan, and those amino acids and amino acid analogs that have been identified as constituents of peptidylglycan bacterial cell walls.

Amino acids having basic sidechains include Arg, Lys and His. Amino acids having acidic sidechains include Glu and Asp. Amino acids having neutral polar sidechains include Ser, Thr, Asn, Gln, Cys and Tyr. Amino acids having neutral non-polar sidechains include Gly, Ala, Val, Ile, Leu, Met, Pro, Trp and Phe. Amino acids having non-polar aliphatic sidechains include Gly, Ala, Val, Ile and Leu. Amino acids having hydrophobic sidechains include Ala, Val, Ile, Leu, Met, Phe, Tyr and Trp. Amino acids having small hydrophobic sidechains include Ala and Val. Amino acids having aromatic sidechains include Tyr, Trp and Phe.

The term amino acid includes analogs, derivatives and congeners of any specific amino acid referred to herein; for instance, the AFFIMER® polypeptides (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid. Other naturally-occurring amino acid metabolites or precursors having side chains that are suitable herein will be recognized by those skilled in the art and are included in the scope of the present disclosure.

Also included herein are the (D) and (L) stereoisomers of such amino acids when the structure of the amino acid admits of stereoisomeric forms. The configuration of the amino acids and amino acids herein are designated by the appropriate symbols (D), (L) or (DL); furthermore, when the configuration is not designated the amino acid or residue can have the configuration (D), (L) or (DL). It will be noted that the structure of some of the compounds of the present disclosure includes asymmetric carbon atoms. It is to be understood accordingly that the isomers arising from such asymmetry are included within the scope of the present disclosure. Such isomers can be obtained in substantially pure form by classical separation techniques and by sterically controlled synthesis. For the purposes of this disclosure, unless expressly noted to the contrary, a named amino acid shall be construed to include both the (D) or (L) stereoisomers.

Percent identity, in the context of two or more nucleic acids or polypeptides, refers to two or more sequences or subsequences that are the same (identical/100% identity) or have a specified percentage (e.g., at least 70% identity) of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the present disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A conservative amino acid substitution is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the present disclosure do not abrogate the binding of the polypeptide, soluble protein, or antibody containing the amino acid sequence, to the target binding site. Methods of identifying amino acid conservative substitutions that do not eliminate binding are well-known in the art.

Herein, it should be understood that an isolated molecule (e.g., polypeptide (e.g., soluble protein, antibody, etc.), polynucleotide (e.g., vector), cell, or other composition) is in a form not found in nature. Isolated molecules, for example, have been purified to a degree that is not possible in nature.

In some embodiments, an isolated molecule (e.g., polypeptide (e.g., soluble protein, antibody, etc.), polynucleotide (e.g., vector), cell, or other composition) is substantially pure, which refer to an isolated molecule that is at least 50% pure (e.g., free from 50% of contaminants associated with the unpurified form of the molecule), at least 90% pure, at least 95% pure, at least 98% pure, or at least 99% pure.

Conjugates, Including Polypeptide Fusions

The verb conjugate (used interchangeably with the verb link) herein refers to the joining together of two or more molecules (e.g., polypeptides and/or chemical moieties) to form another molecule. Thus, one molecule (e.g., an anti-HSA AFFIMER® polypeptide) conjugated to another molecule (e.g., another AFFIMER® polypeptide, drug molecule, or other therapeutic protein or nucleic acid) forms a conjugate. The joining of two or more molecules can be, for example, through a non-covalent bond or a covalent bond. For example, an anti-HSA AFFIMER® polypeptide linked directly or indirectly to a chemical moiety or to another polypeptide (e.g., a heterologous polypeptide) forms a conjugate, as provided herein. Non-limiting examples of conjugates include chemical conjugates (e.g., joined through “click” chemistry or another chemical reaction) and fusions (two molecules linked by contiguous peptide bonds). In some embodiments, a conjugate is a fusion polypeptide, for example, a fusion protein. In some embodiments, an anti-HSA AFFIMER® polypeptide is conjugated to two or more other molecules. For example, dual (or multi) mode of action drug conjugates may be conjugated to an anti-HSA AFFIMER® polypeptide of the present disclosure. Such dual mode of action drug conjugates include those of the TMAC (Tumor Microenvironment-Activated Conjugates) platform (see, e.g., avacta.com/therapeutics/tmac-affimer-drug-conjugates).

A fusion polypeptide (e.g., fusion protein) is a polypeptide comprising at least two domains (e.g., protein domains) encoded by a polynucleotide comprising nucleotide sequences of at least two separate molecules (e.g., two genes). In some embodiments, a polypeptide comprises a heterologous polypeptide covalently linked (to an amino acid of the polypeptide) through an amide bond to form a contiguous fusion polypeptide (e.g., fusion protein). In some embodiments, the heterologous polypeptide comprises a therapeutic polypeptide. In some embodiments, an anti-HSA AFFIMER® polypeptide is conjugated to a heterologous polypeptide through contiguous peptide bonds at the C-terminus or N-terminus of the anti-HSA AFFIMER® polypeptide.

A linker is a molecule inserted between a first polypeptide (e.g., as AFFIMER® polypeptide) and a second polypeptide (e.g., another AFFIMER® polypeptide, an Fc domain, a ligand binding domain, etc). A linker may be any molecule, for example, one or more nucleotides, amino acids, chemical functional groups. In some embodiments, the linker is a peptide linker (e.g., two or more amino acids). Linkers should not adversely affect the expression, secretion, or bioactivity of the polypeptides. In some embodiments, linkers are not antigenic and do not elicit an immune response. An immune response includes a response from the innate immune system and/or the adaptive immune system. Thus, an immune response may be a cell-mediate response and/or a humoral immune response. The immune response may be, for example, a T cell response, a B cell response, a natural killer (NK) cell response, a monocyte response, and/or a macrophage response. Other cell response are contemplated herein.

In some embodiments, linkers are non-protein-coding.

In some embodiments, a conjugate comprises an AFFIMER® polypeptide linked to a therapeutic or diagnostic molecule. In some embodiments, a conjugate comprises an AFFIMER® polypeptide linked to another protein, a nucleic acid, a drug, or other small molecule or macromolecule.

Any conjugation method may be used, or readily adapted, for joining a molecule to an AFFIMER® polypeptide of the present disclosure, including, for example, the methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407.

### Therapeutics

In some embodiments, the therapeutic molecule is for the treatment of an autoimmune disease (a condition in which a subject's immune system mistaken attacks his/her body). Non-limiting examples of autoimmune diseases include myasthenia gravis, pemphigus vulgaris, neuromyelitis optica, Guillain-Barre syndrome, rheumatoid arthritis, systemic lupus erythematosus (lupus), idiopathic thrombocytopenic purpura, thrombotic thrombocytopenic purpura, antiphospholipid syndrome (APS), autoimmune urticarial, chronic inflammatory demyelinating polyneuropathy (CIDP), psoriasis, Goodpasture's syndrome, Graves' disease, inflammatory bowel disease, Crohn's disease, Sjorgren's syndrome, hemolytic anemia, neutropenia, paraneoplastic cerebellar degeneration, paraproteinemic polyneuropathies, primary biliary cirrhosis, stiff person syndrome, vitiligo, warm idiopathic haemolytic anaemia, multiple sclerosis, type 1 diabetes mellitus, Hashimoto's thyroiditis, Myasthenia gravis, autoimmune vasculitis, pernicus anemia, and celiac disease. Other autoimmune diseases are contemplated herein.

In some embodiments, the therapeutic molecule is for the treatment of a cancer. Non-limiting examples of cancers include skin cancer (e.g., melanoma or non-melanom, such as basal cell or squamous cell), lung cancer, prostate cancer, breast cancer, colorectal cancer, kidney (renal) cancer, bladder cancer, non-Hodgkin's lymphoma, thyroid cancer, endometrial cancer, exocrine cancer, and pancreatic cancer. Other cancers are contemplated herein.

In some embodiments, an AFFIMER® polypeptide is linked to a therapeutic molecule. Herein, a therapeutic molecule may be used, for example, to prevent and/or treat a disease in a subject, such as a human subject or other animal subject. The term treat, as known in the art, refers to the process of alleviating at least one symptom associated with a disease. A symptom may be a physical, mental, or pathological manifestation of a disease. Symptoms associated with various diseases are known. To treat or prevent a particular condition, a conjugate as provided herein (e.g., an anti-HSA AFFIMER® polypeptide linked to a therapeutic molecule) should be administered in an effective amount, which can be any amount used to treat or prevent the condition. Thus, in some embodiments, an effective amount is an amount used to alleviate a symptom associated with the particular disease being treated. Methods are known for determining effective amounts of various therapeutic molecules, for example.

A subject may be any animal (e.g., a mammal), including, but not limited to, humans, non-human primates, canines, felines, and rodents. A “patient” refers to a human subject.

In some embodiments, an anti-HSA AFFIMER® polypeptide is linked to an agonist of a particular molecule (e.g., receptor) of interest. In other embodiments, an anti-HSA AFFIMER® polypeptide is linked to an antagonist of a particular molecule of interest. An agonist herein refers to a molecule that binds to and activates another molecule to produce a biological response. By contrast, an antagonist blocks the action of the agonist, and an inverse agonist causes an action opposite to that of the agonist. Thus, an antagonist herein refers to a molecule that binds to and deactivates or prevents activation of another molecule.

In some embodiments, an AFFIMER® polypeptide is considered “pharmaceutically acceptable,” and in some embodiments, is formulated with a pharmaceutically-acceptable excipient. A molecule or other substance/agent is considered “pharmaceutically acceptable” if it is approved or approvable by a regulatory agency of the Federal government or a state government or listed in the U.S. Pharmacopeia or other generally recognized pharmacopeia for use in animals, including humans. An excipient may be any inert (inactive), non-toxic agent, administered in combination with an AFFIMER® polypeptide. Non-limiting examples of excipients include buffers (e.g., sterile saline), salts, carriers, preservatives, fillers, and coloring agents.

Therapeutic molecules for use herein include, for example, those recognized in the official United States Pharmacopeia, official Homeopathic Pharmacopeia of the United States, official National Formulary, or any supplement thereof, and include, but are not limited, to small molecules chemicals/drugs, polynucleotides (e.g., RNA interference molecules, such as miRNA, siRNA, shRNA, and antisense RNA), and polypeptides (e.g., antibodies). Classes of therapeutic molecules that may be used as provided herein include, but are not limited to, recombinant proteins, antibodies, cytotoxic agents, anti-metabolites, alkylating agents, antibiotics, growth factors (e.g., erythropoetin, granulocyte colony-stimulating factor (G-CSF), granulocyte-macrophage colony-stimulating factor (GM-CSF), keratinocyte growth factor)), cytokines, chemokines, interferons (e.g., interferon-alpha, interferon-beta, interferon-gamma), blood factors (e.g., factor VIII, factor Vila, factor IX, thrombin, antithrombin), anti-mitotic agents, toxins, apoptotic agents, (e.g., DNA alkylating agents), topoisomerase inhibitors, endoplasmic reticulum stress inducing agents, platinum compounds, antimetabolites, vincalkaloids, taxanes, epothilones, enzyme inhibitors, receptor antagonists, tyrosine kinase inhibitors, radiosensitizers, chemotherapeutic combination therapies, receptor traps, receptor ligands, angiogenic agents, anti-angiogenic agents, anti-coagulants and thrombolytics (e.g., tissue plasminogen activator, hirudin, protein C), neurotransmitters, erythropoiesis-stimulating agents, insulin, growth hormones (e.g., human growth hormone (hGH), follicle-stimulating hormone), metabolic hormones (e.g., incretins), recombinant IL-1 receptor antagonists, and bispecific T-cell engaging molecules)(BITEs®).

Specific examples of therapeutic molecules to which an anti-human FcRn AFFIMER® polypeptide may be linked (e.g., to extend the half-life of the molecules) includes fibroblast growth factor 21 (FGF21), insulin, insulin receptor peptide, GIP (glucose-dependent insulinotropic polypeptide), bone morphogenetic protein 9 (BMP-9), amylin, peptide YY (PYY3-36), pancreatic polypeptide (PP), interleukin 21 (IL-21), glucagon-like peptide 1 (GLP-1), Plectasin, Progranulin, Osteocalcin (OCN), Apelin, GLP-1, Exendin 4, adiponectin, IL-1Ra (Interleukin 1 Receptor Antagonist), VIP (vasoactive intestinal peptide), PACAP (Pituitary adenylate cyclase-activating polypeptide), leptin, INGAP (islet neogenesis associated protein), BMP (bone morphogenetic protein), and osteocalcin (OCN).

Antibodies

In some embodiments, a heterologous polypeptide to which an anti-HSA AFFIMER® polypeptide is linked is an antibody (e.g., a variable region of an antibody). Thus, the present disclosure, in some embodiments, provides an AFFIMER® polypeptide-antibody fusion protein. In some embodiments, an AFFIMER® polypeptide-antibody fusion protein comprises a full length antibody comprising, for example, at least one AFFIMER® polypeptide sequence appended to the C-terminus or N-terminus of at least one of its VH and/or VL chains (at least one chain of the assembled antibody forms a fusion protein with an AFFIMER® polypeptide). AFFIMER® polypeptide-antibody fusion proteins, in some embodiments, comprise at least one AFFIMER® polypeptide and an antigen binding site or variable region of an antibody fragment.

An antibody is an immunoglobulin molecule that recognizes and specifically binds a target, such as a polypeptide (e.g., peptide or protein), polynucleotide, carbohydrate, lipid, or a combination of any of the foregoing, through at least one antigen-binding site. The antigen-binding site, in some embodiments, is within the variable region of the immunoglobulin molecule. Antibodies include polyclonal antibodies, monoclonal antibodies, antibody fragments (such as Fab, Fab′, F(ab′)2, and Fv fragments), single chain Fv (scFv) antibodies provided those fragments have been formatted to include an Fc or other FcγRIII binding domain, multispecific antibodies, bispecific antibodies, monospecific antibodies, monovalent antibodies, chimeric antibodies, humanized antibodies, human antibodies, fusion proteins comprising an antigen-binding site of an antibody (formatted to include an Fc or other FcγRIII binding domain), and any other modified immunoglobulin molecule comprising an antigen-binding site as long as the antibodies exhibit the desired biological activity.

An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu.

A variable region of an antibody can be a variable region of an antibody light chain or a variable region of an antibody heavy chain, either alone or in combination. Generally, the variable region of heavy and light chains each consist of four framework regions (FR) and three complementarity determining regions (CDRs), also known as hypervariable regions. The CDRs in each chain are held together in close proximity by the framework regions and, with the CDRs from the other chain, contribute to the formation of the antigen-binding sites of the antibody. There are at least two techniques for determining CDRs: (1) an approach based on cross-species sequence variability (Kabat et al., 1991, Sequences of Proteins of Immunological Interest, 5th Edition, National Institutes of Health, Bethesda Md.), and (2) an approach based on crystallographic studies of antigen-antibody complexes (Al Lazikani et al., 1997, J. Mol. Biol., 273:927-948). In addition, combinations of these two approaches are sometimes used in the art to determine CDRs.

Humanized antibodies are forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the Fv framework region residues of a human immunoglobulin are replaced with the corresponding residues in an antibody from a non-human species. A humanized antibody can be further modified by the substitution of additional residues either in the Fv framework region and/or within the replaced non-human residues to refine and optimize antibody specificity, affinity, and/or binding capability. A humanized antibody may comprise variable domains containing all or substantially all of the CDRs that correspond to the non-human immunoglobulin whereas all or substantially all of the framework regions are those of a human immunoglobulin sequence. In some embodiments, the variable domains comprise the framework regions of a human immunoglobulin sequence. In some embodiments, the variable domains comprise the framework regions of a human immunoglobulin consensus sequence. The humanized antibody can also comprise at least a portion of an immunoglobulin constant region or domain (Fc), typically that of a human immunoglobulin. A humanized antibody is usually considered distinct from a chimeric antibody.

An epitope (also referred to as an antigenic determinant) is a portion of an antigen capable of being recognized and specifically bound by a particular antibody, a particular AFFIMER® polypeptide, or other particular binding domain. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation.

The term “specifically binds to” or is “specific for” refers to measurable and reproducible interactions such as binding between a target and an AFFIMER® polypeptide, antibody or other binding partner, which is determinative of the presence of the target in the presence of a heterogeneous population of molecules including biological molecules. For example, an AFFIMER® polypeptide that specifically binds to a target is an AFFIMER® polypeptide that binds this target with greater affinity, avidity (if multimeric formatted), more readily, and/or with greater duration than it binds to other targets.

Non-limiting examples of antibodies that may be conjugated to an anti-HSA an AFFIMER® polypeptide of the present disclosure 3F8, 8H9, abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, anatumomab mafenatox, andecaliximab, anetumab ravtansine, anifrolumab, anrukinzumab (IMA-638), apolizumab, aprutumab ixadotin, arcitumomab, ascrinvacumab, aselizumab, atezolizumab, atidortoxumab, atinumab, atorolimumab, avelumab, azintuxizumab vedotin, bapineuzumab, basiliximab, bavituximab, BCD-100, bectumomab, begelomab, belantamab mafodotin, belimumab, bemarituzumab, benralizumab, berlimatoxumab, bermekimab, bersanlimab, bertilimumab, besilesomab, bevacizumab, bezlotoxumab, biciromab, bimagrumab, bimekizumab, birtamimab, bivatuzumab mertansine, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, capromab pendetide, carlumab, carotuximab, catumaxomab, cBR96-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, CR6261, crenezumab, crizanlizumab, crotedumab, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, diridavumab, domagrozumab, dorlimomab aritox, dostarlimab, drozitumab, DS-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab vedotin, enavatuzumab, enfortumab vedotin, enlimomab pegol, enoblituzumab, enokizumab, enoticumab, ensituximab, epitumomab cituxetan, epratuzumab, eptinezumab, erenumab, erlizumab, ertumaxomab, etaracizumab, etigilimab, etrolizumab, evinacumab, evolocumab, exbivirumab, fanolesomab, faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imgatuzumab, inclacumab, indatuximab ravtansine, indusatumab vedotin, inebilizumab, infliximab, inolimomab, inotuzumab ozogamicin, intetumumab, iomab-b, ipilimumab, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, lilotomab satetraxetan, lintuzumab, lirilumab, lodelcizumab, lokivetmab, loncastuximab tesirine, lorvotuzumab mertansine, losatuxizumab vedotin, lucatumumab, lulizumab pegol, lumiliximab, lumretuzumab, lupartumab amadotin, lutikizumab, mapatumumab, margetuximab, marstacimab, maslimomab, matuzumab, mavrilimumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, pdr001, pembrolizumab, pemtumomab, perakizumab, pertuzumab, pexelizumab, pidilizumab, pinatuzumab vedotin, pintumomab, placulumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab, ravagalimab, ravulizumab, raxibacumab, refanezumab, regavirumab, relatlimab, remtolumab, reslizumab, rilotumumab, rinucumab, risankizumab, rituximab, rivabazumab pegol, rmab, robatumumab, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sarilumab, satralizumab, satumomab pendetide, secukinumab, selicrelumab, seribantumab, setoxaximab, setrusumab, sevirumab, SGN-CD19A, SHP647, sibrotuzumab, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, talacotuzumab, talizumab, tamtuvetmab, tanezumab, taplitumomab paptox, tarextumab, tavolimab, tefibazumab, telimomab aritox, telisotuzumab vedotin, tenatumomab, teneliximab, teplizumab, tepoditamab, teprotumumab, tesidolumab, tetulomab, tezepelumab, TGN1412, tibulizumab, tigatuzumab, tildrakizumab, timigutuzumab, timolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab (IMAB362, claudiximab), and zolimomab aritox.

Other Therapeutic Molecules

Non-limiting examples of cytokines include IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, CD80, B70, TNF beta, LT-beta, CD-40 ligand, Fas-ligand, TGF-beta, IL-1alpha and IL-1 beta.

Non-limiting examples of chemokines include IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MIP-1alpha, MIP-1 beta, MDC, TECK, TARC, RANTES, HCC-1, HCC-4, DC-CK1, MIP-3 alpha, MIP-3 beta, MCP-1-5, eotaxin, Eotaxin-2, 1-309, MPIF-1, 6Ckine, CTACK, MEC, lymphotactin and fractalkine.

Non-limiting examples of DNA alkylating agents include nitrogen mustards, such as mechlorethamine, cyclophosphamide (ifosfamide, trofosfamide), chlorambucil (melphalan, prednimustine), bendamustine, uramustine and estramustine; nitrosoureas, such as carmustine (bcnu), lomustine (semustine), fotemustine, nimustine, ranimustine and streptozocin; alkyl sulfonates, such as busulfan (mannosulfan, treosulfan); aziridines, such as carboquone, thiotepa, triaziquone, triethylenemelamine; hydrazines (procarbazine); triazenes such as dacarbazine and temozolomide; altretamine and mitobronitol.

Non-limiting examples of topoisomerase I inhibitors include campothecin derivatives including CPT-11 (irinotecan), SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as described in Pommier Y. (2006) Nat. Rev. Cancer 6(10):789-802 and U.S. Patent Publication No. 200510250854; protoberberine alkaloids and derivatives thereof including berberrubine and coralyne as described in Li et al. (2000) Biochemistry 39(24):7107-7116 and Gatto et al. (1996) Cancer Res. 15(12):2795-2800; phenanthroline derivatives including benzo[i]phenanthridine, nitidine, and fagaronine as described in Makhey et al. (2003) Bioorg. Med. Chem. 11 (8): 1809-1820; terbenzimidazole and derivatives thereof as described in Xu (1998) Biochemistry 37(10):3558-3566; and anthracycline derivatives including doxorubicin, daunorubicin, and mitoxantrone as described in Foglesong et al. (1992) Cancer Chemother. Pharmacol. 30(2):123-]25, Crow et al. (1994) J. Med. Chem. 37(19):31913194, and Crespi et al. (1986) Biochem. Biophys. Res. Commun. 136(2):521-8. Topoisomerase II inhibitors include but are not limited to Etoposide and teniposide. Dual topoisomerase I and II inhibitors include, but are not limited to, saintopin and other naphthecenediones, DACA and other Acridine-4-carboxamindes, intoplicine and other benzopyridoindoles, tas-103 and other 7h-indeno[2,1-c]quinoline-7-ones, pyrazoloacridine, XR 11576 and other benzophenazines, XR 5944 and other Dimeric compounds, 7-oxo-7H-dibenz[f,ij]Isoquinolines and 7-oxo-7H-benzo[e]perimidines, and anthracenyl-amino Acid Conjugates as described in Denny and Baguley (2003) Curr. Top. Med. Chem. 3(3):339-353.

Some agents inhibit topoisomerase II and have DNA intercalation activity such as, but not limited to, anthracyclines (aclarubicin, daunorubicin, doxorubicin, epirubicin, idarubicin, amrubicin, pirarubicin, valrubicin, zorubicin) and antracenediones (mitoxantrone and pixantrone).

Non-limiting examples of of endoplasmic reticulum stress inducing agents include dimethyl-celecoxib (DMC), nelfinavir, celecoxib, and boron radiosensitizers (i.e. velcade (bortezomib)).

Non-limiting examples of platinum-based compound include carboplatin, cisplatin, nedaplatin, oxaliplatin, triplatin tetranitrate, satraplatin, aroplatin, lobaplatin, and JM-216. (see McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

Non-limiting examples of antimetabolite agents include folic acid-based, e.g., dihydrofolate reductase inhibitors, such as aminopterin, methotrexate and pemetrexed; thymidylate synthase inhibitors, such as raltitrexed, pemetrexed; purine based, e.g., an adenosine deaminase inhibitor, such as pentostatin, a thiopurine, such as thioguanine and mercaptopurine, a halogenated/ribonucleotide reductase inhibitor, such as cladribine, clofarabine, fludarabine, or a guanine/guanosine: thiopurine, such as thioguanine; or pyrimidine based, e.g., cytosine/cytidine: hypomethylating agent, such as azacitidine and decitabine, a dna polymerase inhibitor, such as cytarabine, a ribonucleotide reductase inhibitor, such as gemcitabine, or a thymine/thymidine: thymidylate synthase inhibitor, such as a fluorouracil (5-FU). Equivalents to 5-FU include prodrugs, analogs and derivative thereof such as 5′-deoxy-5-fluorouridine (doxifluoroidine), 1-tetrahydrofuranyl-5-fluorouracil (FTORAFUR®), capecitabine (XELODA®), S-I (MBMS-247616, consisting of tegafur and two modulators, a 5-chloro-2,4-dihydroxypyridine and potassium oxonate), ralititrexed (TOMUDEX®), no latrexed (Thymitaq, AG337), LY231514 and ZD9331, as described for example in Papamicheal (1999) The Oncologist 4:478-487.

Non-limiting examples of vincalkaloids vinblastine, vincristine, vinflunine, vindesine and vinorelbine.

Non-limiting examples of taxanes include docetaxel, larotaxel, ortataxel, paclitaxel and tesetaxel. an example of an epothilone is iabepilone.

Non-limiting examples of enzyme inhibitors include farnesyltransferase inhibitors (tipifamib); CDK inhibitor (alvocidib, seliciclib); proteasome inhibitor (bortezomib); phosphodiesterase inhibitor (anagrelide; rolipram); IMP dehydrogenase inhibitor (tiazofurine); and lipoxygenase inhibitor (masoprocol). Examples of receptor antagonists include but are not limited to ERA (atrasentan); retinoid X receptor (bexarotene); and a sex steroid (testolactone).

Non-limiting examples of tyrosine kinase inhibitors include inhibitors to ErbB: HER1/EGFR (erlotinib, gefitinib, lapatinib, vandetanib, sunitinib, neratinib); HER2/neu (lapatinib, neratinib); RTK class III: C-kit (axitinib, sunitinib, sorafenib), FLT3 (lestaurtinib), PDGFR (axitinib, sunitinib, sorafenib); and VEGFR (vandetanib, semaxanib, cediranib, axitinib, sorafenib); bcr-ab1 (imatinib, nilotinib, dasatinib); Src (bosutinib) and Janus kinase 2 (lestaurtinib).

Non-limiting examples of chemotherapeutic agents include amsacrine, Trabectedin, retinoids (alitretinoin, tretinoin), arsenic trioxide, asparagine depleter asparaginase/pegaspargase), celecoxib, demecolcine, elesclomol, elsamitrucin, etoglucid, lonidamine, lucanthone, mitoguazone, mitotane, oblimersen, temsirolimus, and vorinostat.

Non-limiting examples of additional therapeutic molecules that can be linked to AFFIMER® polypeptides of the disclosure include flomoxef; fortimicin(s); gentamicin(s); glucosulfone solasulfone; gramicidin S; gramicidin(s); grepafloxacin; guamecycline; hetacillin; isepamicin; josamycin; kanamycin(s); flomoxef; fortimicin(s); gentamicin(s); glucosulfone solasulfone; gramicidin S; gramicidin(s); grepafloxacin; guamecycline; hetacillin; isepamicin; josamycin; kanamycin(s); bacitracin; bambermycin(s); biapenem; brodimoprim; butirosin; capreomycin; carbenicillin; carbomycin; carumonam; cefadroxil; cefamandole; cefatrizine; cefbuperazone; cefclidin; cefdinir; cefditoren; cefepime; cefetamet; cefixime; cefinenoxime; cefininox; cladribine; apalcillin; apicycline; apramycin; arbekacin; aspoxicillin; azidamfenicol; aztreonam; cefodizime; cefonicid; cefoperazone; ceforamide; cefotaxime; cefotetan; cefotiam; cefozopran; cefpimizole; cefpiramide; cefpirome; cefprozil; cefroxadine; cefteram; ceftibuten; cefuzonam; cephalexin; cephaloglycin; cephalosporin C; cephradine; chloramphenicol; chlortetracycline; clinafloxacin; clindamycin; clomocycline; colistin; cyclacillin; dapsone; demeclocycline; diathymosulfone; dibekacin; dihydrostreptomycin; 6-mercaptopurine; thioguanine; capecitabine; docetaxel; etoposide; gemcitabine; topotecan; vinorelbine; vincristine; vinblastine; teniposide; melphalan; methotrexate; 2-p-sulfanilyanilinoethanol; 4,4′-sulfinyldianiline; 4-sulfanilamidosalicylic acid; butorphanol; nalbuphine. streptozocin; doxorubicin; daunorubicin; plicamycin; idarubicin; mitomycin C; pentostatin; mitoxantrone; cytarabine; fludarabine phosphate; butorphanol; nalbuphine. streptozocin; doxorubicin; daunorubicin; plicamycin; idarubicin; mitomycin C; pentostatin; mitoxantrone; cytarabine; fludarabine phosphate; acediasulfone; acetosulfone; amikacin; amphotericin B; ampicillin; atorvastatin; enalapril; ranitidine; ciprofloxacin; pravastatin; clarithromycin; cyclosporin; famotidine; leuprolide; acyclovir; paclitaxel; azithromycin; lamivudine; budesonide; albuterol; indinavir; metformin; alendronate; nizatidine; zidovudine; carboplatin; metoprolol; amoxicillin; diclofenac; lisinopril; ceftriaxone; captopril; salmeterol; xinafoate; imipenem; cilastatin; benazepril; cefaclor; ceftazidime; morphine; dopamine; bialamicol; fluvastatin; phenamidine; podophyllinic acid 2-ethylhydrazine; acriflavine; chloroazodin; arsphenamine; amicarbilide; aminoquinuride; quinapril; oxymorphone; buprenorphine; floxuridine; dirithromycin; doxycycline; enoxacin; enviomycin; epicillin; erythromycin; leucomycin(s); lincomycin; lomefloxacin; lucensomycin; lymecycline; meclocycline; meropenem; methacycline; micronomicin; midecamycin(s); minocycline; moxalactam; mupirocin; nadifloxacin; natamycin; neomycin; netilmicin; norfloxacin; oleandomycin; oxytetracycline; p-sulfanilylbenzylamine; panipenem; paromomycin; pazufloxacin; penicillin N; pipacycline; pipemidic acid; polymyxin; primycin; quinacillin; ribostamycin; rifamide; rifampin; rifamycin SV; rifapentine; rifaximin; ristocetin; ritipenem; rokitamycin; rolitetracycline; rosaramycin; roxithromycin; salazosulfadimidine; sancycline; sisomicin; sparfloxacin; spectinomycin; spiramycin; streptomycin; succisulfone; sulfachrysoidine; sulfaloxic acid; sulfamidochrysoidine; sulfanilic acid; sulfoxone; teicoplanin; temafloxacin; temocillin; tetroxoprim; thiamphenicol; thiazolsulfone; thiostrepton; ticarcillin; tigemonam; tobramycin; tosufloxacin; trimethoprim; trospectomycin; trovafloxacin; tuberactinomycin; vancomycin; azaserine; candicidin(s); chlorphenesin; dermostatin(s); filipin; fungichromin; mepartricin; nystatin; oligomycin(s); perimycin A; tubercidin; 6-azauridine; 6-diazo-5-oxo-L-norleucine; aclacinomycin(s); ancitabine; anthramycin; azacitadine; azaserine; bleomycin(s); ethyl biscoumacetate; ethylidene dicoumarol; iloprost; lamifiban; taprostene; tioclomarol; tirofiban; amiprilose; bucillamine; gusperimus; gentisic acid; glucamethacin; glycol salicylate; meclofenamic acid; mefenamic acid; mesalamine; niflumic acid; olsalazine; oxaceprol; S-enosylmethionine; salicylic acid; salsalate; sulfasalazine; tolfenamic acid; carubicin; carzinophillin A; chlorozotocin; chromomycin(s); denopterin; doxifluridine; edatrexate; eflornithine; elliptinium; enocitabine; epirubicin; mannomustine; menogaril; mitobronitol; mitolactol; mopidamol; mycophenolic acid; nogalamycin; olivomycin(s); peplomycin; pirarubicin; piritrexim; prednimustine; procarbazine; pteropterin; puromycin; ranimustine; streptonigrin; thiamiprine; mycophenolic acid; procodazole; romurtide; sirolimus (rapamycin); tacrolimus; butethamine; fenalcomine; hydroxytetracaine; naepaine; orthocaine; piridocaine; salicyl alcohol; 3-amino-4-hydroxybutyric acid; aceclofenac; alminoprofen; amfenac; bromfenac; bromosaligenin; bumadizon; carprofen; diclofenac; diflunisal; ditazol; enfenamic acid; etodolac; etofenamate; fendosal; fepradinol; flufenamic acid; Tomudex (N-[[5-[[(1,4-Dihydro-2-methyl-4-oxo-6-quinazolinyl)methyl]methylamino]-2-thienyl]carbonyl]-L-glutamic acid), trimetrexate, tubercidin, ubenimex, vindesine, zorubicin; argatroban; coumetarol and dicoumarol.

Non-limiting examples of cytotoxic factors include diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, Momordica charantia inhibitor, curcin, crotin, Saponaria officinalis inhibitor, mitogellin, restrictocin, phenomycin, and enomycin.

Non-limiting examples of neurotransmitters include arginine, aspartate, glutamate, gamma-aminobutyric acid, glycine, D-serine, acetylcholine, dopamine, norepinephrine (noradrenaline), epinephrine (adrenaline), serotonin (5-hydroxytryptamine), histamine, phenethylamine, N-methylphenethylamine, tyramine, octopamine, synephrine, tryptamine, N-methyltryptamine, anandamide, 2-arachidonoylglycerol, 2-arachidonyl glyceryl ether, N-arachidonoyl dopamine, virodhamine, adenosine, adenosine triphosphate, bradykinin, corticotropin-releasing hormone, urocortin, galanin, galanin-like peptide, gastrin, cholecystokinin, adrenocorticotropic hormone, proopiomelanocortin, melanocyte-stimulating hormones, vasopressin, oxytocin, Neurophysin I, Neurophysin II, Neuromedin U, Neuropeptide B, Neuropeptide S, Neuropeptide Y, Pancreatic polypeptide, Peptide YY, enkephalin, dynorphin, endorphin, endomorphin, nociceptin/orphanin FQ, Orexin A, Orexin B, kisspeptin, Neuropeptide FF, prolactin-releasing peptide, pyroglutamylated rfamide peptide, secretin, motilin, glucagon, glucagon-like peptide-1, glucagon-like peptide-2, vasoactive intestinal peptide, growth hormone-releasing hormone, pituitary adenylate cyclase-activating peptide, somatostatin, Neurokinin A, Neurokinin B, Substance P, Neuropeptide K, agouti-related peptide, N-acetylaspartylglutamate, cocaine- and amphetamine-regulated transcript, bombesin, gastrin releasing peptide, gonadotropin-releasing hormone, melanin-concentrating hormone, nitric oxide, carbon monoxide, and hydrogen sulfide.

Non-limiting examples of metabolic hormones, such as incretins (which stimulate a decrease in blood glucose levels), include glucagon-like peptide-1 (GLP-1) and gastric inhibitory peptide (GIP) and anologs thereof, such as dulaglutide (TRULICITY®), exenatide (BYETTA®), liraglutide (VICTOZA®), and exenatide extended-release (BYDUREON®).

### Polynucleotides

A polynucleotide (also referred to as a nucleic acid) is a polymer of nucleotides of any length, and may include deoxyribonucleotides, ribonucleotides, modified nucleotides or bases, and/or their analogs, or any substrate that can be incorporated into a polymer by DNA or RNA polymerase. In some embodiments, a polynucleotide herein encodes a polypeptide, such as an anti-HSA AFFIMER® polypeptide. As known in the art, the order of deoxyribonucleotides in a polynucleotide determines the order of amino acids along the encoded polypeptide (e.g., protein).

A polynucleotide sequence may be any sequence of deoxyribonucleotides and/or ribonucleotides, may be single-stranded, double-stranded, or partially double-stranded. The length of a polynucleotide may vary and is not limited. Thus, a polynucleotide may comprise, for example, 2 to 1,000,000 nucleotides. In some embodiments, a polynucleotide has a length of 100 to 100,000, a length of 100 to 10,000, a length of 100 to 1,000, a length of 100 to 500, a length of 200 to 100,000, a length of 200 to 10,000, a length of 200 to 1,000, or a length of 200 to 500 nucleotides.

A vector herein refers to a vehicle for delivering a molecule to a cell. In some embodiments, a vector is an expression vector comprising a promoter (e.g., inducible or constitutive) operably linked to a polynucleotide sequence encoding a polypeptide. Non-limiting examples of vectors include viral vectors (e.g., adenoviral vectors, adeno-associated virus vectors, and retroviral vectors), naked DNA or RNA expression vectors, plasmids, cosmids, phage vectors, DNA and/or RNA expression vectors associated with cationic condensing agents, and DNA and/or RNA expression vectors encapsulated in liposomes. Vectors may be transfected into a cell, for example, using any transfection method, including, for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics technology (biolistics).

### EXAMPLES

### Example 1. Human Serum Albumin (HSA) and Mouse Serum Albumin (MSA) AFFIMER® Binder Selection

Selection of HSA or MSA binding phage from the AFFIMER® library was carried out using approximately 1×10¹² phage added from a library of size approximately 6×10¹⁰ diversity. The HSA binding peptides of the disclosure were identified by selection from the phage display library comprising random loop sequences nine amino acids in length displayed in a constant AFFIMER® framework backbone based upon the sequence for SQT. Suspensions of phage were incubated with target antigen (either biotinylated antigen captured on streptavidin beads or unbiotinylated antigen captured on a plate). Unbound phage were then washed away and, subsequently, bound phage were eluted by incubating the antigen with low pH, followed by high pH. Then, E. coli were infected with released, pH neutralized phage and a preparation of first round phage was obtained. The cycle was repeated two or three times. In order to enrich for targeting phage, the stringency conditions were increased in the later rounds of selection. Increased stringency conditions included increasing the number of wash steps, reducing the antigen concentration, and/or preselecting with blocked streptavidin beads or wells coated with blocking reagent.

Antigens used herein for phage selections were HSA (Sigma; A3782) and MSA (Alpha Diagnostics; ALB13-N-25). Antigen biotinylation was carried out in-house using the EZ Link Sulfo-NHS-LC Biotin kit (Pierce).

Following selection by successive rounds of phage amplification, HSA and MSA binding clones were identified by a phage ELISA as described below. Following phage selections, individual bacterial clones containing the phagemid vector were moved from titration plates into 96 well cell culture format. Recombinant phage particles that displayed HSA AFFIMER® polypeptide fused to the gene-III minor coat protein were released into the culture supernatant following helper phage rescue and overnight growth. The phage contained in the supernatants were subsequently screened for binding to antigen by ELISA. Phage-displaying AFFIMER® binding to antigen immobilized on a plate was detected with an HRP-conjugated anti-M13 monoclonal antibody (GE Healthcare), and the ELISA was developed using 1-step Ultra TMB-ELISA substrate (Thermo Scientific).

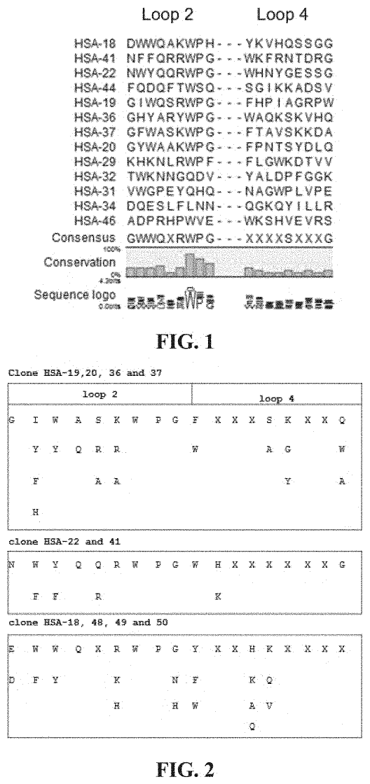

An alignment of AFFIMER® polypeptide loop 2 and loop 4 identified from the phage selections was performed (FIG. 1). From the alignment, families of sequence motifs from the HSA and MSA phage selections were identified (FIG. 2).

### Example 2. AFFIMER® Polypeptide E. coli Protein Production

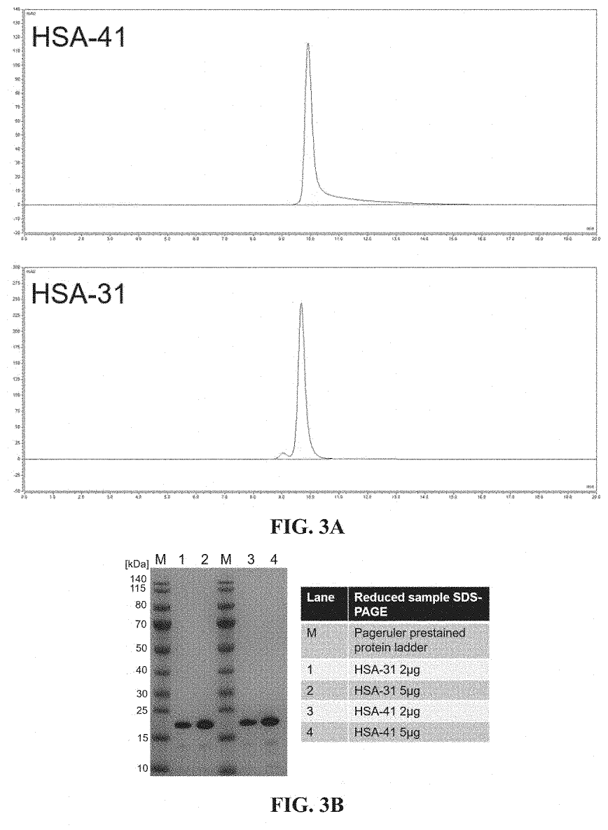

All AFFIMER® polypeptides expressed in E. coli have been cloned with a C-terminal hexa-HIS tag (HHHHHH; SEQ ID NO: 168) to simplify protein purification with immobilized metal affinity chromatography resin (IMAC resin). When required, additional peptide sequences can be added between the AFFIMER® polypeptide and the HIS tag such as MYC (EQKLISEEDL; SEQ ID NO: 162) for detection or a TEV protease cleavage site (ENLYFQ(G/S); SEQ ID NO: 163) to allow for the removal of tags. AFFIMER® proteins were expressed from E. coli and purified using IMAC, IEX, and SEC. AFFIMER® monomer purification from E. coli was performed by transforming the expression plasmid pD861 (Atum) into BL21 E. coli cells (Millipore) using the manufacturer's protocol. The total transformed cell mixture was plated onto LB agar plates containing 50 μg/mlkanamycin (AppliChem) and incubated at 37° C. overnight. The following day, the lawn of transformed E. coli was transferred to a sterile flask of 1× terrific broth media (Melford) and 50 μg/mlkanamycin and incubated at 30° C. shaking at 250 rpm. Expression was induced with 10 mM rhamnose (Alfa Aesar) once the cells reached an optical density OD₆₀₀ of approximate 0.8-1.0. The culture was then incubated for a further 5 hours at 37° C. Cells were harvested by centrifuging and lysing the resulting cell pellet. AFFIMER® polypeptide purification was performed using batch bind affinity purification of His-tagged protein. Specifically, nickel agarose affinity resin (Super-NiNTA500; Generon) was used. The resin was washed with NPI20 buffer (50 mM sodium phosphate, 0.5M NaCl, 20 mM imidazole) and the bound protein was eluted with 5 column volumes (CV) of NPI400 buffer. Eluted protein was then purified by cation exchange using an CM FF ion exchange column (GE) in running buffer 20 mM sodium acetate pH 5.2 for clone HSA-31 (SEQ ID NO: 113) and 25 mM MES pH 6.0 for clone HSA-41 (SEQ ID NO: 116). Both protein purifications further included a 0.1% triton 114× (Sigma) wash step and the protein was eluted with a 1M NaCl linear gradient. A third stage purification was performed on a preparative SEC performed using the HiLoad 26/600 Superdex 75 pg (GE Healthcare) run in PBS 1× buffer. Expression and purity of clone HSA-41 (SEQ ID NO: 116) and HSA-31 (SEQ ID NO: 113) was analysed using SEC-HPLC (FIG. 3A) with an Acclaim SEC-300 column (Thermo) using a PBS 1× mobile phase. The protein yield was estimated using Nanodrop (Thermo) A280 readings and the final product was run on an SDS-PAGE Bolt Bis Tris plus 4-12% gel (Thermo) in Novex™ 20X Bolt™ MES SDS running buffer (Thermo) at 200 volts, with samples heated in reducing buffer. Protein bands on the gel were stained with Quick Commassie

(Generon). PageRuler prestained protein molecular weight marker (Thermo) was run on the gel to estimate the molecular weight of the fusion proteins (FIG. 3B) following the three-stage purification.

### Example 3. Serum Albumin Binding AFFIMER® Polypeptide Characterization

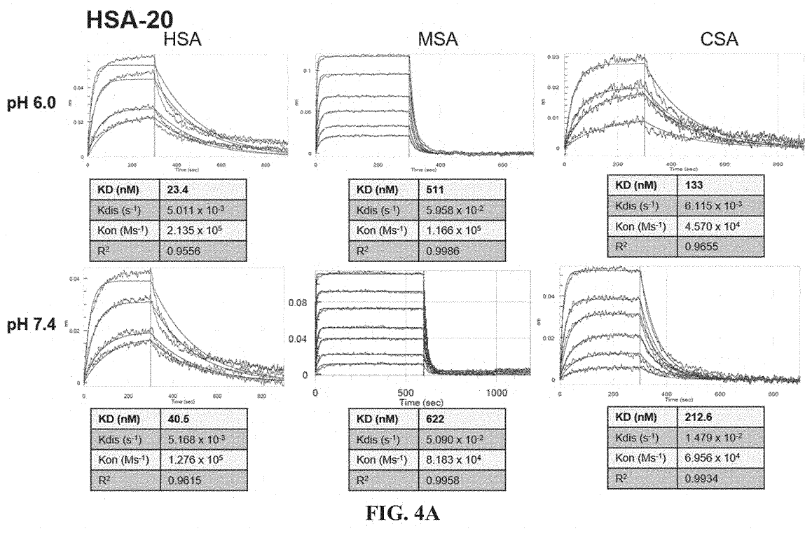

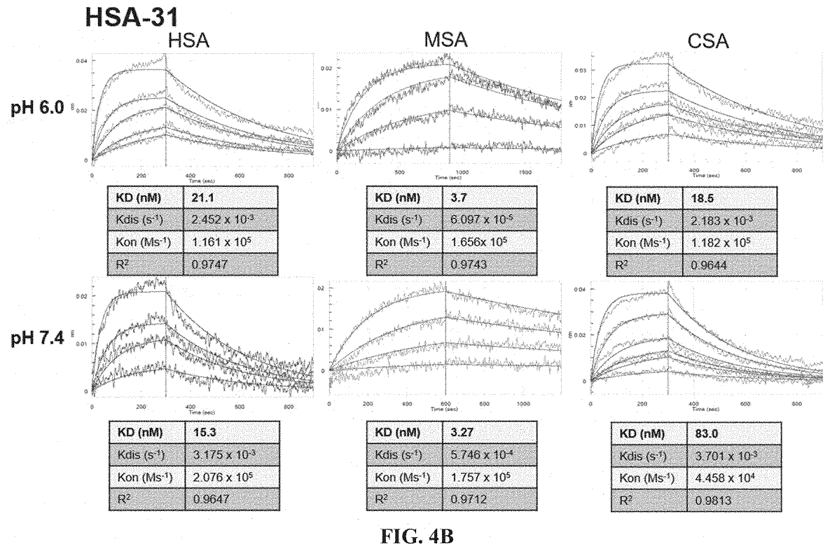

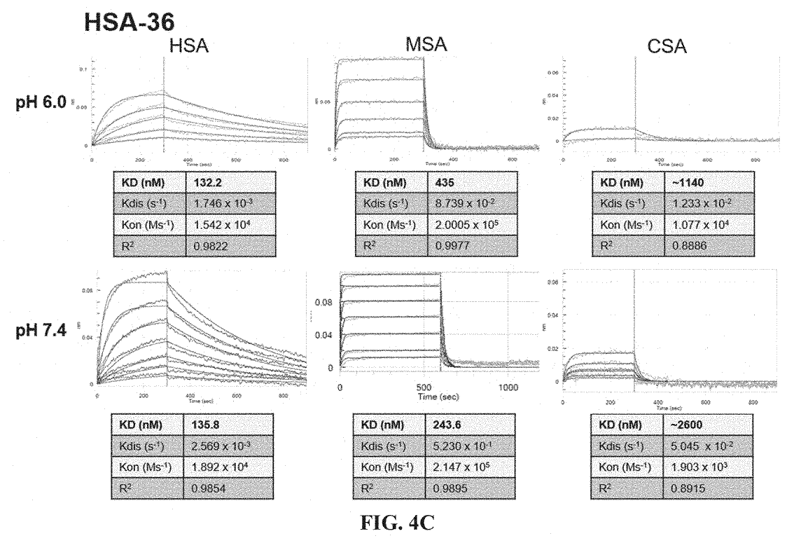

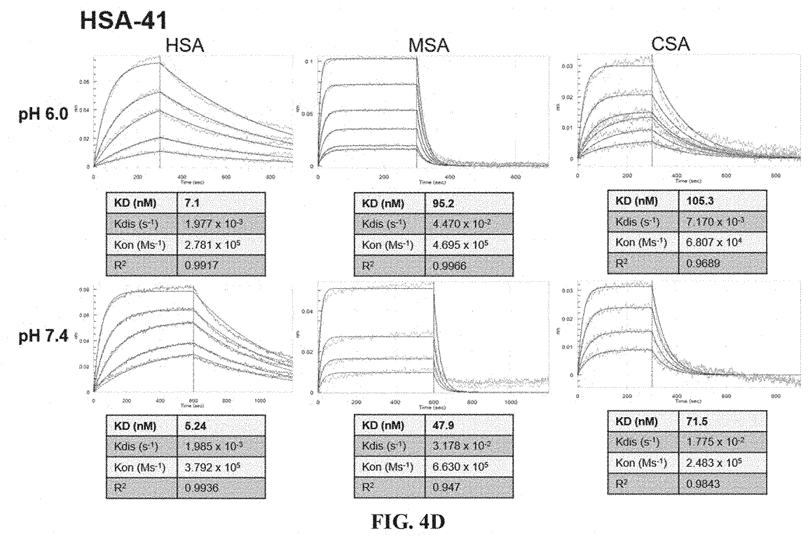

Binding affinities of purified HSA-20 (SEQ ID NO: 111), HSA-31 (SEQ ID NO: 113), HSA-36 (SEQ ID NO: 114), HSA-41 (SEQ ID NO: 116) AFFIMER® proteins to human, mouse, and cynomolgus sera were assessed by Biolayer Interferometry (Octet) at both pH 6.0 and pH 7.4. HSA binding affinities ranged from 7.1 nM to 135.5 nM at pH 6.0, MSA affinities ranged from 3.7 nM to 833.7 nM at pH 6.0, and CSA affinities ranged from 18.5 nM to 1.15 uM at pH 6.0 (data shown in FIGS. 4A-4D, Table 4). Biotinylated antigen was captured onto SA sensors at 1 μg/ml for 600 seconds in a buffer comprising PBS-T (0.01% Tween 20)+1% casein at either pH 6.0 or 7.4. Association was carried out for 300 seconds and dissociation for 600 seconds, and regeneration was performed using 10 mM glycine pH 1.5 (GE Healthcare) for 3×5 seconds. All steps were carried out at 1000 rpm and 25° C. Purified AFFIMER® polypeptide in two-fold serial dilutions was analyzed at a starting concentration of approximately 10× K_{D value}. Kinetics analyses were carried out using the Octet data analysis software, subtracting the reference sensor (loaded with antigen), aligning the Y-axis to baseline, and using inter-step correction to align association to dissociation. Savitzky-Golay filtering was applied and the data processed. Analysis of the data was carried out with a 1:1 model, global fit, Rₘₐₓ unlinked by sensor.

TABLE 4
K_{D} values for AFFIMER ® proteins binding to human, mouse
and cynomolgus serum albumin protein at pH 6.0 and 7.4.

| | MSA K_{D} nM | | CSA K_{D} nM | | HSA K_{D} nM | |
|---|---|---|---|---|---|---|
| Clone | pH 6.0 | pH 7.4 | pH 6.0 | pH 7.4 | pH 6.0 | pH 7.4 |
| HSA-18 | 618.2 | 981.1 | 68.7 | 106.5 | 17.9 | 36.1 |
| (SEQ ID NO: 110) | | | | | | |
| HSA-20 | 511.4 | 622.3 | 133 | 212.6 | 23.4 | 40.5 |
| (SEQ ID NO: 111) | | | | | | |
| HSA-31 | 3.7 | 3.3 | 18.5 | 83 | 21.1 | 15.3 |
| (SEQ ID NO: 113) | | | | | | |
| HSA-36 | 435 | 243.6 | 1140 | 2600 | 132.2 | 135.8 |
| (SEQ ID NO: 114) | | | | | | |
| HSA-41 | 95.2 | 47.9 | 105.3 | 71.5 | 7.1 | 5.2 |
| (SEQ ID NO: 116) | | | | | | |
| HSA-22 | 334 | — | 146 | 347.9 | 44.7 | 152.3 |
| (SEQ ID NO: 112) | | | | | | |
| HSA-37 | 833.7 | — | 231.3 | 221.4 | 135.5 | 113.1 |
| (SEQ ID NO: 115) | | | | | | |

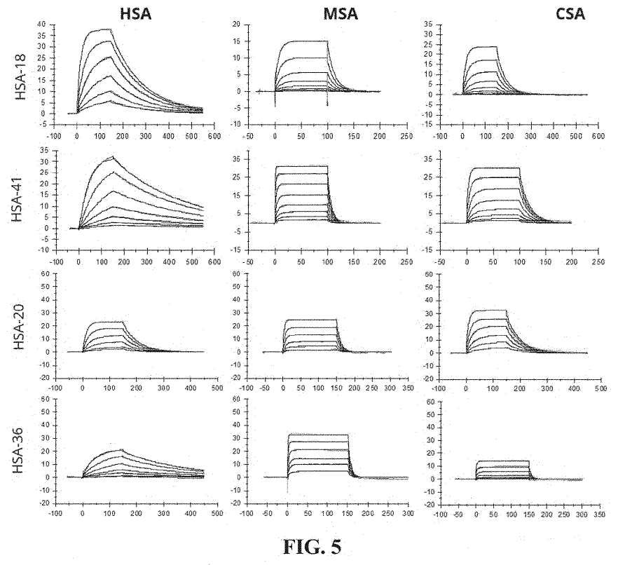

Affinities for mouse, human, and cynomolgus serum were also measured at pH 7.4 for four (4) AFFIMER® binders using surface plasmon resonance (SPR) (FIG. 5, Table 5). Biacore T200 kinetic analysis was performed using running buffer HBS-EP+ (GE Healthcare) and series S sensor CM5 chip (GE Healthcare) immobilized on surface Fc2, Fc3, and Fc4 with HSA (Sigma; A37812), MSA (Sigma; A3559), or cyno serum albumin (CSA) (Abcam; Ab184894), respectively, in 10 mM sodium acetate pH 5.0 (GE Healthcare) using amine coupling reagents (GE Healthcare). A concentration titration of AFFIMER® monomers was run as analyte at a flow rate of 30 μl/min or 60 μl/min. Fc2-1, Fc3-1 and Fc4-1 kinetic data was blank subtracted and fit to a 1:1 Langmuir binding model (BIAcore Evalution software; GE) to calculate K_{D} values.

TABLE 5
Binding Kinetics to human, mouse and cynomolgus
serum albumin of four AFFIMER ® binders
measured using Biacore SPR measured at pH 7.4

| AFFIMER ® | | | | | |
|---|---|---|---|---|---|
| Binder | Ligand | ka [1/Ms] | kd [1/s] | KD [M] | Chi² |
| HSA-18 | HSA | 5.41E+04 | 7.89E−03 | 1.46E−07 | 0.0771 |
| (SEQ ID NO: | MSA | 3.41E+04 | 7.42E−02 | 2.18E−06 | 0.0966 |
| 110) | CSA | 4.83E+04 | 2.61E−02 | 5.42E−07 | 0.148 |
| HSA-41 | HSA | 1.43E+06 | 3.70E−03 | 2.59E−09 | 0.0655 |
| (SEQ ID NO: | MSA | 2.04E+06 | 1.39E−01 | 6.78E−08 | 0.236 |
| 116) | CSA | 4.88E+05 | 5.46E−02 | 1.12E−07 | 0.236 |
| HSA-20 | HSA | 5.22E+04 | 1.83E−02 | 3.50E−07 | 0.0503 |
| (SEQ ID NO: | MSA | 1.15E+05 | 9.81E−02 | 8.55E−07 | 0.344 |
| 111) | CSA | 4.76E+04 | 1.99E−02 | 4.19E−07 | 0.372 |
| HSA-36 | HSA | 2.25E+04 | 4.51E−03 | 2.00E−07 | 0.0912 |
| (SEQ ID NO: | MSA | 7.26E+05 | 1.50E−01 | 2.07E−07 | 0.481 |
| 114) | CSA | 1.06E+05 | 1.60E−01 | 1.51E−06 | 0.289 |

### Example 4. AFFIMER® Serum Albumin Species Cross Reactivity

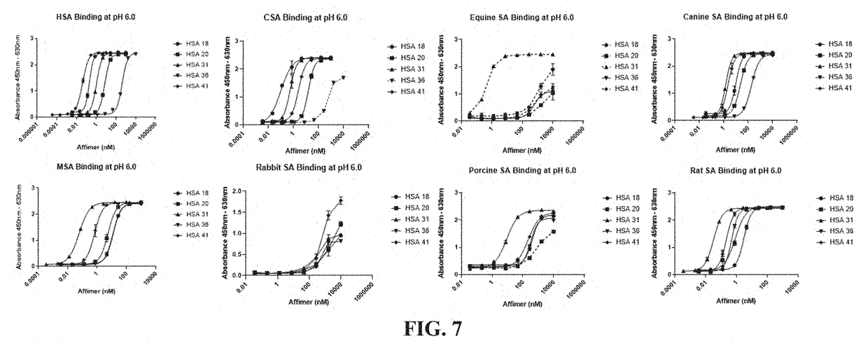

Five (5) AFFIMER® proteins with the highest affinity for HSA were analyzed for cross reactivity to human, cynomolgus, equine, canine, mouse, rabbit, porcine, and rat serum albumin by binding ELISA at both pH 6.0 and pH 7.4 (FIGS. 7A-7B, Table 6). Briefly, ELISA was performed as follow:

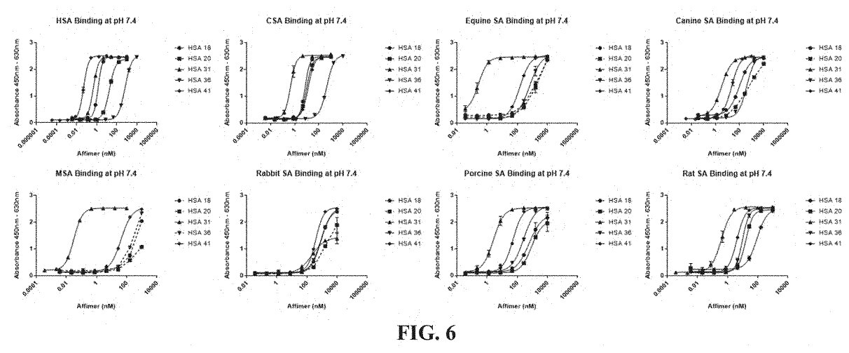

Human (Sigma-Aldrich); Cynomolgus and Mouse (Abcam); Equine (Abcam); Canine (Abcam); Mouse (Sigma-Aldrich); Rabbit (Sigma-Aldrich); Porcine (Sigma-Aldrich);, and Rat (Sigma-Aldrich) serum albumin were coated overnight on 96 well plates (Corning Costar) at 1 μg/ml in PBS at the appropriate pH (pH 6.0 or 7.4). Plates were saturated using 5% Casein (Sigma-Aldrich) buffer in PBS. Following blocking, a dilution of the five (5) lead binders (HSA-18 (SEQ ID NO:110), HSA-20 (SEQ ID NO:111), HSA-36 (SEQ ID NO:114), HSA-31 (SEQ ID NO:113) and HSA-41 (SEQ ID NO:116)) were added to the plates and incubated for 90 minutes. Plates were then washed and AFFIMER® proteins were detected using a biotinylated polyclonal antibody anti-Cystatin (R&D System) for 90 minutes. After washing, streptavidin-HRP (Thermo Fisher Scientific) was added and incubated for 30 minutes. After a final wash, development of the reaction was performed using TMB (Thermo Fisher Scientific) and plates were read using a plate reader at 450 nm (FIG. 6).

TABLE 6
Serum Albumin Species Cross-Reactivity Binding at pH 7.4 - EC₅₀ (nM)

| Species | HSA-18 | HSA-20 | HSA-31 | HSA-36 | HSA-41 |
|---|---|---|---|---|---|
| Human | 1.41 | 18.90 | 0.47 | 687.3 | 0.05 |
| Cynomolgus | 7.14 | 9.55 | 0.43 | 447.1 | 12.85 |
| Mouse | NA | NA | 0.03 | NA | 42.5 |
| Rabbit | 847.1 | NA | 210.9 | 863.0 | 380.9 |
| Equine | NA | NA | 0.25 | 661.8 | 150.1 |
| Canine | 96.47 | NA | 3.17 | 383.4 | 21.49 |
| Porcine | 615.0 | 893.8 | 2.60 | 267.5 | 51.71 |
| Rat | 89.43 | 16.10 | 0.39 | 9.73 | 4.18 |

### Example 5. Pharmacokinetic Profile of Five Lead Serum Albumin Binders in Mouse

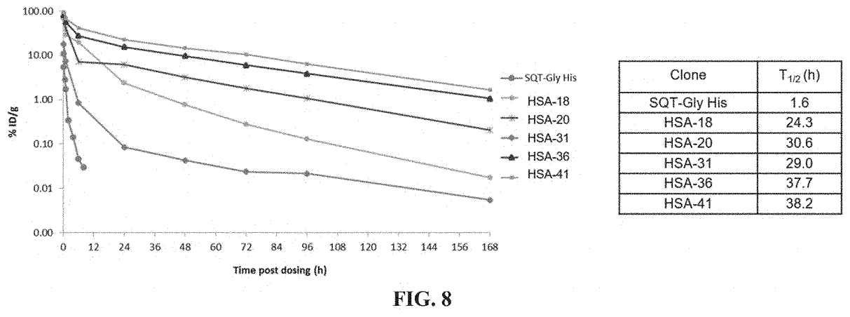

Five (5) AFFIMER® proteins, HSA-18 (SEQ ID NO: 110), HSA-20 (SEQ ID NO: 111), HSA-31(SEQ ID NO: 113), HSA-36 (SEQ ID NO: 114), HSA-41 (SEQ ID NO: 116), with a range of different affinities and association and dissociation constants for MSA were selected to be tested in vivo. AFFIMER® proteins were radiolabeled using 1-125 and dosed at 10 mg/kg as a bolus IV injection to three (3) mice per time point. The serum concentration of AFFIMER® proteins was determined for eight (8) time points (between 0.25-168 hours) over seven (7) days by measurement of radioactivity (FIG. 8, Table 7). All AFFIMER® proteins had an increased half-life compared to the SQT gly His control monomer AFFIMER® protein, and all clones were well-tolerated in vivo.

TABLE 7
Pharmacokinetic parameters in mouse of the AFFIMER ® proteins:
(half-life, t_{1/2}) and exposure (AUC 0-t) in a non-compartmental analysis

| Clone | T1/2 (hours) | AUC 0-t (h * mg/mL) |
|---|---|---|
| HSA-41 | 38.2 | 5670 |
| HSA-36 | 37.7 | 3435 |
| HSA-20 | 30.6 | 1401 |
| HSA-18 | 24.3 | 1059 |
| HSA-31 | 29.0 | 112 |
| SQT-Gly His (control) | 1.6 | 18.1 |

### Example 6. Pharmacokinetics Profile of C-Terminal His Tag Cleaved Half-Life Extended AFFIMER® Polypeptides in Mouse

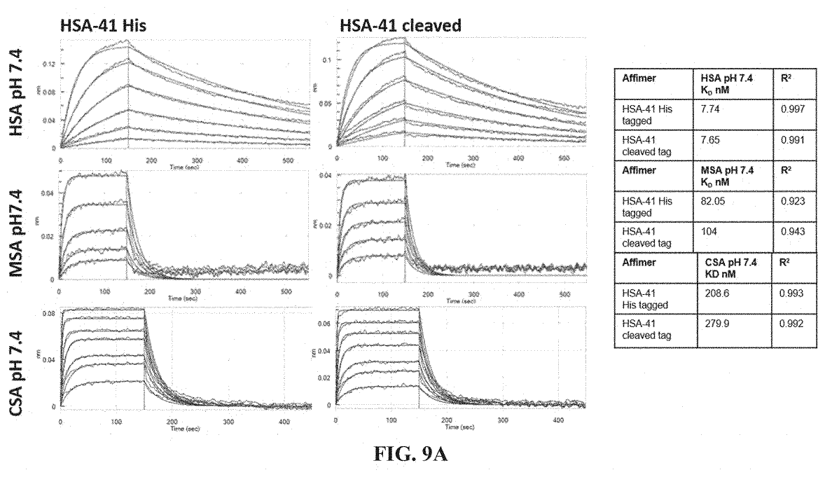

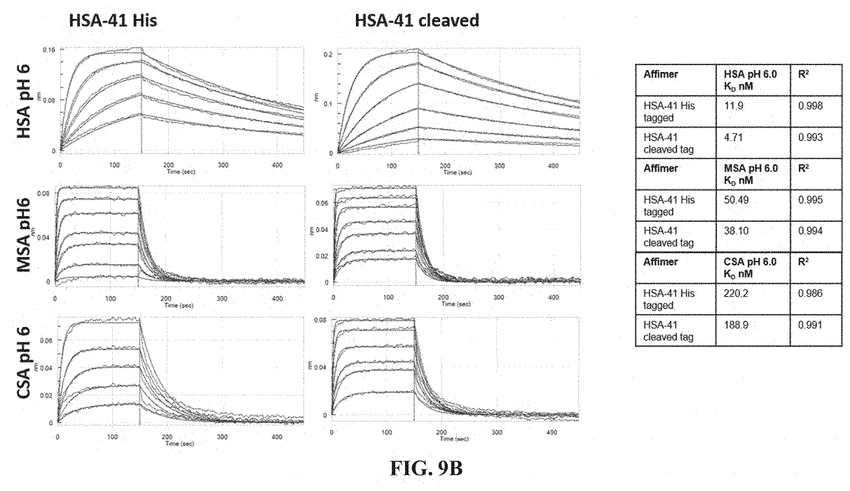

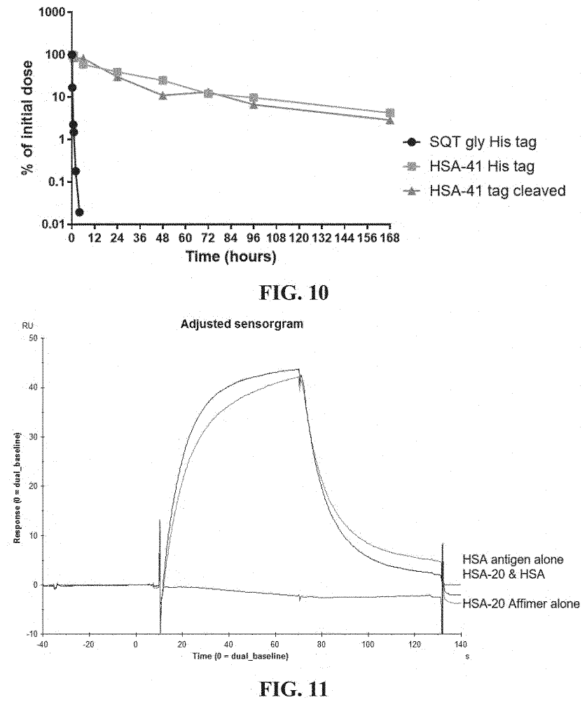

To ensure the presence of the C-terminal 6x His tag did not influence the pharmacokinetic profile of the AFFIMER® polypeptides in vivo, a TEV cleavable linker was introduced between the protein and the purification tag. Cleavage of the C-terminal 6x His tag of lead clone HSA-41 (SEQ ID NO: 116) was possible due to the inclusion of a TEV cleavage site, amino acid sequence ENLYFQG (SEQ ID NO: 164), following the AFFIMER® polypeptide-Myc C-terminus gene insert. AFFIMER® polypeptides were incubated with AcTEV for 1 hour at 30° C. as recommend by the manufacturer (Invitrogen) and removal of cleaved His tag by binding NiNTA resin (Generon) and collecting the cleaved protein flow through. An anti-His (R&D Systems) Western blot was performed to confirm the His Tag could no longer be detected. AFFIMER® polypeptide binding kinetics were measured using the Octet as described in Example 3 and show the AFFIMER® polypeptides retain binding properties to HSA and MSA at pH 7.4 following cleavage of the C-terminal 6x His tag (FIG. 9). Pharmacokinetic profile of IV-injected His tag cleaved AFFIMER® protein compared to C-terminally His-tagged AFFIMER® protein was evaluated. Eight (8) time points were analyzed using an anti-cystatin sandwich ELISA on pooled serum from three (3) mice per timepoint, results show the proteins have a similar extended PK with and without the presence of a C-terminal His tag (FIG. 10).

### Example 7. FcRn Binding of HSA in the Presence of Serum Albumin Binding AFFIMER® Polypeptide

Example of the binding of HSA to recombinant FcRn is not affected when 500 nM AFFIMER® polypeptide was pre-incubated with HSA at pH 6.0 (FIG. 11). FcRn Biacore binding analysis was performed using Biotin CAPture chip according to the manufacturer's protocol (GE Healthcare). Briefly, Biotin CAPture reagent was run for 300 sec at a 2 μl/min flow rate over surfaces Fc1 and Fc2 followed by the capture of biotinylated FcRn (Amsbio) at 10 μg/mL with a contact time of 50 seconds, run at 10 μl/min delivered to Fc2 only. A constant HSA concentration of 2.5 μM was flowed over surfaces Fc1 and Fc2 with and without being pre-mixed with HSA-20 (SEQ ID NO: 111) at 500 nM in PBS-T pH 6.0. No difference was seen in the binding of HSA to FcRn when the AFFIMER® polypeptide was bound to HSA compared to albumin alone. Surface Fc1 and Fc2 regeneration was performed according to the manufacturer's protocol.

### Example 8. PD-L1 Binding AFFIMER® Half-Life Extended In-Line Fusion (ILF) Dimers

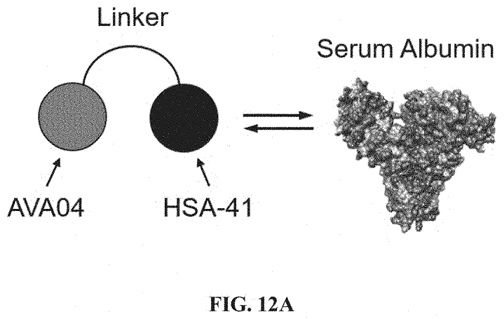

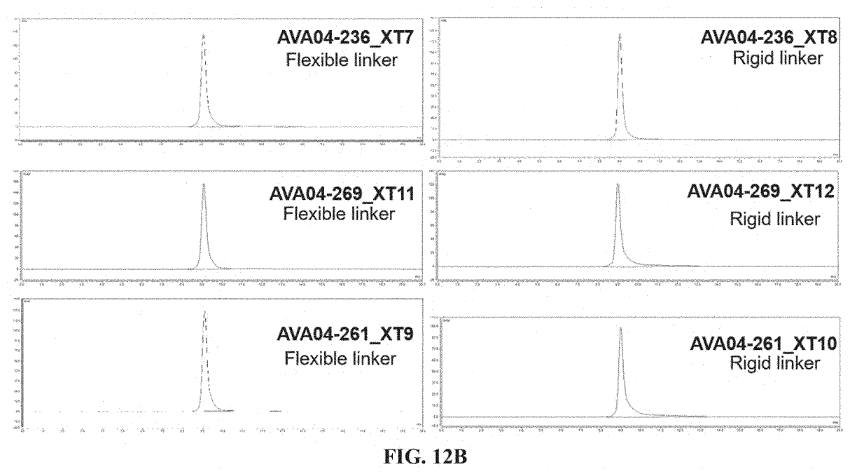

The half-life of three (3) PD-L1 binding AFFIMER® polypeptides, AVA04-236 (SEQ ID NO: 117 and 118), AVA04-261 (SEQ ID NO: 119 and 120), and AVA04-269 (SEQ ID NO: 121 and 122), was extended by genetically fusing to HSA-41 (SEQ ID NO: 116) at the N- or C-terminus. A schematic representation of half-life extended PD-L1 AFFIMER® polypeptides formatted as dimer genetic fusions using a rigid A(EAAAK)₆ (SEQ ID NO: 161) or flexible (G45)₆ (SEQ ID NO: 165) repetitive genetic linkers is give in FIG. 12A, and a table of ILF orientation and nomenclature is shown in Table 8. ILF dimer production from E. coli was performed as described in Example 3. Briefly, protein was purified using three stages: affinity capture, IEX, and preparative SEC. Final ILF protein purity was assessed using SEC-HPLC and shown to be >95% pure (FIG. 12B). Biacore kinetic analysis showed both AFFIMER® polypeptides genetically fused were able to engage target antigens, human PD-L1-Fc (R&D Systems), and HSA (Sigma) (Table 9). Biacore was performed as is described in Example 3 to analyze HSA binding. To analyze PD-L1-Fc binding kinetics, a Biacore T200 kinetic analysis was performed using running buffer HBS-EP+ and series S sensor CM5 (GE Healthcare) chip Fc2 immobilized with PD-L1-Fc (R&D Systems) in 10 mM sodium acetate pH 4.0 using amine coupling reagents (GE Healthcare). The concentration titration of ILF AFFIMER® polypeptides was run as analyte at a flow rate of 30 μl/min. Regenerated PD-L1-Fc was immobilized on a surface with 3 mM NaOH (GE healthcare) for 20 seconds at 20 μl/min flow rate. The Fc2-1 data blank was subtracted and fit to a 1:1 Langmuir binding model (Biacore evaluation software; GE) to calculate an apparent K_{D} value.

TABLE 8
Nomenclature of PD-L1 (AVA04) binding AFFIMER ® ILF proteins
half-life extended with HSA binding AFFIMER ® proteins

| | AVA04 | | Linker | AF- | SEQ |
|---|---|---|---|---|---|
| AFFIMER ® | AF- | | SEQ | FIMER ® | ID |
| Format | FIMER ® | Linker | ID NO: | XT | NO. |
| AVA04-236 XT7 | AVA04-236 | (G4S)₆ | 165 | HSA-41 | 117 |
| AVA04-236 XT8 | AVA04-236 | A(EAAAK)₆ | 161 | HSA-41 | 118 |
| AVA04-261 XT9 | AVA04-261 | (G4S)₆ | 165 | HSA-41 | 119 |
| AVA04-261 XT10 | AVA04-261 | A(EAAAK)₆ | 161 | HSA-41 | 120 |
| AVA04-269 XT11 | AVA04-269 | (G4S)₆ | 165 | HSA-41 | 121 |
| AVA04-269 XT12 | AVA04-269 | A(EAAAK)₆ | 161 | HSA-41 | 122 |

TABLE 9
BIACORE ™ Kinetic Analysis of AFFIMER ® Proteins

| | Dimer | HuSA kinetics | | | PD-L1 Kinetics | | |
|---|---|---|---|---|---|---|---|
| Protein | linker | ka (1/Ms) | kd (1/s) | KD (nM) | ka (1/Ms) | kd (1/s) | KD (nM) |
| HSA-41 | | 1.48E+06 | 3.54E−03 | 2.4 | | | |
| AVA04-236 | | | | | 1.80E+06 | 9.77E−03 | 5.43 |
| AVA04-236 XT7 | Flexible | 2.53E+05 | 3.36E−03 | 13.3 | 1.57E+06 | 7.63E−03 | 4.86 |
| AVA04-236 XT8 | rigid | 2.67E+05 | 2.52E−03 | 9.5 | 1.52E+06 | 6.95E−03 | 4.57 |
| AVA04-261 | | | | | 3.68E+06 | 1.09E−02 | 2.95 |
| AVA04-261 XT9 | Flexible | 2.69E+05 | 3.57E−03 | 13.3 | 3.95E+05 | 7.07E−03 | 17.9 |
| AVA04-261 XT10 | rigid | 2.99E+05 | 2.91E−03 | 9.7 | 8.75E+05 | 8.26E−03 | 9.44 |
| AVA04-269 | | | | | 2.05E+06 | 3.45E−03 | 1.68 |
| AVA04-269 XT11 | Flexible | 1.51E+05 | 2.34E−03 | 15.5 | 7.96E+05 | 3.54E−03 | 4.45 |
| AVA04-269 XT12 | rigid | 1.77E+05 | 2.28E−03 | 12.9 | 1.14E+06 | 4.31E−03 | 3.8 |

### Example 9. PD-L1 Binding AFFIMER® Dimers Half-Life Extended in Line Fusions Trimers

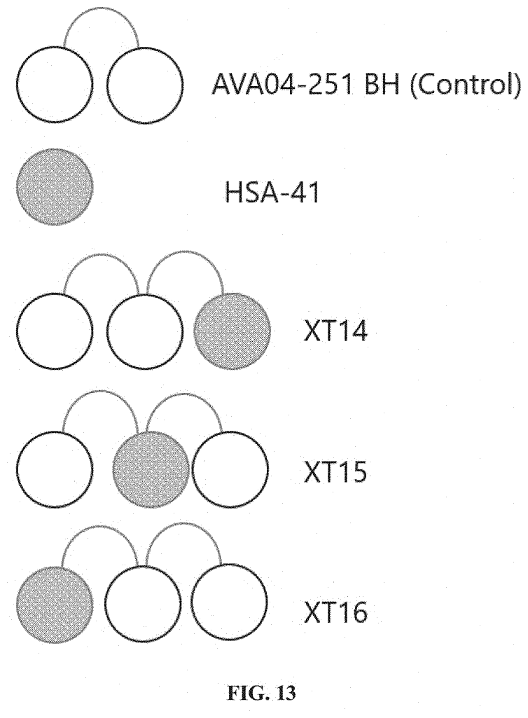

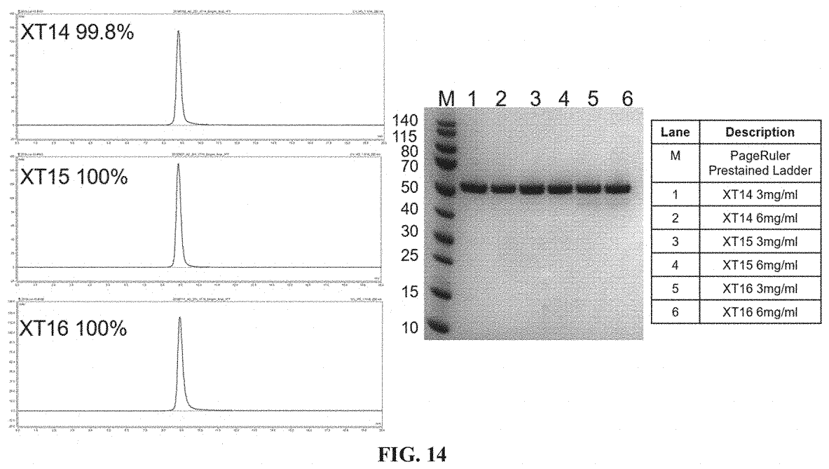

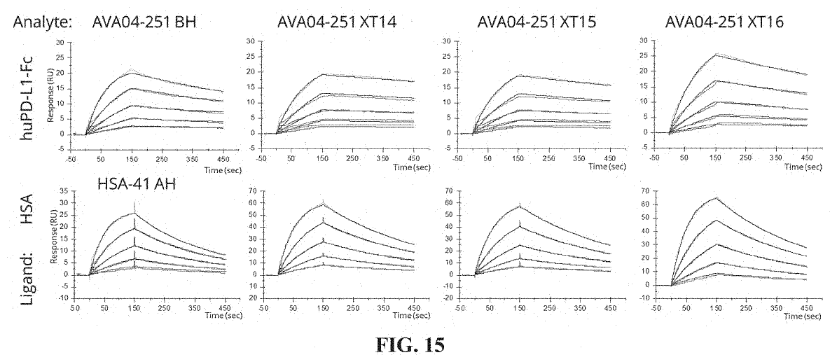

FIG. 13 shows a schematic representation of PD-L1 binding AFFIMER® dimer (two monomers of SEQ ID NO: 129) genetically fused with rigid linkers A(EAAAK)₆ (SEQ ID NO: 161) to HSA-41 (SEQ ID NO: 116). ILF production in E. coli was performed as described in Example 3 using protein purified using affinity capture, IEX, and preparative SEC. Protein purity was assessed using SDS-PAGE and SEC-HPLC. The AFFIMER® polypeptides were found to be 99.8% to 100% pure (FIG. 14). Biacore kinetic analysis showed that genetically fused AFFIMER® dimers are able to engage both their target proteins (FIG. 15A). The AVA04 AFFIMER® polypeptide was found to bind PD-L1 and HSA-41 (SEQ ID NO:116) and engage HSA. Biacore analyses were carried out as described in Example 3 to analyze HSA binding and as described in Example 8 to analyze PD-L1-Fc binding (Table 10).

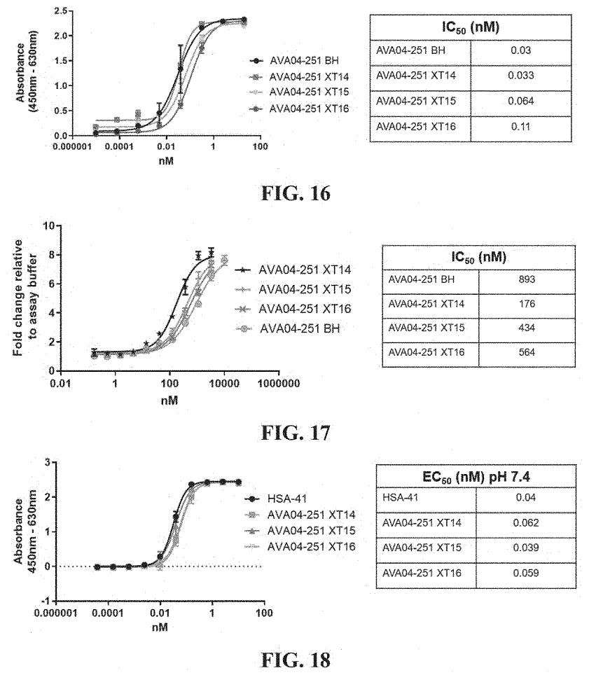

To evaluate if the addition of HSA-41 at various positions in an AFFIMER® in-line fusion format impacted binding of AVA04-251 to human PD-L1, a PD-L1 binding ELISA was performed with the three (3) ILF formatted AFFIMER® polypeptides (FIG. 16). Briefly, human PD-L1-Fc (R&D Systems) chimeric protein was coated on 96 well plates at 0.5 mg/ml in carbonate buffer. After saturation with 5% casein/PBS buffer, the plates were washed and a dilution of AFFIMER® polypeptides or controls were incubated for 90 minutes. Plates were then washed and a biotinylated polyclonal antibody anti-cystatin A (R&D Systems) was added for 1 hour. Plates were washed and AFFIMER® polypeptides were detected using streptavidin-HRP. After a last washing step, TMB was added for the development of the experiment and plates were read at 450 nm. The three (3) constructs tested exhibit similar EC₅₀ (ranging from 0.03 to 0.1 nM) and are identical to the anti-PD-L1 parental ILF dimer molecule (AVA04-251 BH (SEQ ID NO:129)). This was confirmed when a PD-1/PD-L1 blockade Bioassay (Promega) of the half-life extended AFFIMER® polypeptide compared to the parental molecule was performed (FIG. 17). The PD-1/PD-L1 blockade Bioassay (Promega) assay was run according to manufacturer instructions in duplicate and showed the three (3) constructs tested have similar EC₅₀ values (within 2-fold difference) and are identical to the parental dimer molecule (AVA04-251 BH; SEQ ID NO:129).

Similarly, the binding to human serum albumin was assessed for the three (3) half-life extended AFFIMER® polypeptides using an ELISA at pH 7.4. Briefly, HSA was coated in 96 well plates at 1 mg/ml at pH 7.5. After saturation with 5% PBS Casein pH 7.5, plates were washed and a dilution of AFFIMER® polypeptides or controls were incubated for 90 minutes. Plates were then washed, and a biotinylated polyclonal antibody anti-cystatin A (R&D Systems) was added for 1 hour. Plates were washed and AFFIMER® polypeptides were detected using streptavidin-HRP. After a last washing step, TMB was added for the development of the experiment and the plates were read at 450 nm. The three (3) constructs tested exhibit similar EC₅₀ (ranging from 0.03 to 0.06 nM) and are identical to the parental molecule (HSA-41; SEQ ID NO:116) (FIG. 18).

TABLE 10
BIACORE ™ Kinetic Analysis of AFFIMER ® Polypeptides (PD-L1-Fc, HSA)

| | rhPD-L1-Fc | | | | | HSA | | | | |
|---|---|---|---|---|---|---|---|---|---|---|
| AFFIMER ® | ka (1/Ms) | kd (1/s) | KD (nM) | Rmax (RU) | Chi² (RU²) | ka (1/Ms) | kd (1/s) | KD (nM) | Rmax (RU) | Chi² (RU²) |
| 251_BH | 7.52E+05 | 1.29E−03 | 1.71 | 23 | 0.154 | | | | | |
| parent | | | | | | | | | | |
| AVA04-251 | 8.34E+05 | 4.20E−04 | 0.504 | 24.9 | 0.355 | 3.22E+05 | 2.82E−03 | 8.75 | 73.3 | 0.211 |
| XT14 | | | | | | | | | | |
| AVA04-251 | 8.63E+05 | 6.07E−04 | 0.704 | 24.3 | 0.307 | 5.57E+05 | 2.90E−03 | 5.20 | 75.6 | 0.202 |
| XT15 | | | | | | | | | | |
| AVA04-251 | 4.03E+05 | 9.44E−04 | 2.34 | 34.2 | 0.194 | 8.21E+05 | 3.24E−03 | 3.95 | 78.5 | 0.228 |
| XT16 | | | | | | | | | | |
| HSA-41 | | | | | | 8.48E+05 | 4.35E−03 | 5.13 | 32.1 | 0.255 |
| parent | | | | | | | | | | |

### Example 10. Mixed Lymphocyte Reaction of Half-Life Extended ILF Trimer

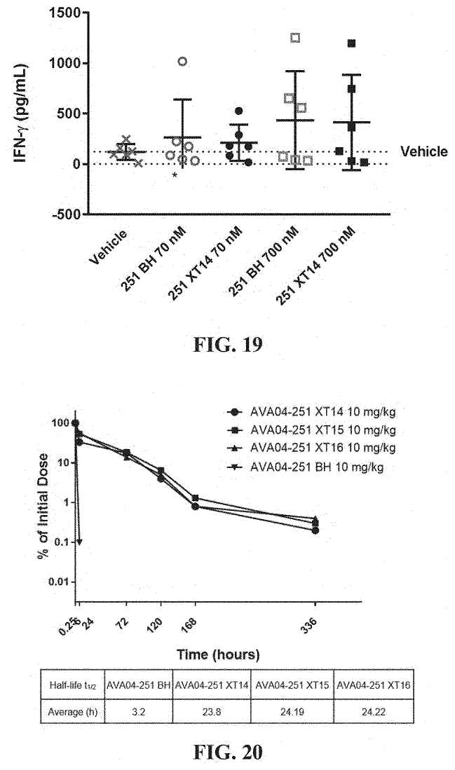

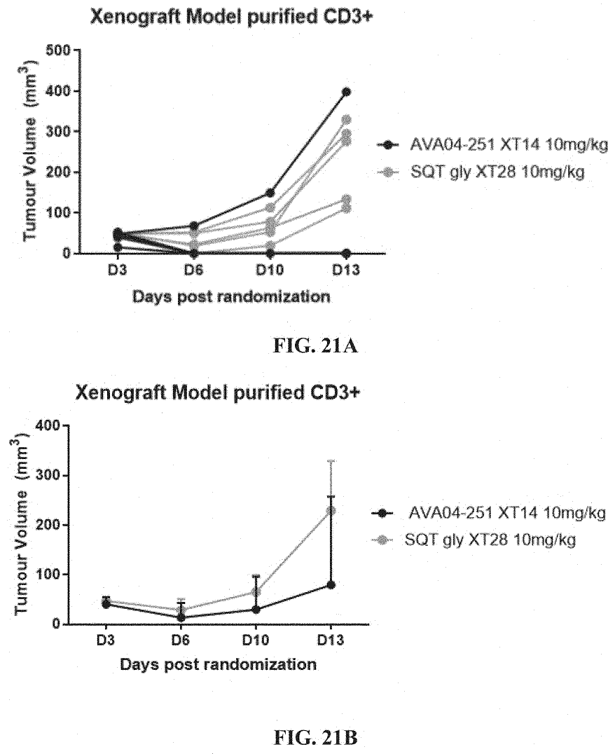

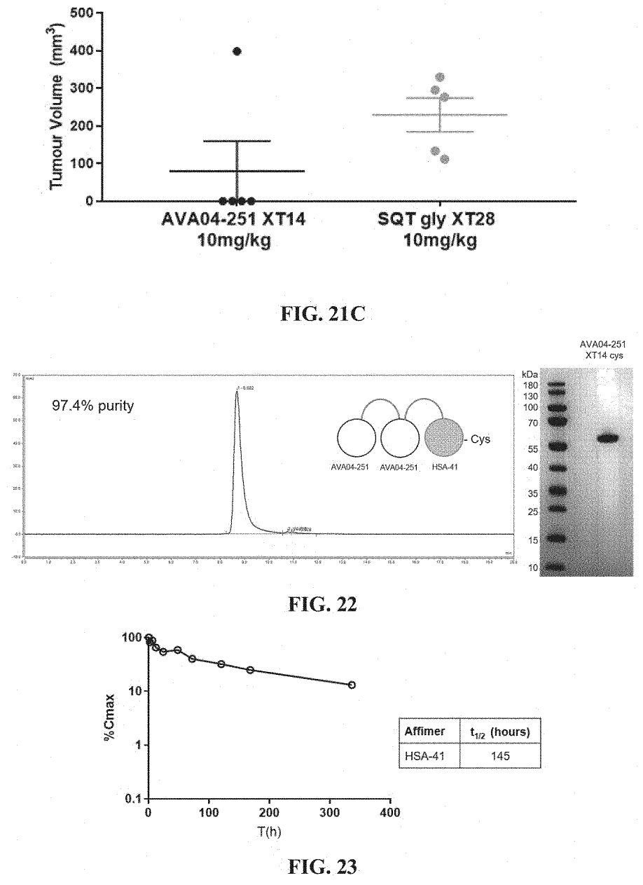

AVA04-251 XT in-line fusion formatted AFFIMER® polypeptides were tested in a mixed lymphocyte reaction (MLR) assay (FIG. 19). Briefly, dendritic cells (DC) derived from monocytes were prepared from CD14+ monocytes cultured for seven (7) days. Immature DCs were used on day 7 and cultured together with allogeneic T-cells (negative isolation) and reference substance or vehicle control (RPMI-10 media). Cells were cultured for 4 days and IFNγ was measured in supernatants at the end of the culture period by ELISA. Data are presented as mean +/−S.E.M. pg/ml or normalised to vehicle control (n=6). The dotted line represents mean vehicle (RPMI-10) value. The half-life formatted AFFIMER® polypeptide AVA04-251 XT14 (SEQ ID NO:123) was found to increase the level of IFNγ similarly to its control (non-half-life extended) (FIG. 21).

### Example 11. Pharmacokinetics Profile of PD-L1 Binding Half-Life Extended In-Line Fusion AFFIMER® Polypeptides in Mice

The ILF AVA04-251 trimers with half-life extension were tested in a pharmacokinetic study in C57/B16 mice. As described FIG. 20, mice were injected intravenously (IV) at 10 mg/kg. Six mice were used and serum was collected at nine time points (0, 0.25, 6, 24, 72, 120, 168, and 336 hours). The serum samples for each time point were pooled and analyzed by sandwich ELISA using the purified molecules injected as a reference standard. Results were expressed as the percentage of initial dose at 15 minutes. The AFFIMER® ILF protein without half-life extension (AVA04-251 BH SEQ ID NO:129) had a fast clearance (t_{1/2}3.2 hours) whereas ILF AVA04-251 XT formats all showed half-life extension, estimated in the beta phase (ranging from 23.8-24.2 hours).

### Example 12. Mouse Xenograft Model Testing of AFFIMER® ILF Trimer

PBMCs were isolated from one healthy donor. Total T-cells were isolated and expanded on A375 cells for two rounds for 7 to 10 days in complete medium supplemented with IL-2. Mice (n=10) were inoculated subcutaneously at the right flank region with A375 tumor cells and activated T-cells (0.2 ml in PBS) for tumor development. The treatments were started one-hour post cell inoculation. AVA04-251 XT14 (SEQ ID NO: 123) purified protein was administered two (2) times a week for three (3) weeks. Overall, tumor growth inhibition was shown for both treatments when compared to controls at day 13 post-randomization. More than 70% of mice treated with AVA04-251 XT14 (SEQ ID NO: 123) had a reduced tumor size compared to the control group, which was given the non-binding AFFIMER® polypeptide ILF SQT gly XT28 (SEQ ID NO: 128) (FIGS. 21A-21C).

### Example 13. AVA04 Half-Life Extended ILF Trimer C-Terminal Cys AFFIMER® Polypeptide Expression

The half-life extended trimer was synthesized to further comprise a C-terminal cysteine amino acid following the C-terminal 6xHis tag by quick change mutagenesis (Agilent) to create AVA04-251 XT14 cys (SEQ ID NO: 126). The AFFIMER® protein was produced from E. coli and purified with affinity, IEX, and preparative size exclusion as described in Example 3. Characterization of the purified protein under reducing conditions with 2 mM TCEP showed that the purity of the final protein is >97% (FIG. 22). AFFIMER® ILF proteins can therefore be produced with a free cysteine for subsequent conjugation using maleimide chemistry to enable the generation of AFFIMER® protein-drug conjugates.

### Example 14: Pharmacokinetic Profile of HSA-41 in Double Transgenic Mice Humanized for FcRn and Serum Albumin

As was previously demonstrated, the half-life of the HSA AFFIMER® protein is correlated to its binding affinity to serum albumin. HSA-41 (SEQ ID NO: 116) has significantly higher affinity for human serum albumin than mouse. Therefore, the PK profile of the lead molecule HSA-41 (SEQ ID NO: 116) was evaluated in a double transgenic humanized neonatal Fc receptor (FcRn)/human serum albumin mouse model to more closely mimic the physiological interactions found in humans. As described FIG. 23, mice were injected intravenously (IV) at 10 mg/kg. Nine mice were injected, and at 10 (ten) time points serum was collected (up to 336h). The serum samples for each time point were pooled and analyzed by sandwich ELISA using the molecules injected as reference standard. The results were expressed as percentage of concentration maximum. The half-life of the HSA-41 AFFIMER® polypeptide (SEQ ID NO: 116) protein was estimated in the beta phase at approximately 145 hours in this transgenic mouse model.

### Example 15: Pharmacokinetic Profile of 3 Lead AFFIMER® Polypeptides in Cynomolgus

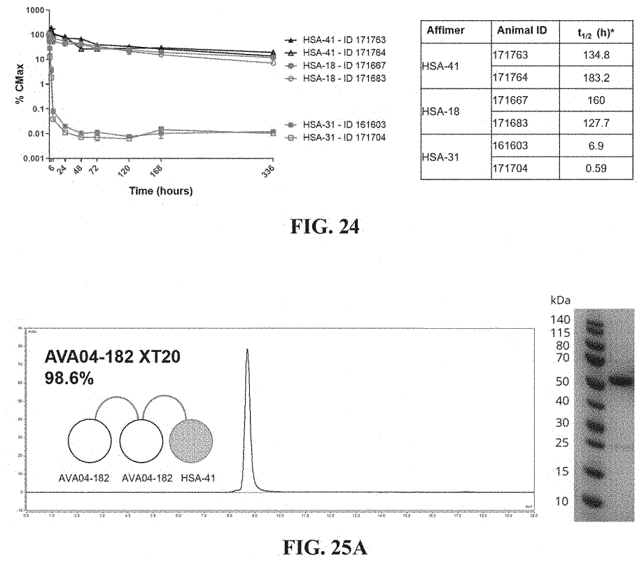

Three (3) AFFIMER® proteins, HSA-18 (SEQ ID NO: 110), HSA-31 (SEQ ID NO: 113), and HSA-41 (SEQ ID NO: 116), showed different PK profiles in mice. AFFIMER® polypeptides were administered at 5 mg/kg as a bolus intravenous (IV) injection in two (2) animals per group (one male and one female). Serum concentration of AFFIMER® protein was determined for 14 (fourteen) timepoints (0.25-672 hours) over 28 (twenty-eight) days by ELISA. All AFFIMER® proteins tested were well tolerated in vivo (FIG. 24 and Table 11).

TABLE 11
Pharmacokinetic Parameters in mouse of the AFFIMER ®
proteins (half-life, t_{1/2})

| Molecule | Animal ID | t_{1/2} (hours) |
|---|---|---|
| HSA-41 | 171763 | 134.8 |
| | 171764 | 183.2 |
| HSA-18 | 171667 | 160.0 |
| | 171683 | 127.7 |
| HSA-31 | 161603 | 6.9 |
| | 171704 | 0.59 |

### Example 16. Anti-Mouse PD-L1 binder half-life extended in-line fusion AFFIMER® trimer

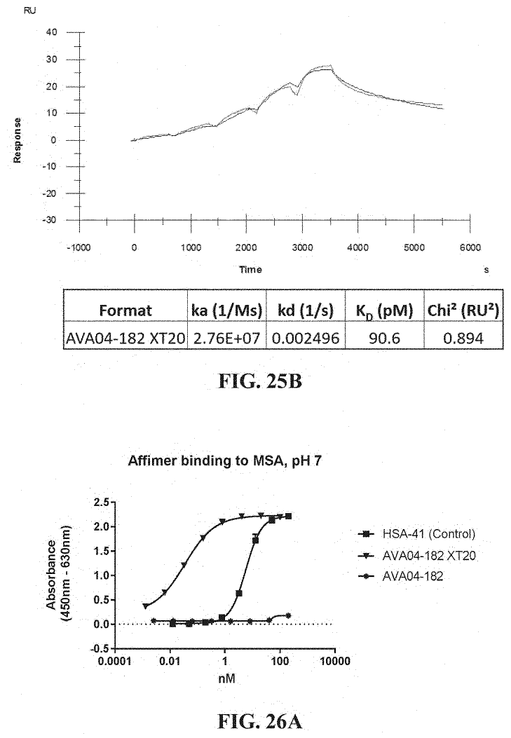

AVA04-182 XT20 (SEQ ID NO: 127) half-life extended ILF trimer was produced from E. coli. SDS-PAGE and SEC-HPLC analyses were run as described in Example 2 and showed final protein purity of over >98% (FIG. 25A). Purified protein was run on Biacore to assess its affinity to mouse PD-L1-Fc tagged recombinant antigen (R&D systems). Aantigen was captured using a Protein A chip (GE Healthcare) and the AFFIMER® ILF format was run as an analyte using single cycle kinetics titrating from a maximum concentration of 1 nM, and regenerating using 10 mM glycine pH 1.5 (GE Healthcare). Fc2-1 kinetic data was blank subtracted and fit to a 1:1 Langmuir binding model (BIAcore Evalution software; GE Healthcare) to calculate a K_{D} value of 90.6 pM, confirming that the addition of the half-life extending AFFIMER® polypeptide in this format did not affect the AVA04-182 binding to mouse PD-L1 target antigen (FIG. 25B).

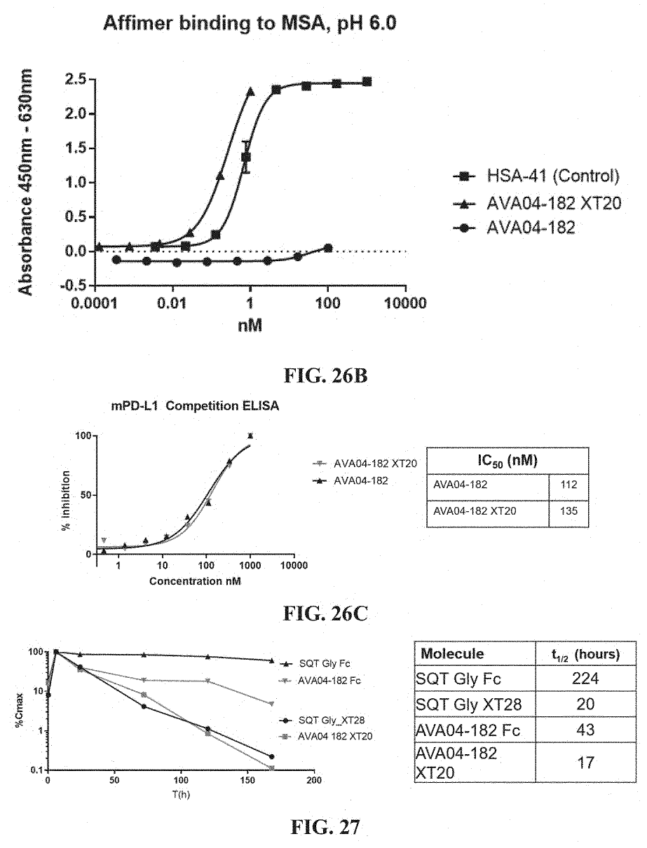

AVA04-182 XT20 (SEQ ID NO: 127) ILF was evaluated in an ELISA for its capacity to bind HSA at pH 7.4 and pH 6.0 (as described in the Example 4). FIGS. 26A and 26B shows that AVA04-182 XT20 retained the capacity of HSA-41 to bind MSA. In addition, to evaluate if the half-life extended AFFIMER® polypeptide was functional, a competitive ELISA (mPD-1/mPD-L1) was performed. Briefly, PD-1 was coated overnight on the plate at 1 μg/ml in carbonate buffer. Then plates were saturated using 5% Casein/PBS buffer. In the meantime, mPD-L1 was pre-incubated with a dilution of half-life extended AFFIMER® polypeptide and its control. After saturation, the mix was added to the plates and incubated for 90 minutes. Plates were then washed and the detection polyclonal antibody, biotinylated anti-PD-L1, was added. After washing the plates, streptavidin-HRP was added for 30 minutes. After a final wash, development of the reaction was performed using TMB (Pierce) and the plates were read using a plate reader at 450 nm (FIG. 26C). The figure shows that half-life extended AFFIMER® polypeptide has a similar neutralizing capacity to its parental molecule.

As a proof of concept, a pharmacokinetic study was performed in mice. Twelve animals per group were injected intraperiotneally (IP) with 25 mg/kg AFFIMER® polypeptide. Three animals were used per timepoint. At eight (8) timepoints, serum was drawn, up to 336h post injection. Pooled serum were analyzed using an ELISA to quantify the level of AFFIMER® polypeptide in serum. The pharmacokinetic profile of the half-life extended AFFIMER® polypeptide showed a half-life of ˜17 hours in this study (FIG. 27).

### Example 17: Biodistribution of AVA04-251 BH-800CW in a A375 Mouse Xenograft Model

Whether anti-PD-L1 AFFIMER® polypeptides are targeted to tumors expressing human PD-L1 was assessed in a mouse xenograft model examining the biodistribution of IR dye-conjugated AFFIMER® polypeptide over time using fluorescence imaging. AVA04-251 BH cys (SEQ ID NO:130) and AVA04-251 XT14 cys (SEQ ID NO: 126) were conjugated to IRDye 800CW (LI-COR) with maleimide chemistry to modify the accessible amino groups on the protein. AFFIMER® polypeptides were diluted to 1 mg/ml in 50 mM MES pH 6, 150 mM NaCl, 1 mM TCEP and incubated with IRDye 800CW (4 mg/mL in water) at a stoichiometry of 9:1 dye:protein for 2 hours in dark conditions at room temperature (˜23° C.). Free dye was separated from dye-conjugated AFFIMER® polypeptides using a 5 mL Zeba Spin Desalting Column (MWCO 7000; Pierce) according to the manufacturer's instructions. The dye:protein ratio was calculated based on the absorbance at 280 and 780 nm according to the equation:

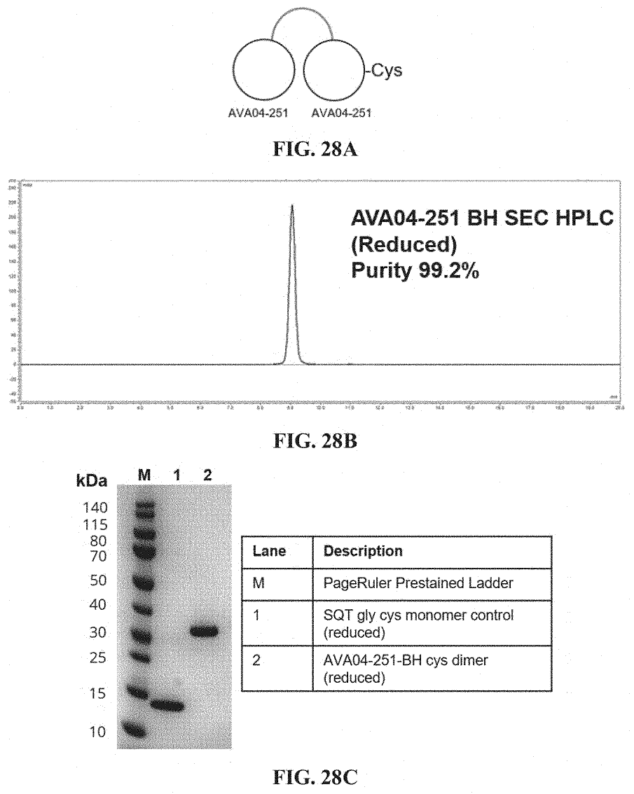

Dye:protein ratio=(A780/εDye)/(A280−(0.03×A780))/εprotein,

where 0.03 is the correction factor for the absorbance of IRDye 800CW at 280 nm, and εDye and ε protein are molar extinction coefficients for the dye 270,000 M⁻¹ cm⁻¹ and protein 39871 M⁻¹ cm⁻¹ for AVA04-251 BH Cys (SEQ ID NO: 130) and 37626 M⁻¹ cm⁻¹ for AVA04-251 XT14 cys (SEQ ID NO: 126), respectively. FIGS. 28A-28C show the format schematic and purity of conjugated material using SEC-HPLC and SDS-PAGE analytical methods (as detailed in Example 2).

The binding of dye-conjugated AVA04-251 BH-800 or AVA04-251 XT14-800 to recombinant human PD-L1 was compared to non-conjugated AFFIMER® polypeptide using a PD-L1 binding ELISA.

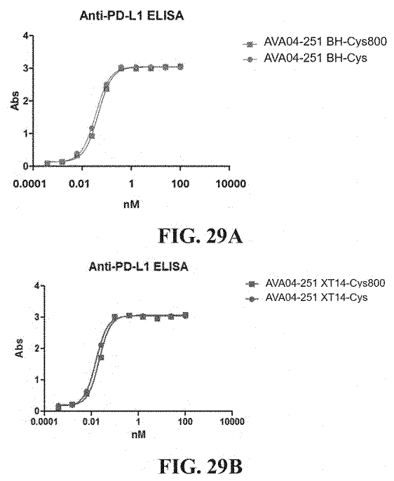

Briefly, human PD-L1 Fc (R&D Systems) chimeric protein was coated onto 96 well plates at 0.5 μg/mL in carbonate buffer. After saturation with 5% casein/PBS buffer, plates were washed and a dilution of conjugated AFFIMER® polypeptide or unconjugated control were incubated for 90 minutes. Plates were then washed, a biotinylated polyclonal anti-cystatin A antibody (R&D Systems) added, and the plates incubated for 1 hour. Plates were washed and bound AFFIMER® polypeptide was detected using streptavidin-HRP. After a last washing step, TMB was added and the plate was read at 450 nm. The conjugated AFFIMER® polypeptide exhibited a similar EC₅₀ compared to the parental molecule. Therefore, the data indicate that dye conjugation does not impact the affinity of both conjugated formatted molecules for the PD-L1 target based on comparable binding curves (FIG. 29).

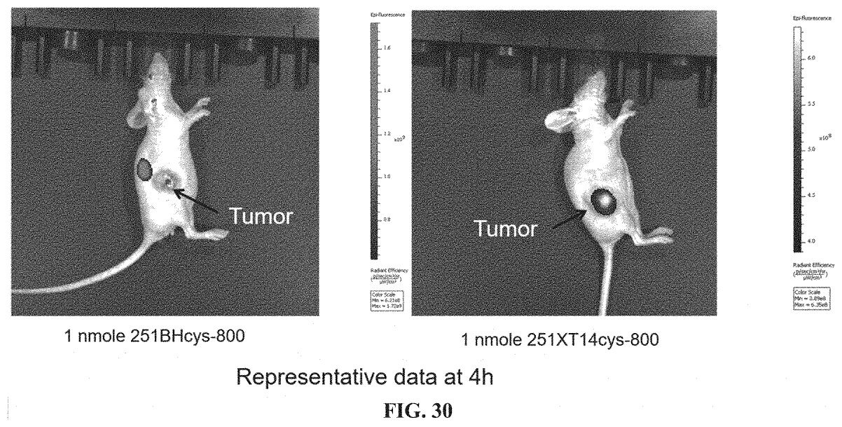

The A375 mouse xenograft model was established in female athymic nude mice (Charles River Laboratories) following subcutaneous injection of A375 cells (5×10⁶ cells [ATCC] in 100 μL sterile PBS) into the animal's flank. Tumors were monitored three (3) times per week, with the developing tumor being measured with calipers. Tumors were allowed to grow between 500-1000 mm³ prior to intravenous administration of AVA04-251 BQ-800 and BH-800 (at 1 nmole) into the tail vein of three (3) mice. Fluorescence images were recorded with a Xenogen IVIS 200 Biophotonic Imager immediately after injection (time 0) and at 1, 2, 4, 8, 24, and 48 hours post-dose. At the four (4) hour timepoint, targeting of the anti-PD-L1 AFFIMER® polypeptide with half-life extension to the tumor was detected. The data are presented in FIG. 30, and arrows indicate the approximate locations of the tumor.

### Example 18: Crystallization of HSA-41 l AFFIMER® Polypeptide in Complex with HSA

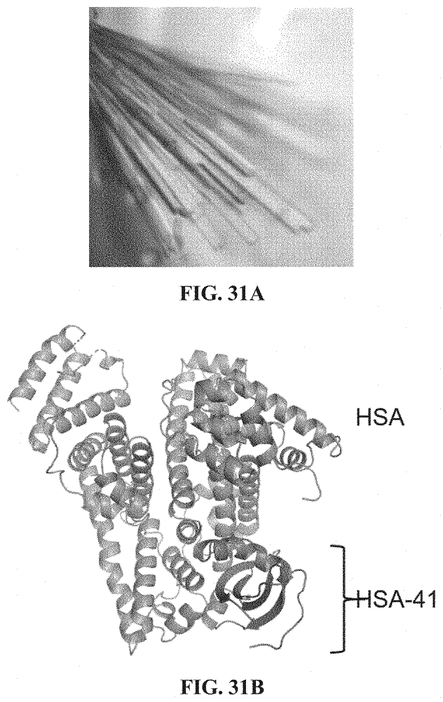

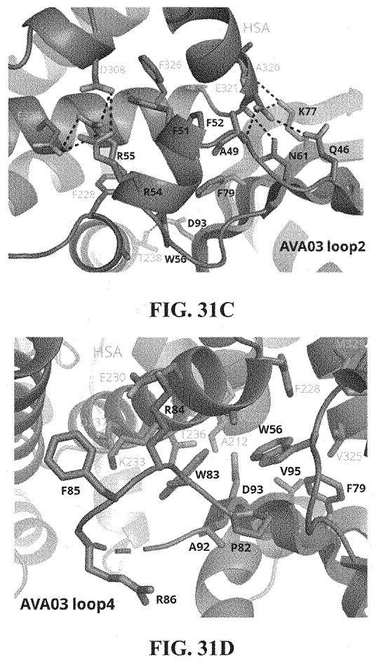

HSA-41 (SEQ ID NO: 116) was expressed and purified using NiNTA and preparative size exclusion chromatography from BL21 E. coli cells as described in Example 2. HSA was purchased from Sigma Cat. No. A3782 and reconstituted to 50 mg/ml. Purified AFFIMER® polypeptide was mixed at 1:1.5 molar ratios of HSA for 1 hour with gentle agitation. The protein complex formed was purified using preparative size exclusion chromatography using 10 mM Tris pH 7.4 and 150 mM NaCl buffer as a mobile phase. Eluted complex fractions of the correct molecular weight were concentrated to 105.3 mg/ml, snap frozen on liquid nitrogen and stored at −80C. To perform the crystallography, several commercial screens were set up at two temperatures: +4° C. and +18° C. The sitting drop vapor diffusion method using 100:100 nl protein to reservoir solution was performed. The crystallization screens set up were: MD JCSG+, MD PACT, MD Proplex, MD Structure, and Hampton Salt RX. Diffraction datasets were collected on crystals (FIG. 31A) obtained from 10 mM nickel (II) chloride hexahydrate, 0.1 M Tris-HCl pH 8.5 and 20% w/v PEG 2000 MME-produced crystals. Data was collected with a Diamond light source, UK. The diffraction dataset is near complete to 3.05 A2 (3D crystal structure of protein complex FIG. 31B). HSA-41 (SEQ ID NO: 116) was shown to bind domain II of HSA mainly through AFFIMER® polypeptide loop 2 interactions, as the electron density indicates covalent modification by Ni²⁺ ions on the surface of the proteins and likely, facilitated crystallization. The overall interaction area of 880 Å² is mainly through binding loop 2 which was found to form an alpha helix structure within the loop, and the interface is characterized by a mixture of hydrophilic and hydrophobic interactions. Loop 4 picks up few interactions. FIGS. 31C, 31D and Table 12 show the specific amino acid interactions between the AFFIMER® polypeptide and HSA.

TABLE 12
Crystal structure predicted amino acid interactions of HSA-41
AFFIMER ® polypeptide with HSA

| | HSA antigen | HSA-41 |
|---|---|---|
| Type of interaction | amino acid | AFFIMER ® amino acid |
| Hydrogen bond | T236 | D93 |
| Hydrogen bond | A320 | K77 |
| Hydrogen bond | E321 | Q46/A49/N61/K77 |
| Salt bridge | D308/E333 | R54/R55 |
| Hydrophobic interaction | L48 | F228 |
| Hydrophobic interaction | F51 | A229 |
| Hydrophobic interaction | F52 | A322 |
| Hydrophobic interaction | W56 | V325 |
| Hydrophobic interaction | F79 | F326 |
| Hydrophobic interaction | V95 | M329 |

### Example 19: HSA-41 Alanine Scanning Mutant Summary of Loop 2 & 4

The AFFIMER® polypeptide HSA-41 (SEQ ID NO. 116) was mutated using site-directed mutagenesis to alanine residues throughout each amino acid in loop 2 and loop 4 in order to identify which amino acids were engaging target antigen. Final clones were sequence-verified, produced from E. coli, and affinity purified as described in Example 3. A total of eighteen (18) alanine mutants were compared following one stage purification on SEC-HPLC for protein purity and binding response to HSA at pH7.4 on Biacore at 50 nM (standard AFFIMER® protein concentration). Data showed loop 2 is heavily involved in binding to target with residues 51,52, 55, 56, and 58 losing binding signal when mutated to alanine. In loop 4, the first position 84 loses its ability to bind when mutated to alanine, and the rest of the loop is less involved in binding (Table 13). SEC-HPLC data showed loop 2 positions 50 and 55 may be involved in self association, as protein purity decreased when either was substituted for alanine.

TABLE 13
Results of Alanine Mutant Screening

| | | | | |
|---|---|---|---|---|
| NA | HSA-41 AH | 116 | 36.8 | N/A |
| | parent | | | |
| 2 | N50A | 143 | 13 | 36.512 |
| 2 | F51A | 144 | 2.8 | 80.72 |
| 2 | F52A | 145 | 2.2 | 72.01 |
| 2 | Q53A | 146 | 23.5 | 84.08 |
| 2 | R54A | 147 | 11.9 | 72.18 |
| 2 | R55A | 148 | 2.9 | 50.51 |
| 2 | W56A | 149 | 2.6 | 90.10 |
| 2 | P57A | 150 | 23.2 | 77.36 |
| 2 | G58A | 151 | 6.5 | 73.91 |
| 4 | W83A | 152 | 2.9 | 89.35 |
| 4 | K84A | 153 | 23.5 | 84.51 |
| 4 | F85A | 154 | 26.6 | 90.41 |
| 4 | R86A | 155 | 19.1 | 88.02 |
| 4 | N87A | 156 | 25.8 | 85.22 |
| 4 | T88A | 157 | 27.9 | 85.43 |
| 4 | D89A | 158 | 26.1 | 94.13 |
| 4 | R90A | 159 | 17.2 | 78.86 |
| 4 | G91A | 160 | 17.8 | 79.01 |

### Example 20: HSA-41 Loop 4 Knockout Mutants

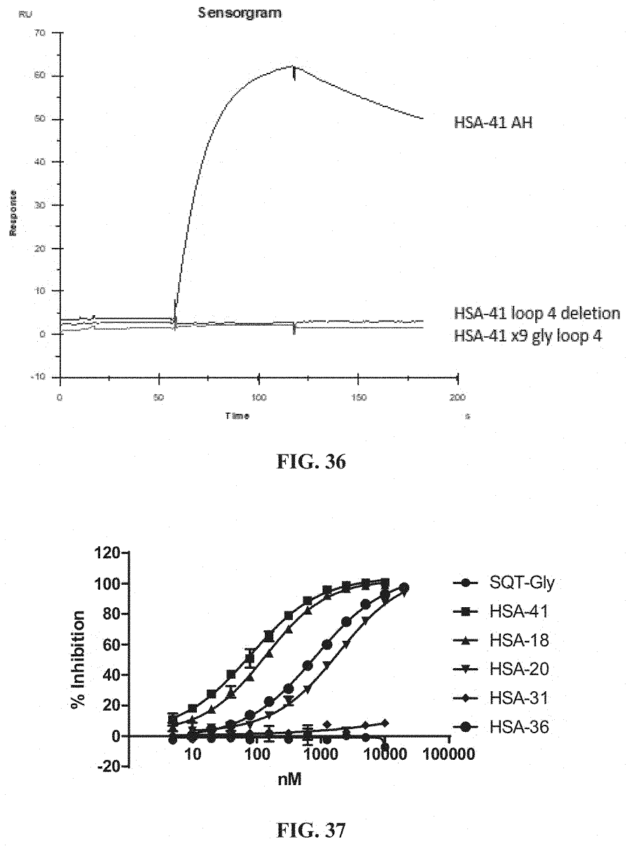

From the solved crystal structure of HSA −41 (SEQ ID NO: 116) (Example 18) and alanine scanning (Example 19) experiments, HSA-41 was shown to bind HSA predominantly through loop 2. AFFIMER® polypeptides were designed to knockout loop 4 with either a deletion (SEQ ID NO: 141) or by replacing the loop with 9 glycine residues (SEQ ID NO: 142). These mutants lost the ability to bind to the target antigen, demonstrating that loop 4 is needed for HSA-41 to engage target and for half-life extension (FIG. 36).

### Example 21: Avidity of ILF Homodimers of HSA-41

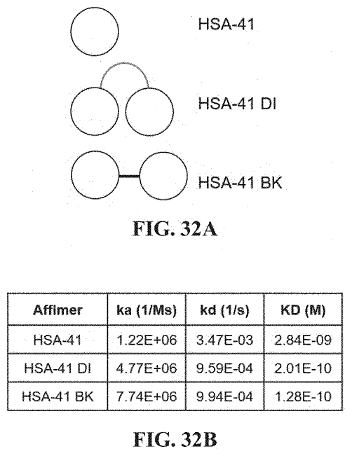

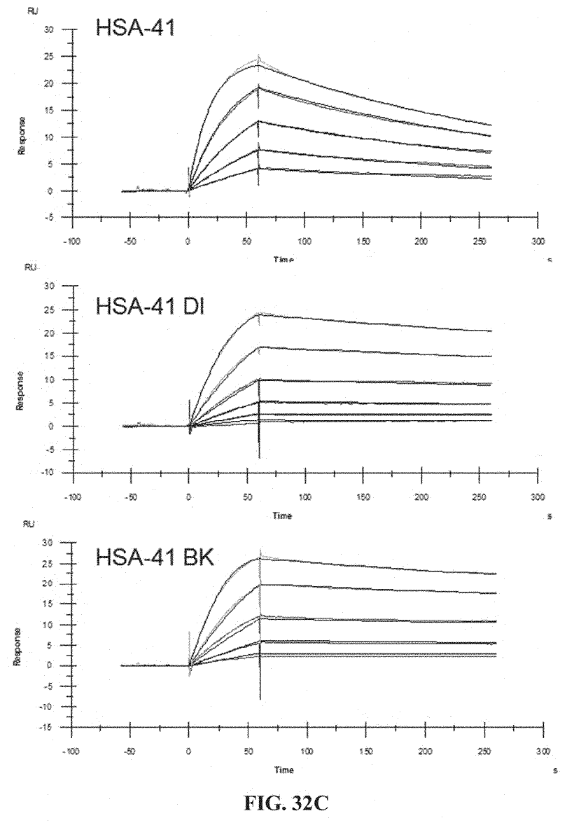

AFFIMER® polypeptides were genetically fused to form ILF homodimers with rigid (HSA-41 BK; SEQ ID NO: 131) or flexible (HSA-41 DI; SEQ ID NO: 132) repetitive linkers (schematic illustrations FIG. 32A). The AFFIMER® polypeptides were produced and purified from E. coli as described in Example 3. Biacore kinetic analysis was performed at pH 7.4 for binding to immobilized HSA (as described in Example 3). The analysis showed avidity when AFFIMER® polypeptides were fused, with pM K_{D} values compared to nM monomer binding to HSA (FIGS. 32B and 32C).

### Example 22: HSA-41 Incubations with Serum Albumin, SEC-HPLC Characterization

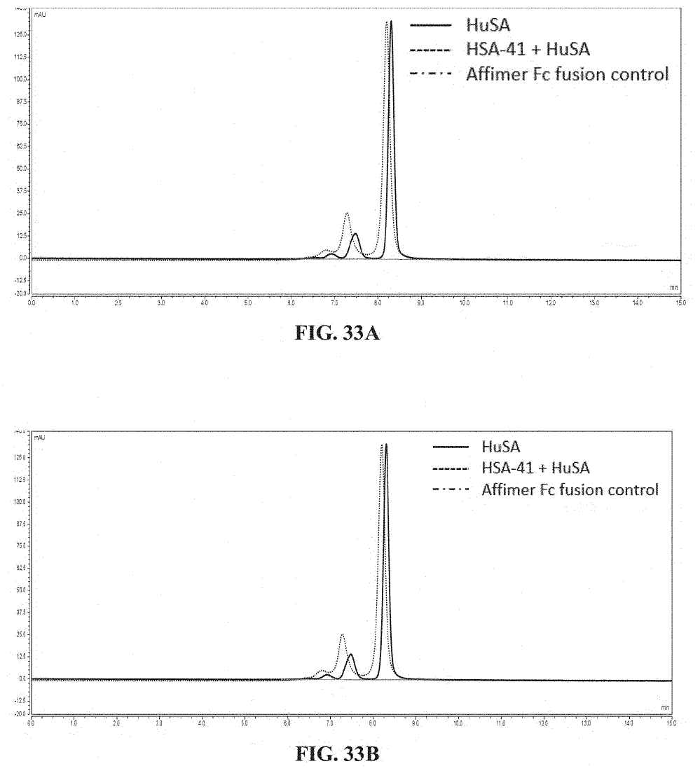

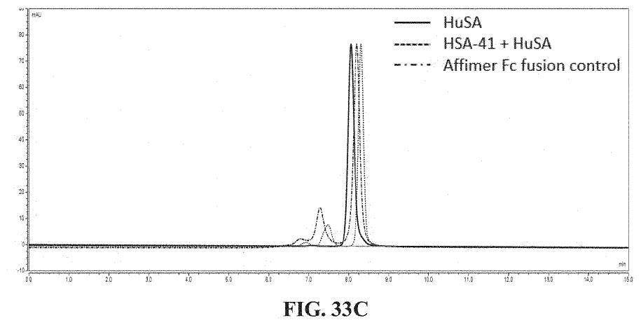

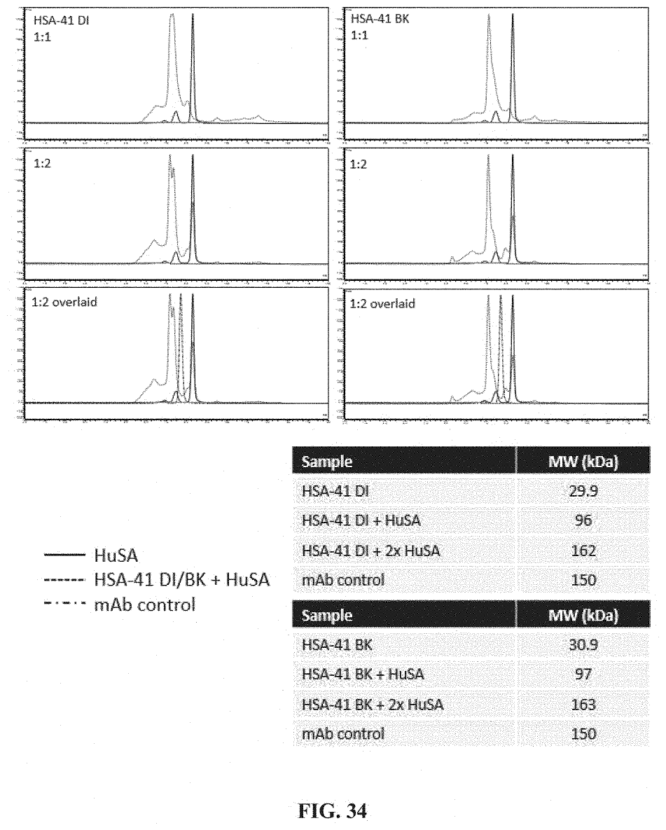

The AFFIMER® polypeptide, HSA-41 (SEQ ID NO: 116), was incubated with HSA (Sigma) at a 1:1 or a 1:2 ratio over a time course of one to four hours. The total mass of the HSA-41 AFFIMER®:HSA complex was compared to mass controls of an AFFIMER®-Fc fusion protein (80.5 kDa) run on a SEC-HPLC Acclaim −300 column (Thermo). Results show an expected molecular weight (MW) of 83 kDa of the complex with a 1:1 binding stoichiometry after one hour (FIGS. 33A-33C). The same experiments were performed with incubations of the dimer in-line fusion (ILF) protein formats: HSA-41 DI (SEQ ID NO: 132) or HSA-41 BK (SEQ ID NO: 131). The ILF proteins were incubated with HSA over four hours (FIG. 34). SEC-HPLC analysis of these samples was performed on a Yarra-3000 (Phenomenex) column, and data showed a 2:1 binding stoichiometry of HSA:AFFIMER® ILF dimer with both AFFIMER® polypeptides in the ILF format engaging HSA simultaneously. The ILF dimer:HSA complexes ran with a mass of ˜160 kDa running at a higher MW than a monoclonal antibody mass control of 150 kDa on the column (FIG. 34).

### Example 23: Pharmacokinetic Analysis of HSA-41 Monomer and ILF Dimer in C57BL/6 Mice

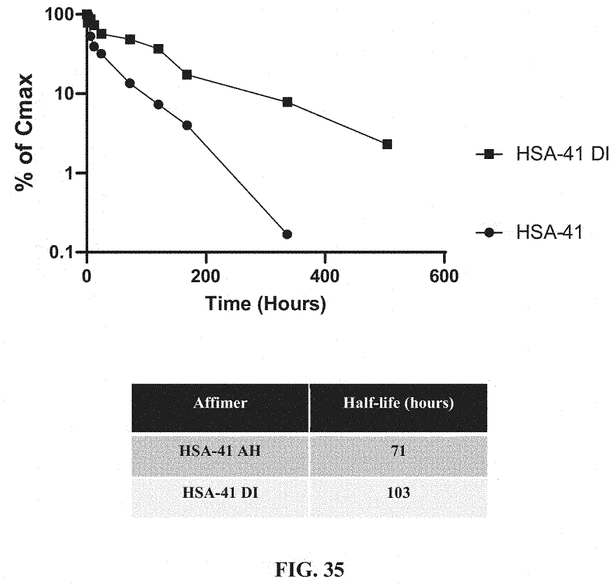

Nine (9) wild type C57BL/6 mice were injected intravenously with 10 mg/kg AFFIMER® polypeptide, and blood samples were collected at 10 timepoints (0.25 min and 2, 6, 12, 24, 72, 120, 168, 336 and 504 hours). Sera were prepared and frozen until analysis. For each timepoint, sera were pooled and AFFIMER® polypeptides were detected and quantified using an anti-cystatin sandwich ELISA. Analyzed data showed half-life extension calculated from the beta-phase of the AFFIMER® monomer (72h) was further increased with the ILF dimer HSA-41 DI (103h) in wild type mice (FIG. 35).

### Example 24: Epitope Binning of Anti-Serum Albumin Binders

A Homogeneous Time Resolved Fluorescence (HTRF) assay was used for epitope binning. The assay was used to screen serum albumin-binding AFFIMER® polypeptides for binding to biotinylated human serum albumin bound to HSA-41 Myc His. The interaction between HSA-41 Myc His and biotinylated HSA was performed using streptavidin labelled with terbium cryptate donor and anti-Myc labelled with d2 acceptor. All AFFIMER® proteins tested in the competition assay were produced without Myc tags (HSA-18, -20, and -36) and showed inhibition of the interaction between HSA-41 Myc His and biotinylated HSA, except for HSA-31), suggesting it binds to a different epitope (FIG. 37).

### Example 25: HSA-41 Free C-Terminal Cysteine Format (CQ) Characterization

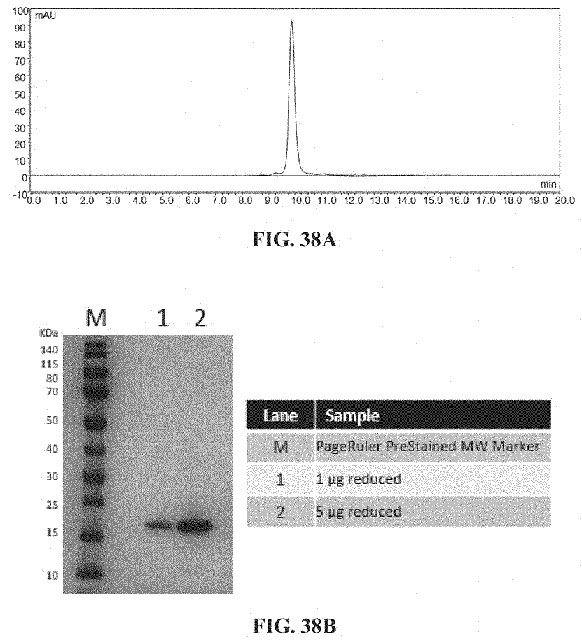

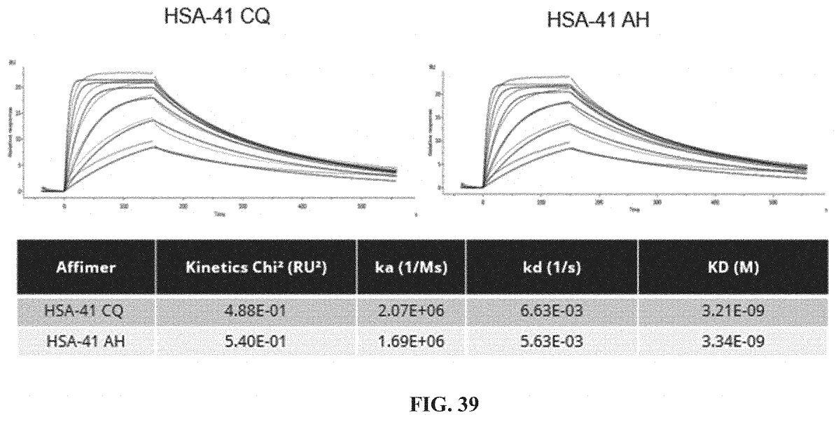

The AFFIMER® polypeptide HSA-41 (SEQ ID NO: 116) was genetically engineered to insert a free cysteine residue at the C-terminal tag region of HSA-41, generating HSA-41 CQ (SEQ ID NO. 138), which is expressed with the C-terminal tags Myc Cys TEV His. HSA-41 CQ can be used for conjugation via maleimide chemistry, for example, to add polypeptide's half-life. The Cys variant AFFIMER® polypeptide (HAS-41 CQ) was purified in the presence of the reducing agent 5 mM TCEP and characterized on SDS-PAGE and SEC-HPLC under reducing conditions (FIGS. 38A-38B). Biacore kinetic analysis for binding to HSA at pH7.4 was performed as described in Example 3. HSA-41 CQ was found to be comparable to the monomer without a free C-terminal Cys, having a KD of ˜3 nM (FIG. 39).

### Example 26: AVA04-251 XT ILF Formatting with HSA-18 Half-Life Extending AFFIMER® Polypeptides

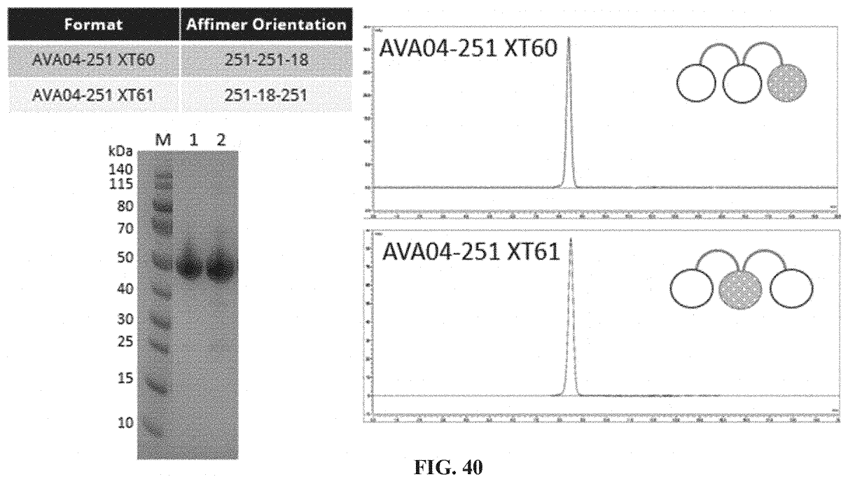

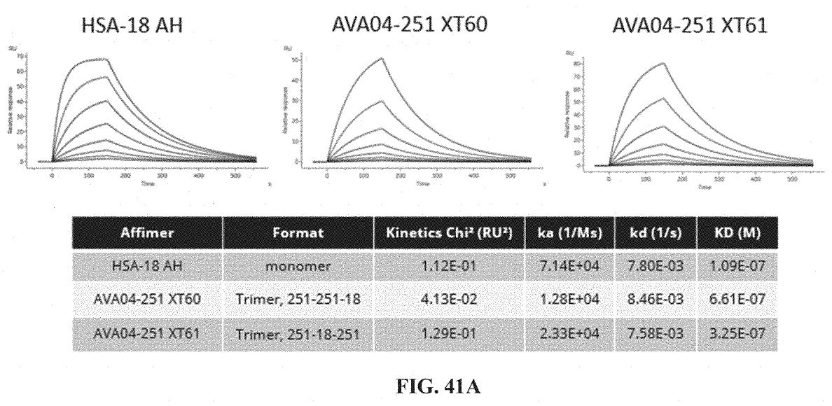

Two AFFIMER® trimeric in-line fusion (ILF) formats were designed. Each comprised two fused AVA04-251 human PD-L1 binding AFFIMER® polypeptides, which were further fused with HSA-18 (SEQ ID NO: 110) to extend half-life. AVA04-251 XT60 (SEQ ID NO. 139) comprised the half-life extending AFFIMER® polypeptide positioned at the C-terminus, whereas AVA04-251 XT61 (SEQ ID NO. 140) comprised the half-life extension AFFIMER® polypeptide in the middle of the format, separating the two anti-PD-L1 AFFIMER® polypeptides (schematic diagrams, FIG. 40). Formats were designed with repetitive rigid genetic linkers A(EAAAK)₆ (SEQ ID NO: 161) between AFFIMER® polypeptides. AFFIMER® trimers were produced from E. coli and purified with affinity NiNTA resin followed by preparative size exclusion as described in Example 2. Reducing SDS-PAGE and SEC-HPLC analysis show the final purity of the protein formats was >98% (FIG. 40).

### Example 27: Half-Life Extended AVA04-251 XT60 and AVA04-251 XT61 ILF Format Binding to Serum Albumin

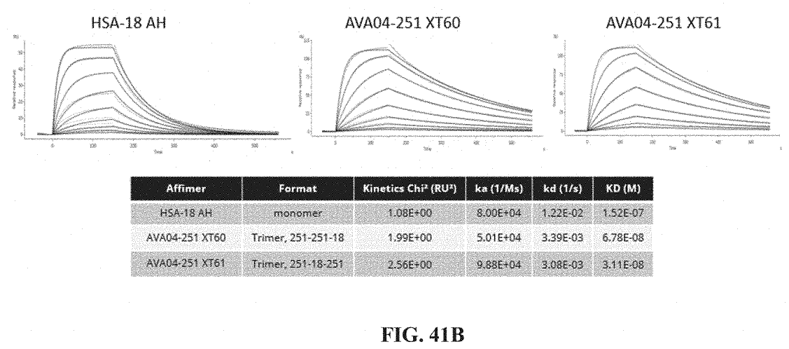

Human serum albumin (HSA) Biacore kinetic analysis was performed with pH6.0 and with pH7.4 running buffer using the method previously described in Example 3. Data showed the ILF formats containing the half-life extending AFFIMER® polypeptide HSA-18 (SEQ ID NO: 110) bound HSA with a KD of triple digit nM affinity at pH7.4 and double digit nM affinity at pH6.0, within 2-4 fold of the HSA-18 monomer affinity of 109-152 nM (FIGS. 41A-41B). For mouse serum albumin (MSA) at pH6.0 conditions, binding affinity of the ILF formats was within approximately 2-fold of the monomer serum albumin binding AFFIMER® polypeptide (FIG. 43).

### Example 28: Half-Life Extended AVA04-251 XT60 and AVA04-251 XT61 ILF Format Binding to Serum Albumin

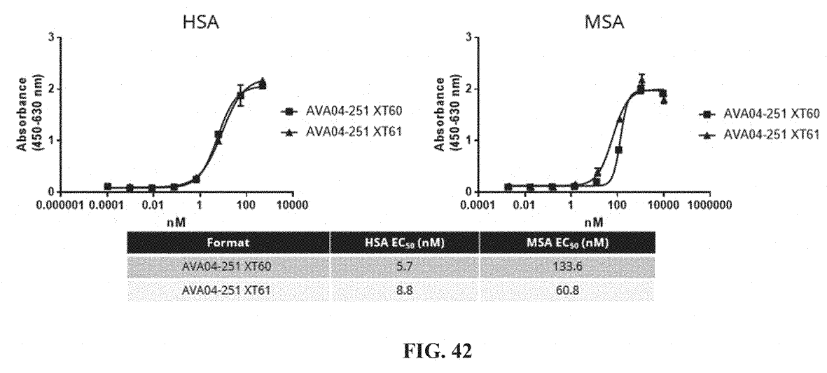

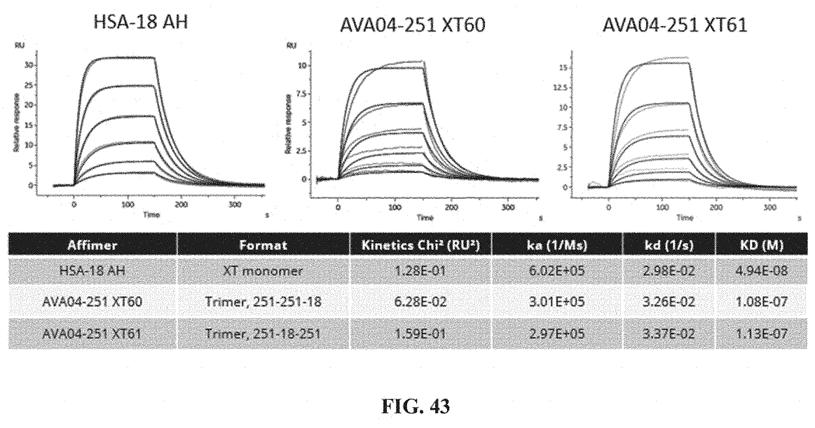

Binding to human serum albumin and mouse serum albumin was assessed for the two half-life extended ILF AFFIMER® formats (AVA04-251 XT60, SEQ ID NO: 139; AVA04-251 XT61, SEQ ID NO: 140) at pH 7.4 with an ELISA. Briefly, HSA or MSA was coated in 96 well plates at 1 mg/ml at pH 7.5. After saturation with 5% PBS Casein pH 7.5, plates were washed and a dilution of AFFIMER® trimers or controls were incubated on the plate for 90 minutes. Plates were then washed, and a biotinylated polyclonal antibody anti-cystatin A (R&D Systems) was added for 1 hour. Plates were washed and AFFIMER® ILFs were detected using streptavidin-HRP. After a last washing step, TMB was added for the development of the experiment, and the plates were read at 450 nm. The two ILFs tested, AVA04-251 XT60 and AVA04-251 XT61, exhibited similar EC₅₀ values for both HAS (ranging from 5.7 to 8.8) and MSA (ranging from 133.6 to 60.8) (FIG. 42).

### Example 29: AVA04-251 XT60 and AVA04-251 XT61 ILF Format Binding to Human PD-L1-Fc

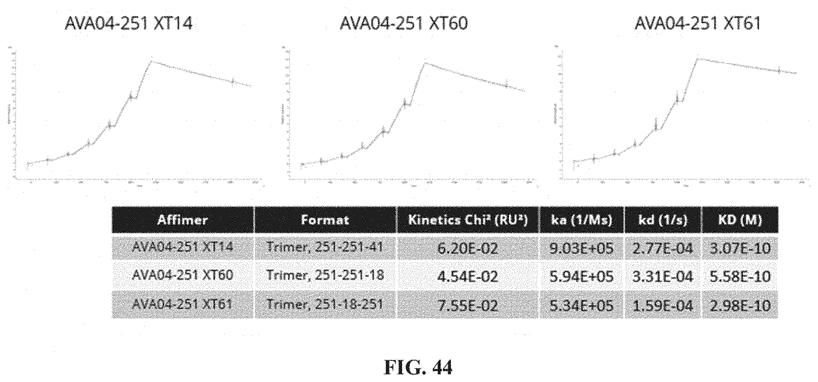

Biacore kinetic analysis was performed with single cycle kinetics to assess binding of AVA04-251 XT60 and AVA04-251 XT61 (SEQ ID NOs: 139 and 140, respectively) as described in Example 3. The experiments were performed to compare the AFFIMER® trimers to HSA-41. Binding affinity KD values were in the triple digit nM range, with similar on and off rates observed, regardless of whether the half-life extending AFFIMER® polypeptide was in the middle or C-terminal end of the format (FIG. 44).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

1. A polypeptide that binds to human serum albumin (HSA) with a K_{d} of 1×10⁻⁶ M or less at pH 7.4, and at pH 6 binds to HSA with a K_{d} that is at least half a log less than the K_{d} for binding to HSA at pH 7.4.

2. The polypeptide of claim 1, wherein the polypeptide binds to HSA with a K_{d} of 1×10⁻⁷ M or less at pH 7.4, a K_{d} of 1×10⁻⁸ M or less at pH 7.4, or K_{d} of 1×10⁻⁹ M or less at pH 7.4.

3. The polypeptide of claim 1 or 2, wherein the polypeptide at pH 6 binds to HSA with a K_{d} that is at least one log less than the K_{d} for binding to HSA at pH 7.4, at least 1.5 logs less than the K_{d} for binding to HSA at pH 7.4, at least 2 logs less than the K_{d} for binding to HSA at pH 7.4, or at least 2.5 log less than the K_{d} for binding to HSA at pH 7.4

4. The polypeptide of any one of claim 1-3, wherein the polypeptide has a serum half-life in human patients of greater than 10 hours, greater than 24 hours, greater than 48 hours, greater than 72 hours, greater than 96 hours, greater than 120 hours, greater than 144 hours, greater than 168 hours, greater than 192 hours, greater than 216 hours, greater than 240 hours, greater than 264 hours, greater than 288 hours, greater than 312 hours, greater than 336 hours or, greater than 360 hours.

5. The polypeptide of any one of claims 1-4, wherein the polypeptide has a serum half-life in human patients of greater than 50%, greater than 60%, greater than 70%, or greater than 80% of the serum half-life of HSA.

6. The polypeptide of any one of claims 1-5 comprising an amino acid sequence represented in general formula (I)
FR1-(Xaa)ₙ-FR2-(Xaa)ₘ-FR3 (I),
wherein
FR1 is an amino acid sequence having at least 70% identity to MIPGGLSEAK PATPEIQEIV DKVKPQLEEK TNETYGKLEA VQYKTQVLA (SEQ ID NO: 1);
FR2 is an amino acid sequence having at least 70% identity to GTNYYIKVRA GDNKYMHLKV FKSL (SEQ ID NO: 2);
FR3 is an amino acid sequence having at least 70% identity to EDLVLTGYQV DKNKDDELTG F (SEQ ID NO: 3); and
Xaa, individually for each occurrence, is an amino acid,
n is an integer from 3 to 20, and m is an integer from 3 to 20.

7. The polypeptide of claim 6, wherein:
FR1 has at least 80%, at least 82%, at least 84%, at least 86%, at least 88%, at least 90%, at least 92%, at least 94%, at least 96%, or at least 98% identity to SEQ ID NO: 1;
FR2 has at least 80%, at least 84%, at least 88%, at least 92%, or at least 96% identity to SEQ ID NO: 2; and/or.
FR3 has at least 80%, at least 85%, at least 90%, or at least 95% identity to SEQ ID NO: 3.

8. The polypeptide of claim 7, wherein:
FR1 comprises the amino acid sequence of SEQ ID NO: 1;
FR2 comprises the amino acid sequence of SEQ ID NO: 2; and/or
FR3 comprises the amino acid sequence of SEQ ID NO: 3.

9. The polypeptide of claim 6, wherein the amino acid sequence is represented in general formula (II)
(SEQ ID NO: 166)
MIP-Xaa1-GLSEAKPATPEIQEIVDKVKPQLEEKTNETYGKLEAVQYKT
QVLA-(Xaa)ₙ-Xaa2-TNYYIKVRAGDNKYMHLKVF-Xaa3-Xaa4-
Xaa5-(Xaa)ₘ-Xaa6-D-Xaa7-VLTGYQVDKNKDDELTGF,
(II)
wherein
Xaa, individually for each occurrence, is an amino acid;
n is an integer from 3 to 20, and m is an integer from 3 to 20;
Xaa1 is Gly, Ala, Val, Arg, Lys, Asp, or Glu;
Xaa2 is Gly, Ala, Val, Ser or Thr;
Xaa3 is Arg, Lys, Asn, Gln, Ser, Thr;
Xaa4 is Gly, Ala, Val, Ser or Thr;
Xaa5 is Ala, Val, Ile, Leu, Gly or Pro;
Xaa6 is Gly, Ala, Val, Asp or Glu; and
Xaa7 is Ala, Val, Ile, Leu, Arg or Lys.

10. The polypeptide of claim 9, wherein the amino acid sequence is represented in general formula (III)
(SEQ ID NO: 167)
MIPRGLSEAKPATPEIQEIVDKVKPQLEEKTNETYGKLEAVQYKT
QVLA-(Xaa)n-STNYYIKVRAGDNKYMHLKVFNGP-(Xaa)m-ADR
VLTGYQVDKNKDDELTGF,
(III)
wherein
Xaa, individually for each occurrence, is an amino acid; n is an integer from 3 to 20; and m is an integer from 3 to 20.

11. The polypeptide of any one of claims 6-10, wherein (Xaa)ₙ is represented by formula (IV)
(SEQ ID NO: 180)
aa1-aa2-aa3-aa4-aa5-aa6-aa7-aa8-aa9,
(IV)
wherein
aa1 is an amino acid selected from D, G, N, and V;
aa2 is an amino acid selected from W, Y, H, and F;
aa3 is an amino acid selected from W, Y, G, W, and F;
aa4 is an amino acid selected from Q, A, and P;
aa5 is an amino acid selected from A, Q, E, R, and S;
aa6 is an amino acid selected from K, R, and Y;
aa7 is an amino acid selected from W and Q;
aa8 is an amino acid selected from P and H; and
aa9 is an amino acid selected from H, G, and Q.

12. The polypeptide of any one of claims 6-11, wherein (Xaa)ₙ is an amino acid sequence having at least 80% or at least 90% identity to the amino acid sequence of any one of SEQ ID NOS: 4-55.

13. The polypeptide of claim 12, wherein (Xaa)ₙ is the amino acid sequence of any one of SEQ ID NOS: 4-55.

14. The polypeptide of any one of claims 6-13, wherein (Xaa)ₙ is an amino acid sequence having at least 80% or at least 90% identity to the amino acid sequence of any one of SEQ ID NOS: 22, 24, 26, 35, 40, 41, and 45.

15. The polypeptide of claim 14, wherein (Xaa)ₙ is an amino acid sequence of any one of SEQ ID NOS: 22, 24, 26, 35, 40, 41, and 45.

16. The polypeptide of any one of claims 6-15, wherein (Xaa)ₘ is represented by formula (IV)
(SEQ ID NO: 181)
aa1-aa2-aa3-aa4-aa5-aa6-aa7-aa8-aa9,
(IV)
wherein aa1 is an amino acid selected from Y, F, W, and N;
aa2 is an amino acid selected from K, P, H, A, and T;
aa3 is an amino acid selected from V, N, G, Q, A, and F;
aa4 is an amino acid selected from H, T, Y, W, K, V, and R;
aa5 is an amino acid selected from Q, S, G, P, and N;
aa6 is an amino acid selected from S, Y, E, L, K, and T;
aa7 is an amino acid selected from S, D, V, and K;
aa8 is an amino acid selected from G, L, S, P, H, D, and R; and
aa9 is an amino acid selected from G, Q, E, and A.

17. The polypeptide of any one of claims 6-16, wherein (Xaa)ₘ is an amino acid sequence having at least 80% or at least 90% identity to the amino acid sequence of any one of SEQ ID NOS: 57-108.

18. The polypeptide of claim 17, wherein (Xaa)ₘ is the amino acid sequence of any one of SEQ ID NOS: 57-108.

19. The polypeptide of any one of claims 6-16, wherein (Xaa)ₘ is an amino acid sequence having at least 80% or at least 90% identity to the amino acid sequence of any one of SEQ ID NOS: 75, 77, 79, 88, 93, 94, and 98.

20. The polypeptide of claim 19, wherein (Xaa)ₘ is an amino acid sequence of any one of SEQ ID NOS: 75, 77, 79, 88, 93, 94, and 98.

21. The polypeptide of any one of any one of claims 1-20, wherein the polypeptide comprises an amino acid sequence that has at least 70%, at least 80%, or at least 90% identity to an amino acid sequence of any one of SEQ ID NOS: 110-116 and 138.

22. The polypeptide of claim 21, wherein the polypeptide comprises an amino acid sequence of any one of SEQ ID NOS: 110-116 and 138.

23. The polypeptide of any one of claims 6-10, wherein (Xaa)ₙ is represented by formula (IV)
aa1-aa2-aa3-aa4-aa5-aa6-aa7-aa8-aa9 (IV),
wherein
aa1 is an amino acid with a neutral polar hydrophilic side chain;
aa2 is an amino acid with a neutral nonpolar hydrophobic side chain;
aa3 is an amino acid with a neutral nonpolar hydrophobic side chain;
aa4 is an amino acid with a neutral polar hydrophilic side chain;
aa5 is an amino acid with a positively charged polar hydrophilic side chain;
aa6 is an amino acid with a positively charged polar hydrophilic side chain;
aa7 is an amino acid with a neutral nonpolar hydrophobic side chain;
aa8 is an amino acid with a neutral nonpolar hydrophobic side chain; and
aa9 is an amino acid with a neutral nonpolar hydrophilic side chain.

24. The polypeptide of any one of claims 6-10, wherein (Xaa)ₘ is represented by formula (IV)
aa1-aa2-aa3-aa4-aa5-aa6-aa7-aa8-aa9 (IV)
wherein
aa1 is an amino acid with a neutral nonpolar hydrophobic side chain;
aa2 is an amino acid with a positively charged polar hydrophilic side chain;
aa3 is an amino acid with a neutral nonpolar hydrophobic side chain;
aa4 is an amino acid with a positively charged polar hydrophilic side chain;
aa5 is an amino acid with a neutral polar hydrophilic side chain;
aa6 is an amino acid with a neutral polar hydrophilic side chain;
aa7 is an amino acid with a negatively charged polar hydrophilic side chain;
aa8 is an amino acid with a positively charged polar hydrophilic side chain; and
aa9 is an amino acid with a neutral nonpolar hydrophilic side chain.

25. The polypeptide of claim 23 or 24, wherein
the amino acid with the neutral nonpolar hydrophilic side chain is selected from cysteine (C) and glycine (G);
the amino acid with the neutral nonpolar hydrophobic side chain is selected from alanine (A), isoleucine (I), leucine (L), methionine (M), phenylalanine (F), proline (P), tryptophan (W), and valine (V);
the amino acid with the neutral polar hydrophilic side chain is selected from asparagine (N), glutamine (Q), serine (S), threonine (T), and tyrosine (Y);
the amino acid with the positively charged polar hydrophilic side chain is selected from arginine (R), histidine (H), and lysine (K); and
the amino acid with the negatively charged polar hydrophilic side chain is selected from aspartate (D) and glutamate (E).

26. The polypeptide of any one of claims 1-25, wherein the polypeptide comprises an amino acid sequence that comprises a cysteine.

27. The polypeptide of claim 26, wherein the cysteine is available for chemical conjugation, and optionally wherein the cysteine is located at the C-terminal end or the N-terminal end of the polypeptide, and optionally wherein the polypeptide comprises the amino acid sequence of SEQ ID NO: 138.

28. The polypeptide of any one of claims 1-27 further comprising a heterologous polypeptide covalently linked through an amide bond to form a contiguous fusion protein.

29. The polypeptide of claim 28, wherein the heterologous polypeptide comprises a therapeutic polypeptide.

30. The polypeptide of claim 29, wherein the therapeutic polypeptide is selected from the group consisting of polypeptide hormones, polypeptide cytokines, polypeptide chemokines, growth factors, hemostasis active polypeptides, enzymes, and toxins.

31. The polypeptide of claim 29, wherein the therapeutic polypeptide is selected from the group consisting of receptor traps and receptor ligands.

32. The polypeptide of claim 29, wherein the therapeutic polypeptide sequence is selected from the group consisting of angiogenic agents and anti-angiogenic agents.

33. The polypeptide of claim 29, wherein the therapeutic polypeptide sequence is a neurotransmitter, and optionally wherein the neurotransmitter is Neuropeptide Y.

34. The polypeptide of claim 29, wherein the therapeutic polypeptide sequence is an erythropoiesis-stimulating agent, and optionally wherein the erythropoiesis-stimulating agent is erythropoietin or an erythropoietin mimetic.

35. The polypeptide of claim 29, wherein the therapeutic polypeptide is an incretin, and optionally wherein the incretin is selected from the group consisting of glucagon, gastric inhibitory peptide (GIP), glucagon-like peptide-1 (GLP-1), glucagon-like peptide-2 (GLP-2), peptide YY (PYY), and oxyntomodulin (OXM).

36. A pharmaceutical composition suitable for therapeutic use in a human patient, comprising a polypeptide of any of claim 1-35, and a pharmaceutically acceptable excipient.

37. A polynucleotide comprising a sequence encoding the polypeptide of any of claims 1-36.

38. The polynucleotide of claim 37, wherein the sequence encoding the polypeptide is operably linked to a transcriptional regulatory sequence.

39. The polynucleotide of claim 38, wherein the transcriptional regulatory sequence is selected from the group consisting of promoters and enhancers.

40. The polynucleotide of claim 38 or 39, further comprising an origin of replication, a minichromosome maintenance element (MME), and/or a nuclear localization element.

41. The polynucleotide of any of claims 38-40, further comprising a polyadenylation signal sequence operably linked and transcribed with the sequence encoding the polypeptide.

42. The polynucleotide of any of claims 38-41, wherein the sequence encoding the polypeptide comprises at least one intronic sequence.

43. The polynucleotide of any of claims 38-42, further comprising at least one ribosome binding site transcribed with the sequence encoding the polypeptide.

44. The polynucleotide of any of claims 38-43, wherein the polynucleotide is a deoxyribonucleic acid (DNA).

45. The polynucleotide of any of claims 38-43, wherein the polynucleotide is a ribonucleic acid (RNA).

46. A viral vector comprising the polynucleotide of any of claims 38-45.

47. A plasmid or minicircle comprising the polynucleotide of any of claims 38-46.

48. A fusion protein comprising a polypeptide of any one of the preceding claims.

49. The fusion protein of claim 48 further comprising a linker.

50. The fusion protein of claim 49, wherein the linker is a rigid linker.

51. The fusion protein of claim 50, wherein the rigid linker comprises the sequence of SEQ ID NO: 161.

52. The fusion protein of claim 49, wherein the linker is a flexible linker.

53. The fusion protein of claim 50, wherein the flexible linker comprises the sequence of SEQ ID NO: 165.

54. The fusion protein of any one of claims 48-53 comprising two polypeptides of any one of the preceding claims.

55. The fusion protein of any one of claims 48-54 further comprising a therapeutic molecule.

56. The fusion protein of claim 55, wherein the therapeutic molecule is a therapeutic polypeptide.

57. The fusion protein of claim 56, wherein the therapeutic polypeptide is selected from hormones, cytokines, chemokines, growth factors, hemostasis active polypeptides, enzymes, and toxins, or is selected from antagonists of hormones, cytokines, chemokines, growth factors, hemostasis active polypeptides, enzymes, and toxins.

58. The fusion protein of any one of claims 48-57, wherein the polypeptide comprises the amino acid sequence of SEQ ID NO: 110.

59. The fusion protein of any one of claims 48-57, wherein the polypeptide comprises the amino acid sequence of SEQ ID NO: 113.

60. The fusion protein of any one of claims 48-57, wherein the polypeptide comprises the amino acid sequence of SEQ ID NO: 116.
